SEQUENCE LISTING (25 sequences)

SEQ ID NO: 1 - protein, 543 aa, Homo sapiens
MSSFSYEPYYSTSYKRRYVETPRVHISSVRSGYSTARSAYSSYSAPVSSSLSVRRSYSSSSGSLMPSLENLDLSQVAAISNDLKSIRTQEKAQLQDLNDRFASFIERVHELEQQNKVLEAELLVLRQKHSEPSRFRALYEQEIRDLRLAAEDATNEKQALQGEREGLEETLRNLQARYEEEVLSREDAEGRLMEARKGADEAALARAELEKRIDSLMDEISFLKKVHEEEIAELQAQIQYAQISVEMDVTKPDLSAALKDIRAQYEKLAAKNMQNAEEWFKSRFTVLTESAAKNTDAVRAAKDEVSESRRLLKAKTLEIEACRGMNEALEKQLQELEDKQNADISAMQDTINKLENELRTTKSEMARYLKEYQDLLNVKMALDIEIAAYRKLLEGEETRLSFTSVGSITSGYSQSSQVFGRSAYGGLQTSSYLMSTRSFPSYYTSHVQEEQIEVEETIEAAKAEEAKDEPPSEGEAEEEEKDKEEAEEEEAAEEEEAAKEESEEAKEEEEGGEGEEGEETKEAEEEEKKVEGAGEEQAAKKKD

SEQ ID NO: 2 - protein, 6 aa, Artificial Sequence
  Synthesized
YVETPR

SEQ ID NO: 3 - protein, 7 aa, Artificial Sequence
  Synthesized
VHISSVR

SEQ ID NO: 4 - protein, 17 aa, Artificial Sequence
  Synthesized
SAYSSYSAPVSSSLSVR

SEQ ID NO: 5 - protein, 29 aa, Artificial Sequence
  Synthesized
SYSSSSGSLMPSLENLDLSQVAAISNDLK

SEQ ID NO: 6 - protein, 9 aa, Artificial Sequence
  Synthesized
AQLQDLNDR

SEQ ID NO: 7 - protein, 7 aa, Artificial Sequence
  Synthesized
FASFIER

SEQ ID NO: 8 - protein, 9 aa, Artificial Sequence
  Synthesized
VHELEQQNK

SEQ ID NO: 9 - protein, 10 aa, Artificial Sequence
  Synthesized
VLEAELLVLR

SEQ ID NO: 10 - protein, 8 aa, Artificial Sequence
  Synthesized
ALYEQEIR

SEQ ID NO: 11 - protein, 10 aa, Artificial Sequence
  Synthesized
LAAEDATNEK

SEQ ID NO: 12 - protein, 7 aa, Artificial Sequence
  Synthesized
QALQGER

SEQ ID NO: 13 - protein, 8 aa, Artificial Sequence
  Synthesized
EGLEETLR

SEQ ID NO: 14 - protein, 8 aa, Artificial Sequence
  Synthesized
YEEEVLSR

SEQ ID NO: 15 - protein, 5 aa, Artificial Sequence
  Synthesized
LMEAR

SEQ ID NO: 16 - protein, 9 aa, Artificial Sequence
  Synthesized
GADEAALAR

SEQ ID NO: 17 - protein, 12 aa, Artificial Sequence
  Synthesized
IDSLMDEISFLK

SEQ ID NO: 18 - protein, 10 aa, Artificial Sequence
  Synthesized
FTVLTESAAK

SEQ ID NO: 19 - protein, 8 aa, Artificial Sequence
  Synthesized
GMNEALEK

SEQ ID NO: 20 - protein, 8 aa, Artificial Sequence
  Synthesized
QLQELEDK

SEQ ID NO: 21 - protein, 6 aa, Artificial Sequence
  Synthesized
LENELR

SEQ ID NO: 22 - protein, 22 aa, Artificial Sequence
  Synthesized
LSFTSVGSITSGYSQSSQVFGR

SEQ ID NO: 23 - protein, 16 aa, Artificial Sequence
  Synthesized
SAYGGLQTSSYLMSTR

SEQ ID NO: 24 - protein, 25 aa, Artificial Sequence
  Synthesized
SFPSYYTSHVQEEQIEVEETIEAAK

SEQ ID NO: 25 - protein, 11 aa, Artificial Sequence
  Synthesized
VEGAGEEQAAK

(19) United States
(12) Patent Application Publication
(10) Pub. No.: US 2025/0020669 A1
(43) Pub. Date: Jan. 16, 2025

(54) METHODS FOR DETECTING NEUROFILAMENT LIGHT CHAIN IN PLASMA AND CEREBROSPINAL FLUID

(71) Applicant: Washington University, St. Louis, MO (US)

(72) Inventors: Randall Bateman, St. Louis, MO (US); Melissa Budelier, St. Louis, MO (US); David Holtzman, St. Louis, MO (US); Hong Jiang, St. Louis, MO (US)

(21) Appl. No.: 18/276,710

(22) PCT Filed: Feb. 10, 2022
(86) PCT No.: PCT/US22/15998

§ 371 Date: Aug. 10, 2023

Related U.S. Application Data
(60) Provisional application No. 63/147,833, filed on Feb. 10, 2021.
(60) Provisional application No. 63/183,417, filed on May 3, 2021.
(60) Provisional application No. 63/197,826, filed on Jun. 7, 2021.

(51) Int. Cl. G01N 33/68; C07K 16/18

(52) U.S. Cl. CPC G01N 33/6896; C07K 16/18; C07K 2317/34; G01N 2800/28; G01N 2800/52

(57) ABSTRACT

The present disclosure provides methods to detect and optionally quantify Nfl present in cerebrospinal fluid and blood and use of the methods to detect and optionally measure levels of Nfl biomarkers indicative of neuronal damage. Also disclosed are anti-Nfl antibodies.

### CROSS REFERENCE TO RELATED APPLICATIONS

This application claims priority of U.S. Provisional application No. 63/147,833 filed Feb. 10, 2021, U.S. Provisional application No. 63/183,417, filed May 3, 2021, and U.S. Provisional application No. 63/197,826, filed Jun. 7, 2021, each of which is hereby incorporated by reference in its entirety.

### GOVERNMENTAL RIGHTS

This invention was made with government support under AG067559 awarded by the National Institutes of Health. The government has certain rights in the invention.

### REFERENCE TO A SEQUENCE LISTING

This application contains a Sequence Listing that has been submitted in ASCII format via EFS-Web and is hereby incorporated by reference in its entirety. The ASCII copy, created on Jan. 31, 2022, is named “716468_ST25.txt”, and is 10,025 bytes in size.

### FIELD OF THE TECHNOLOGY

The present disclosure encompasses methods to quantify and analyze various neurofilament light chain species and the uses thereof to measure neuronal damage, inform diagnosis of neurodegenerative diseases, select patients for further diagnostic testing, and guide treatment decisions.

### BACKGROUND

Neurofilaments are a major component of the neuronal cytoskeleton of myelinated axons and play an important role in nerve conductance by allowing radial axonal growth. In the central nervous system, neurofilaments are composed of four proteins: Neurofilament heavy chain (NfH), neurofilament medium chain (NfM), neurofilament light chain (NfL) and alpha-internexin. Among these four proteins, only NfL is well established as a marker of neuronal damage. It is elevated in Alzheimer's disease (AD), frontotemporal dementia (FTD), Parkinson's disease (PD), progressive supranuclear palsy (PSP), traumatic brain injury (TBI), multiple sclerosis (MS), amyotrophic lateral sclerosis (ALS), and other neurodegenerative disorders. This indicates that the biology and pathophysiology learned about NfL is useful for a variety of neurodegenerative diseases.

Currently, only one commercially available pair of anti-Nfl antibodies are utilized for monitoring clinical cohorts and trials (anti-NF-L mAb 2:1 and anti-Nfl mAb 47:3, Umam). While use of the commercial assay has been successful at differentiating neurodegenerative diseases from healthy controls, the biomarker's utility is currently limited by its nonspecific nature. “Noise” due to Nfl elevations in multiple neurodegenerative and neuroinflammatory processes make Nfl a less reliable marker of disease status and treatment response, limiting its clinical utility in treatment protocols. While Nfl undergoes multiple post-translational modifications (PTMs), resulting in the possibility of many different Nfl isoforms in any given biological sample, the commercially available immunoassay is blind to these PTMs. Moreover, important gaps remain in our understanding, for example, to what extent, if any, Nfl species can be used to detect neurodegenerative disorders, stage subjects, and/or guide treatment decisions.

Accordingly, there remains a need in the art for improved methods to detect and quantify Nfl isoforms present in cerebrospinal fluid and blood.

### BRIEF DESCRIPTION OF THE FIGURES

The application file contains at least one photograph executed in color. Copies of this patent application publication with color photographs will be provided by the Office upon request and payment of the necessary fee.

FIG. 1 is a graph showing amounts of recombinant, full-length Nfl (rec-Nfl) after immunoprecipitation with an anti-Nfl antibody and LC-MS analysis. The y-axis is 14N/15N ratio of the measured peak area for each tryptic peptide along the x-axis. See the first paragraph of Example 2 for a description of the tryptic peptide nomenclature. Each line is a different antibody (see the key within the figure). Data for individual antibodies are presented in FIG. 4-27.

FIG. 2A is a graph showing amounts of Nfl immunoprecipitated from brain lysate with an anti-Nfl antibody as measured by LC-MS. The y-axis is 14N/15N ratio of the measured peak area for each tryptic peptide identified along the x-axis. See the first paragraph of Example 2 for a description of the tryptic peptide nomenclature. Each line is a different antibody (see the key within the figure). Data for individual antibodies are presented in FIG. 4-21.

FIG. 2B is an enlarged version of the graph shown FIG. 2A (note the difference in the scale of the y-axis between the two figures).

FIG. 3 is a graph showing amounts of Nfl immunoprecipitated from CSF with an anti-Nfl antibody as measured by LC-MS. The y-axis is 14N/15N ratio of the measured peak area for each tryptic peptide identified along the x-axis. See the first paragraph of Example 2 for a description of the tryptic peptide nomenclature. Each line is a different antibody (see the key within the figure). Data for individual antibodies are presented in FIG. 4-27.

FIG. 4A and FIG. 4B are graphs showing amounts of Nfl immunoprecipitated from brain lysate (FIG. 4A) and CSF (FIG. 4B) with an anti-Nfl antibody, HJ30.1, as measured by LC-MS. The y-axis is 14N/15N ratio of the measured peak area for each tryptic peptide identified along the x-axis. See the first paragraph of Example 2 for a description of the tryptic peptide nomenclature.

FIG. 4C is a graph showing amounts rec-Nfl after immunoprecipitation with an anti-Nfl antibody, HJ30.1, as measured by LC-MS. The y-axis is 14N/15N ratio of the measured peak area for each tryptic peptide identified along the x-axis. See the first paragraph of Example 2 for a description of the tryptic peptide nomenclature.

FIG. 5A and FIG. 5B are graphs showing amounts of Nfl immunoprecipitated from brain lysate (FIG. 5A) and CSF (FIG. 5B) with an anti-Nfl antibody, HJ30.2, as measured by LC-MS. The y-axis is 14N/15N ratio of the measured peak area for each tryptic peptide identified along the x-axis. See the first paragraph of Example 2 for a description of the tryptic peptide nomenclature.

FIG. 5C is a graph showing amounts of rec-Nfl after immunoprecipitation with an anti-Nfl antibody, HJ30.2, as measured by LC-MS. The y-axis is 14N/15N ratio of the measured peak area for each tryptic peptide identified along the x-axis. See the first paragraph of Example 2 for a description of the tryptic peptide nomenclature.

FIG. 6A and FIG. 6B are graphs showing amounts of Nfl immunoprecipitated from brain lysate (FIG. 6A) and CSF (FIG. 6B) with an anti-Nfl antibody, HJ30.3.1, as measured by LC-MS. The y-axis is 14N/15N ratio of the measured peak area for each tryptic peptide identified along the x-axis. See the first paragraph of Example 2 for a description of the tryptic peptide nomenclature.

FIG. 6C is a graph showing amounts of tryptic peptides of rec-Nfl after immunoprecipitation with an anti-Nfl antibody, HJ30.3.1, as measured by LC-MS. The y-axis is 14N/15N ratio of the measured peak area for each tryptic peptide identified along the x-axis. See the first paragraph of Example 2 for a description of the tryptic peptide nomenclature.

FIG. 7A and FIG. 7B are graphs showing amounts of Nfl immunoprecipitated from brain lysate (FIG. 7A) and CSF (FIG. 7B) with an anti-Nfl antibody, HJ30.4, as measured by LC-MS. The y-axis is 14N/15N ratio of the measured peak area for each tryptic peptide identified along the x-axis. See the first paragraph of Example 2 for a description of the tryptic peptide nomenclature.

FIG. 7C is a graph showing amounts of rec-Nfl after immunoprecipitation with an anti-Nfl antibody, HJ30.4, as measured by LC-MS. The y-axis is 14N/15N ratio of the measured peak area for each tryptic peptide identified along the x-axis. See the first paragraph of Example 2 for a description of the tryptic peptide nomenclature.

FIG. 8A and FIG. 8B are graphs showing amounts of Nfl immunoprecipitated from brain lysate (FIG. 8A) and CSF (FIG. 8B) with an anti-Nfl antibody, HJ30.3.5, as measured by LC-MS. The y-axis is 14N/15N ratio of the measured peak area for each tryptic peptide identified along the x-axis. See the first paragraph of Example 2 for a description of the tryptic peptide nomenclature.

FIG. 8C is a graph showing amounts of rec-Nfl after immunoprecipitation with an anti-Nfl antibody, HJ30.5, as measured by LC-MS. The y-axis is 14N/15N ratio of the measured peak area for each tryptic peptide along the x-axis. See the first paragraph of Example 2 for a description of the tryptic peptide nomenclature.

FIG. 9A and FIG. 9B are graphs showing amounts of of Nfl immunoprecipitated from brain lysate immunoprecipitated from brain lysate (FIG. 9A) and CSF (FIG. 9B) with an anti-Nfl antibody, HJ30.6, as measured by LC-MS. The y-axis is 14N/15N ratio of the measured peak area for each tryptic peptide identified along the x-axis. See the first paragraph of Example 2 for a description of the tryptic peptide nomenclature.

FIG. 9C is a graph showing amounts of rec-Nfl after immunoprecipitation with an anti-Nfl antibody, HJ30.6, as measured by LC-MS. The y-axis is 14N/15N ratio of the measured peak area for each tryptic peptide identified along the x-axis. See the first paragraph of Example 2 for a description of the tryptic peptide nomenclature.

FIG. 10A and FIG. 10B are graphs showing amounts of Nfl immunoprecipitated from brain lysate (FIG. 10A) and CSF (FIG. 10B) with an anti-Nfl antibody, HJ30.7, as measured by LC-MS. The y-axis is 14N/15N ratio of the measured peak area for each tryptic peptide identified along the x-axis. See the first paragraph of Example 2 for a description of the tryptic peptide nomenclature.

FIG. 10C is a graph showing amounts of rec-Nfl after immunoprecipitation with an anti-Nfl antibody, HJ30.7, as measured by LC-MS. The y-axis is 14N/15N ratio of the measured peak area for each tryptic peptide identified along the x-axis. See the first paragraph of Example 2 for a description of the tryptic peptide nomenclature.

FIG. 11A and FIG. 11B are graphs showing amounts of Nfl immunoprecipitated from brain lysate (FIG. 11A) and CSF (FIG. 11B) with an anti-Nfl antibody, HJ30.8, as measured by LC-MS. The y-axis is 14N/15N ratio of the measured peak area for each tryptic peptide identified along the x-axis. See the first paragraph of Example 2 for a description of the tryptic peptide nomenclature.

FIG. 11C is a graph showing amounts of rec-Nfl after immunoprecipitation with an anti-Nfl antibody, HJ30.8, as measured by LC-MS. The y-axis is 14N/15N ratio of the measured peak area for each tryptic peptide identified along the x-axis. See the first paragraph of Example 2 for a description of the tryptic peptide nomenclature.

FIG. 12A and FIG. 12B are graphs showing amounts of Nfl immunoprecipitated from brain lysate (FIG. 12A) and CSF (FIG. 12B) with an anti-Nfl antibody, HJ30.9, as measured by LC-MS. The y-axis is 14N/15N ratio of the measured peak area for each tryptic peptide identified along the x-axis. See the first paragraph of Example 2 for a description of the tryptic peptide nomenclature.

FIG. 12C is a graph showing amounts of rec-Nfl after immunoprecipitation with an anti-Nfl antibody, HJ30.9, as measured by LC-MS. The y-axis is 14N/15N ratio of the measured peak area for each tryptic peptide identified along the x-axis. See the first paragraph of Example 2 for a description of the tryptic peptide nomenclature.

FIG. 13A and FIG. 13B are graphs showing amounts of Nfl immunoprecipitated from brain lysate (FIG. 13A) and CSF (FIG. 13B) with an anti-Nfl antibody, HJ30.11, as measured by LC-MS. The y-axis is 14N/15N ratio of the measured peak area for each tryptic peptide identified along the x-axis. See the first paragraph of Example 2 for a description of the tryptic peptide nomenclature.

FIG. 13C is a graph showing amounts of rec-Nfl after immunoprecipitation with an anti-Nfl antibody, HJ30.11, as measured by LC-MS. The y-axis is 14N/15N ratio of the measured peak area for each tryptic peptide identified along the x-axis. See the first paragraph of Example 2 for a description of the tryptic peptide nomenclature.

FIG. 14A and FIG. 14B are graphs showing amounts of Nfl immunoprecipitated from brain lysate (FIG. 14A) and CSF (FIG. 14B) with an anti-Nfl antibody, HJ30.12, as measured by LC-MS. The y-axis is 14N/15N ratio of the measured peak area for each tryptic peptide identified along the x-axis. See the first paragraph of Example 2 for a description of the tryptic peptide nomenclature.

FIG. 14C is a graph showing amounts of rec-Nfl after immunoprecipitation with an anti-Nfl antibody, HJ30.12, as measured by LC-MS. The y-axis is 14N/15N ratio of the measured peak area for each tryptic peptide identified along the x-axis. See the first paragraph of Example 2 for a description of the tryptic peptide nomenclature.

FIG. 15A and FIG. 15B are graphs showing amounts of Nfl immunoprecipitated from brain lysate (FIG. 15A) and CSF (FIG. 15B) with an anti-Nfl antibody, HJ30.13, as measured by LC-MS. The y-axis is 14N/15N ratio of the measured peak area for each tryptic peptide identified along the x-axis. See the first paragraph of Example 2 for a description of the tryptic peptide nomenclature.

FIG. 15C is a graph showing amounts of rec-Nfl after immunoprecipitation with an anti-Nfl antibody, HJ30.13, as measured by LC-MS. The y-axis is 14N/15N ratio of the measured peak area for each tryptic peptide identified along the x-axis. See the first paragraph of Example 2 for a description of the tryptic peptide nomenclature.

FIG. 16A and FIG. 16B are graphs showing amounts of Nfl immunoprecipitated from brain lysate (FIG. 16A) and CSF (FIG. 16B) with an anti-Nfl antibody, HJ30.14, as measured by LC-MS. See the first paragraph of Example 2 for a description of the tryptic peptide nomenclature.

FIG. 16C is a graph showing amounts of rec-Nfl after immunoprecipitation with an anti-Nfl antibody, HJ30.14, as measured by LC-MS. The y-axis is 14N/15N ratio of the measured peak area for each tryptic peptide identified along the x-axis. See the first paragraph of Example 2 for a description of the tryptic peptide nomenclature.

FIG. 17A and FIG. 17B are graphs showing amounts of Nfl immunoprecipitated from brain lysate (FIG. 17A) and CSF (FIG. 17B) with an anti-Nfl antibody, HJ30.15, as measured by LC-MS. The y-axis is 14N/15N ratio of the measured peak area for each tryptic peptide identified along the x-axis. See the first paragraph of Example 2 for a description of the tryptic peptide nomenclature.

FIG. 17C is a graph showing amounts rec-Nfl after immunoprecipitation with an anti-Nfl antibody, HJ30.15, as measured by LC-MS. The y-axis is 14N/15N ratio of the measured peak area for each tryptic peptide identified along the x-axis. See the first paragraph of Example 2 for a description of the tryptic peptide nomenclature.

FIG. 18A and FIG. 18B are graphs showing amounts of Nfl immunoprecipitated from brain lysate (FIG. 18A) and CSF (FIG. 18B) with an anti-Nfl antibody, HJ30.16.1, as measured by LC-MS. The y-axis is 14N/15N ratio of the measured peak area for each tryptic peptide identified along the x-axis. See the first paragraph of Example 2 for a description of the tryptic peptide nomenclature.

FIG. 18C is a graph showing amounts of rec-Nfl after immunoprecipitation with an anti-Nfl antibody, HJ30.16.1, as measured by LC-MS. The y-axis is 14N/15N ratio of the measured peak area for each tryptic peptide identified along the x-axis. See the first paragraph of Example 2 for a description of the tryptic peptide nomenclature.

FIG. 19A and FIG. 19B are graphs showing amounts of Nfl immunoprecipitated from brain lysate (FIG. 19A) and CSF (FIG. 19B) with an anti-Nfl antibody, HJ30.16.2, as measured by LC-MS. The y-axis is 14N/15N ratio of the measured peak area for each tryptic peptide identified along the x-axis. See the first paragraph of Example 2 for a description of the tryptic peptide nomenclature.

FIG. 19C is a graph showing amounts of rec-Nfl after immunoprecipitation with an anti-Nfl antibody, HJ30.16.2, as measured by LC-MS. The y-axis is 14N/15N ratio of the measured peak area for each tryptic peptide identified along the x-axis. See the first paragraph of Example 2 for a description of the tryptic peptide nomenclature.

FIG. 20A and FIG. 20B are graphs showing amounts of Nfl immunoprecipitated from brain lysate (FIG. 20A) and CSF (FIG. 20B) with an anti-Nfl antibody, HJ30.17, as measured by LC-MS. The y-axis is 14N/15N ratio of the measured peak area for each tryptic peptide identified along the x-axis. See the first paragraph of Example 2 for a description of the tryptic peptide nomenclature.

FIG. 20C is a graph showing amounts of rec-Nfl after immunoprecipitation with an anti-Nfl antibody, HJ30.17, as measured by LC-MS. The y-axis is 14N/15N ratio of the measured peak area for each tryptic peptide identified along the x-axis. See the first paragraph of Example 2 for a description of the tryptic peptide nomenclature.

FIG. 21A and FIG. 21B are graphs showing amounts of Nfl immunoprecipitated from brain lysate (FIG. 21A) and CSF (FIG. 21B) with an anti-Nfl antibody, HJ30.18, as measured by LC-MS. The y-axis is 14N/15N ratio of the measured peak area for each tryptic peptide identified along the x-axis. See the first paragraph of Example 2 for a description of the tryptic peptide nomenclature.

FIG. 21C is a graph showing amounts of rec-Nfl after immunoprecipitation with an anti-Nfl antibody, HJ30.18, as measured by LC-MS. The y-axis is 14N/15N ratio of the measured peak area for each tryptic peptide identified along the x-axis. See the first paragraph of Example 2 for a description of the tryptic peptide nomenclature.

FIG. 22 is an illustration summarizing data from the anti-Nfl antibody immunoprecipitation experiments and MS analysis (individual data shown in FIGS. 4-21). The top illustration shows approximate Nfl isoforms enriched from CSF. The bottom illustration shows approximate Nfl isoforms enriched from brain lysate. Each illustration contains a schematic of full-length Nfl protein with amino acid numbering indicated along the bottom. In CSF, a plurality of Nfl isoforms were enriched including isoforms with N-terminal truncations, isoforms with C-terminal truncations, and isoforms with N-terminal and C-terminal truncations. Isoform size is approximated from the MS data, but is not specific at the residue level due to technical limitations of the approach used. Therefore, each line may represent a plurality of isoforms with differences in length at either terminus. A solid line (dark blue) indicates a higher level of confidence—the truncated isoform contains residues approximated by the solid line. The dashed line (light blue) indicates a lower level of confidence, representing a possible variation. For instance, the MS data suggests a plurality of N-terminally truncated isoforms that contain amino acids 530 to 540 of SEQ ID NO: 1 (represented by the four bottom lines in the CSF illustration). The smallest of these isoforms is approximated to contain amino acids 462 to 543 of SEQ ID NO: 1. This is only an approximation however; the exact length cannot be determined using the current approach. It is also possible therefore the smallest isoform represented is a plurality of isoforms. In brain lysate, at least two populations of Nfl isoforms were enriched—a first population that is approximately full-length and a second population of N-terminally truncated isoforms that comprise at least amino acids 530 to 540 of SEQ ID NO: 1. The CSF illustration also includes an approximate indication of various regions that contain epitopes to which anti-Nfl antibodies bind (indicated above the Nfl protein schematic). For instance, anti-Nfl antibodies HJ30.1, HJ30.2, HJ30.13 and HJ30.15 are depicted as binding to epitopes within a region comprising about amino acid 100 to about 225 of full-length Nfl, as determined from the MS data. Note: this representation is not suggesting that these antibodies bind the same epitope.

FIG. 23 depicts a schematic of a two-step enrichment process (left side) and a graph showing amounts (y-axis, peak area) of the tryptic peptide [323,330] immunoprecipitated with HJ30.4 in each step. Red bars are 14N [323,330] and blue bars are the 15N [323,330]. See the first paragraph of Example 2 for a description of the tryptic peptide nomenclature.

FIG. 24 depicts a schematic of a two-step enrichment process (left side) and a graph showing amounts (y-axis, peak area) of the tryptic peptide [529,539] immunoprecipitated with HJ30.11 in each step. Red bars are 14N [529,539] and blue bars are the 15N [529,539]. See the first paragraph of Example 2 for a description of the tryptic peptide nomenclature.

FIG. 25 depicts a schematic of a two-step enrichment process (left side) and a graph showing amounts (y-axis, peak area) of the tryptic peptide [116, 125] immunoprecipitated with HJ30.13 in each step. Red bars are 14N [116, 125] and blue bars are the 15N [116, 125]. See the first paragraph of Example 2 for a description of the tryptic peptide nomenclature.

FIG. 26 depicts two different three-step enrichment processes. The sample names identified correspond to the keys for FIG. 27-28.

FIG. 27 is a graph showing amounts of Nfl as measured by LC-MS. The y-axis is 14N/15N ratio of the measured peak area for each tryptic peptide identified along the x-axis. See the first paragraph of Example 2 for a description of the tryptic peptide nomenclature. Each colored line is a different sample, as identified in the key and FIG. 26.

FIG. 28 is a graph showing amounts of Nfl as measured by LC-MS. The y-axis is 14N peak area for each tryptic identified peptide along the x-axis. See the first paragraph of Example 2 for a description of the tryptic peptide nomenclature. Each colored line is a different sample, as identified in the key and FIG. 26.

FIG. 29 depicts a three-step enrichment process. The sample names identified correspond to FIGS. 30-39 and 41.

FIG. 30 is a graph showing amounts of Nfl enriched from CSF obtained from a subject that was amyloid positive with a CDR=0.5. The y-axis is 14N/15N ratio of the measured peak area for each tryptic peptide along the x-axis. The identity of the tryptic peptides, which are numbered 1 to 24 along the x-axis, is provided in Table A. The blue line is the “30.13” sample, the orange line is the “post 30.13_30.4|P” sample; and the grey line is the “post 30.13&4_30.11IP” sample.

TABLE A

| Tryptic | | m/z and | No. on the |
|---|---|---|---|
| peptide | Sequence (SEQ ID NO) | charge | x-axis |
| [17, 22] | YVETPR (SEQ ID NO: 2) | 382.7005++ | 1 |
| [23, 29] | VHISSVR (SEQ ID NO: 3) | 399.2350++ | 2 |
| [37, 53] | SAYSSYSAPVSSSLSVR (SEQ ID NO: 4) | 874.4285++ | 3 |
| [55, 83] | SYSSSSGSLMPSLENLDLSQVAAISNDLK | 1005.1605+++ | 4 |
| | (SEQ ID NO: 5) | | |
| [91, 99] | AQLQDLNDR (SEQ ID NO: 6) | 536.7727++ | 5 |
| [100, 106] | FASFIER (SEQ ID NO: 7) | 435.2294++ | 6 |
| [107, 115] | VHELEQQNK (SEQ ID NO: 8) | 562.7884++ | 7 |
| [116, 125] | VLEAELLVLR (SEQ ID NO: 9) | 577.8608++ | 8 |
| [136, 143] | ALYEQEIR (SEQ ID NO: 10) | 511.2693++ | 9 |
| [147, 156] | LAAEDATNEK (SEQ ID NO: 11) | 531.2591++ | 10 |
| [157, 163] | QALQGER (SEQ ID NO: 12) | 401.2143++ | 11 |
| [164, 171] | EGLEETLR (SEQ ID NO: 13) | 473.7456++ | 12 |
| [177, 184] | YEEEVLSR (SEQ ID NO: 14) | 512.7509++ | 13 |
| [191, 195] | LMEAR (SEQ ID NO: 15) | 310.1652++ | 14 |
| [197, 205] | GADEAALAR (SEQ ID NO: 16) | 437.2249++ | 15 |
| [212, 223] | IDSLMDEISFLK (SEQ ID NO: 17) | 705.8629++ | 16 |
| [283, 292] | FTVLTESAAK (SEQ ID NO: 19) | 533.7926++ | 17 |
| [323, 330] | GMNEALEK (SEQ ID NO: 20) | 446.2157++ | 18 |
| [331, 338] | QLQELEDK (SEQ ID NO: 21) | 501.7587++ | 19 |
| [353, 358] | LENELR (SEQ ID NO: 22) | 387.2112++ | 20 |
| [399, 420] | LSFTSVGSITSGYSQSSQVFGR (SEQ ID NO: 26) | 765.7132+++ | 21 |
| [421, 436] | SAYGGLQTSSYLMSTR (SEQ ID NO: 27) | 861.4118++ | 22 |
| [437, 461] | SFPSYYTSHVQEEQIEVEETIEAAK (SEQ ID NO: 28) | 972.1258+++ | 23 |
| [529, 539] | VEGAGEEQAAK (SEQ ID NO: 29) | 544.7646++ | 24 |

FIG. 31 is a graph showing amounts of Nfl enriched from CSF obtained from a subject that was amyloid positive with a CDR=0.5. The y-axis is 14N/15N ratio of the measured peak area for each tryptic peptide along the x-axis. The identity of the tryptic peptides, which are numbered 1 to 24 along the x-axis, is provided in Table A. The blue line is the “30.13” sample, the orange line is the “post 30.13_30.4IP” sample; and the grey line is the “post 30.13&4_30.11IP” sample.

FIG. 32 is a graph showing amounts of Nfl enriched from CSF obtained from a subject that was amyloid positive with a CDR=0.5. The y-axis is 14N/15N ratio of the measured peak area for each tryptic peptide along the x-axis. The identity of the tryptic peptides, which are numbered 1 to 24 along the x-axis, is provided in Table A. The blue line is the “30.13” sample, the orange line is the “post 30.13_30.4IP” sample; and the grey line is the “post 30.13&4_30.11IP” sample.

FIG. 33 is a graph showing amounts of Nfl enriched from CSF obtained from a subject that was amyloid positive with a CDR=0.5. The y-axis is 14N/15N ratio of the measured peak area for each tryptic peptide along the x-axis. The identity of the tryptic peptides, which are numbered 1 to 24 along the x-axis, is provided in Table A. The blue line is the “30.13” sample, the orange line is the “post 30.13_30.4IP” sample; and the grey line is the “post 30.13&4_30.11IP” sample.

FIG. 34 is a graph showing amounts of Nfl enriched from CSF obtained from a subject that was amyloid negative with a CDR=0. The y-axis is 14N/15N ratio of the measured peak area for each tryptic peptide along the x-axis. The identity of the tryptic peptides, which are numbered 1 to 24 along the x-axis, is provided in Table A. The blue line is the “30.13” sample, the orange line is the “post 30.13_30.4IP” sample; and the grey line is the “post 30.13&4_30.11IP” sample.

FIG. 35 is a graph showing amounts of Nfl enriched from CSF obtained from a subject that was amyloid negative with a CDR=0. The y-axis is 14N/15N ratio of the measured peak area for each tryptic peptide along the x-axis. The identity of the tryptic peptides, which are numbered 1 to 24 along the x-axis, is provided in Table A. The blue line is the “30.13” sample, the orange line is the “post 30.13_30.4IP” sample; and the grey line is the “post 30.13&4_30.11IP” sample.

FIG. 36 is a graph showing amounts of Nfl enriched from CSF obtained from a subject that was amyloid negative with a CDR=0. The y-axis is 14N/15N ratio of the measured peak area for each tryptic peptide along the x-axis. The identity of the tryptic peptides, which are numbered 1 to 24 along the x-axis, is provided in Table A. The blue line is the “30.13” sample, the orange line is the “post 30.13_30.4IP” sample; and the grey line is the “post 30.13&4_30.11IP” sample.

FIG. 37 is a graph showing amounts of Nfl enriched from CSF obtained from a subject that was amyloid negative with a CDR=0. The y-axis is 14N/15N ratio of the measured peak area for each tryptic peptide along the x-axis. The identity of the tryptic peptides, which are numbered 1 to 24 along the x-axis, is provided in Table A. The blue line is the “30.13” sample, the orange line is the “post 30.13_30.4IP” sample; and the grey line is the “post 30.13&4_30.11IP” sample.

FIG. 38 is a graph showing amounts of Nfl enriched from CSF obtained from a subject that was amyloid negative with a CDR=0. The y-axis is 14N/15N ratio of the measured peak area for each tryptic peptide along the x-axis. The identity of the tryptic peptides, which are numbered 1 to 24 along the x-axis, is provided in Table A. The blue line is the “30.13” sample, the orange line is the “post 30.13_30.4IP” sample; and the grey line is the “post 30.13&4_30.11IP” sample.

FIG. 39 is a graph showing amounts of Nfl enriched from CSF obtained from a subject that was amyloid negative with a CDR=0. The y-axis is 14N/15N ratio of the measured peak area for each tryptic peptide along the x-axis. The identity of the tryptic peptides, which are numbered 1 to 24 along the x-axis, is provided in Table A. The blue line is the “30.13” sample, the orange line is the “post 30.13_30.4|P” sample; and the grey line is the “post 30.13&4_30.11IP” sample.

FIG. 40A, FIG. 40B, and FIG. 40C are graphs summarizing the individual data in FIGS. 31-40. The y-axis is 14N/15N ratio of the measured peak area for each tryptic peptide identified along the x-axis. The red lines are the amyloid positive subjects and the black lines are the amyloid negative subjects.

FIG. 40D is a graph summarizing the individual data in FIGS. 31-40. The x-axis is 14N/15N ratio of the measured peak area for each tryptic peptide along the y-axis.

FIG. 41A and FIG. 41B are graphs showing amounts of Nfl enriched from CSF obtained from subjects that were amyloid positive (“AD”) and CDR 0, CDR 0.5, CDR 1, or CDR 2, or from subjects that were amyloid negative and CDR 0, CDR 0.5, or CDR 1. Subjects with CDR≥0.5 that are amyloid negative are considered “non-AD.” FIG. 41A shows the “post30.13_30.4IP sample”, and FIG. 41B shows the “post 30.13&4_30.11IP sample”. In each graph, the y-axis is 14N/15N ratio of the measured peak area for each tryptic peptide along the x-axis. The identity of the tryptic peptides, which are numbered 1 to 24 along the x-axis, is provided in Table B.

TABLE B

| Tryptic | | m/z and | No. on the |
|---|---|---|---|
| peptide | Sequence (SEQ ID NO) | charge | x-axis |
| [17, 22] | YVETPR (SEQ ID NO: 2) | 382.7005++ | 1 |
| [23, 29] | VHISSVR (SEQ ID NO: 3) | 399.2350++ | 2 |
| [37, 53] | SAYSSYSAPVSSSLSVR (SEQ ID NO: 4) | 874.4285++ | 3 |
| [55, 83] | SYSSSSGSLMPSLENLDLSQVAAISNDLK | 1005.1605+++ | 4 |
| | (SEQ ID NO: 5) | | |
| [91, 99] | AQLQDLNDR (SEQ ID NO: 6) | 536.7727++ | 5 |
| [100, 106] | FASFIER (SEQ ID NO: 7) | 435.2294++ | 6 |
| [107, 115] | VHELEQQNK (SEQ ID NO: 8) | 562.7884++ | 7 |
| [116, 125] | VLEAELLVLR (SEQ ID NO: 9) | 577.8608++ | 8 |
| [136, 143] | ALYEQEIR (SEQ ID NO: 10) | 511.2693++ | 9 |
| [147, 156] | LAAEDATNEK (SEQ ID NO: 11) | 531.2591++ | 10 |
| [157, 163] | QALQGER (SEQ ID NO: 12) | 401.2143++ | 11 |
| [164, 171] | EGLEETLR (SEQ ID NO: 13) | 473.7456++ | 12 |
| [177, 184] | YEEEVLSR (SEQ ID NO: 14) | 512.7509++ | 13 |
| [191, 195] | LMEAR (SEQ ID NO: 15) | 310.1652++ | 14 |
| [197, 205] | GADEAALAR (SEQ ID NO: 16) | 437.2249++ | 15 |
| [212, 223] | IDSLMDEISFLK (SEQ ID NO: 17) | 705.8629++ | 16 |
| [271, 280] | NMQNAEEWFK (SEQ ID NO: 18) | 648.7875++ | 17 |
| [283, 292] | FTVLTESAAK (SEQ ID NO: 19) | 533.7926++ | 18 |
| [323, 330] | GMNEALEK (SEQ ID NO: 20) | 446.2157++ | 19 |
| [331, 338] | QLQELEDK (SEQ ID NO: 21) | 501.7587++ | 20 |
| [353, 358] | LENELR (SEQ ID NO: 22) | 387.2112++ | 21 |
| [370, 378] | EYQDLLNVK (SEQ ID NO: 23) | 561.2955++ | 22 |
| [379, 389] | MALDIEIAAYR (SEQ ID NO: 24) | 633.3316++ | 23 |
| [391, 398] | LLEGEETR (SEQ ID NO: 25) | 473.7456++ | 24 |
| [421, 436] | SAYGGLQTSSYLMSTR (SEQ ID NO: 27) | 861.4118++ | 25 |
| [437, 461] | SFPSYYTSHVQEEQIEVEETIEAAK | 972.1258+++ | 26 |
| | (SEQ ID NO: 28) | | |
| [529, 539] | VEGAGEEQAAK (SEQ ID NO: 29) | 544.7646++ | 27 |

FIG. 41C is a graph summarizing the individual data in FIG. 42B. The y-axis is 14N/15N ratio of the measured peak area for the [529,539] tryptic peptide.

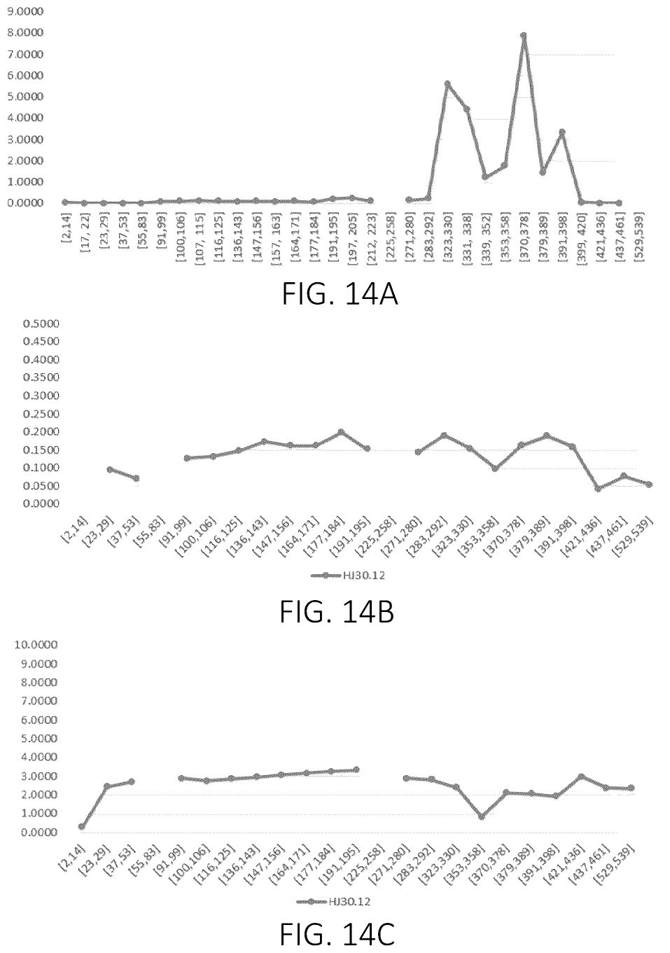

FIG. 42A and FIG. 42B are graphs showing amounts of Nfl enriched from blood as measured by LC-MS in the “30.13 sample”. In each graph, the y-axis is 14N/15N ratio (FIG. 42A) or 14N amount (FIG. 42B) of the measured peak area for each tryptic peptide along the x-axis. When a reliable MS signal was not detected for a given tryptic peptide of the ISTD, no data was reported. Each line is a different blood sample.

FIG. 43A and FIG. 43B are graphs showing amounts of Nfl enriched from blood as measured by LC-MS in the “post30.13_30.4IP sample”. In each graph, the y-axis is 14N/15N ratio (FIG. 43A) or 14N amount (FIG. 43B) of the measured peak area for each tryptic peptide along the x-axis. When a reliable MS signal was not detected for a given tryptic peptide of the ISTD, no data was reported. Each line is a different blood sample.

FIG. 44A and FIG. 44B are graphs showing amounts of Nfl enriched from blood as measured by LC-MS in the “post30.13&4_30.11IP sample”. In each graph, the y-axis is 14N/15N ratio (FIG. 44A) or 14N amount (FIG. 44B) of the measured peak area for each tryptic peptide along the x-axis. When a reliable MS signal was not detected for a given tryptic peptide of the ISTD, no data was reported. Each line is a different blood sample.

FIG. 45 shows immunoprecipitation of recombinant NfL Each of 23 NfL antibodies (HJ.30.x) and one negative control antibody against amyloid beta (HJ5.1) were assessed for their ability to immunoprecipitate full length, recombinant NfL. The relative amounts of recombinant NfL recovery are shown (higher N14/N15=higher recovery of N14. N15 added after IP.

FIG. 46 shows immunoprecipitation of native NfL from pooled CSF Each of the 23 in house NfL antibodies (HJ.30.x) and one negative control antibody against amyloid beta (HJ5.1) were assessed for their ability to immunoprecipitated NfL from CSF. Line colors correspond to individual antibodies and are noted in figure legend. Antibodies appeared to target 3 different regions of NfL.

FIG. 47 shows linearity of quantitative (6 peptide) MS method. Quantitation of all 6 peptides is linear within the tested N14/N15 range.

FIG. 48 shows a map of neurofilament light species indicate that CSF NfL exists as multiple fragment species. Antibodies targeting various domains of NfL used for immunoprecipitation, coupled with mass spectrometry analysis, enabled identification of multiple NfL species in CSF. Light dotted lines represent potential fragments in NfL species identification, while dark solid lines represent identified fragment species. NfL species were identified using 23 different custom antibodies and data used to determine NfL species are shown in FIG. 49A-49B.

FIG. 49A, FIG. 49B and FIG. 49C show brain contains two main NfL species, while CSF has at least 3 main NfL species. Experimental method for sequential IP-MS/MS assay purifying and identifying at least 3 NfL fragment species FIG. 49A Sequential NfL IP from pooled CSF indicates 3 main NfL domains; a mid-domain region from NfL93 to NfL224, another region from NfL324 to NfL359, and a c-terminal region at NfL530 FIG. 49B and brain cortex lysate showing full-length NfL from NfL2 to NfL540, with a c-terminal peptide at NfL 530 FIG. 49C. The blue line depicts peptides identified following the first IP with HJ30.13, the red line depicts peptides identified during the second IP with HJ30.4, and the green line represents peptides identified during the third IP with HJ30.11.

FIG. 50 shows NfL species are increased in AD CSF compared to healthy controls. Sequential IP/MS of the three main CSF NfL species identifies increased NfL levels in Alzheimer's disease dementia (n=4) compared to controls (n=6) for each main species. Red lines represent relative amounts of NfL species for individuals with AD dementia as determined by the presence of amyloid plaques by PET and very mild dementia (CDR=0.5) and black lines represent healthy age matched controls (CDR=0).

FIG. 51A, FIG. 51B, FIG. 51C, FIG. 51D, FIG. 51E, FIG. 51F and FIG. 51G show validation cohort confirms increased NfL324 and NfL530 in AD compared to healthy controls. Schematic showing NfL map and location of peptides in quantitative IP-MS method (FIG. 51A). Comparison of NfL peptides between symptomatic AD participants (N=30; amyloid positive, CDR>0) and healthy controls (N=25; amyloid negative, CDR=0) for Coil 1A and 1B regions NfL101 (FIG. 51B), NfL117 (FIG. 51C) and NfL 165 (FIG. 51D) show non-significant increased trends in AD, no difference in Coil 2B NfL283 region (FIG. 51E), and highly significant increases in NfL323 (FIG. 51F), and c-terminal region NfL529 (FIG. 51G). ** Represents statical significance at p<0.01.

FIG. 52A, FIG. 52B, FIG. 52C, FIG. 52D, FIG. 52E, and FIG. 52F show correlation between IP-MS and ELISA by NfL Species. Correlation between IP-MS and the gold standard Uman Diagnostics ELISA results vary by NfL Species: NfL 101 (FIG. 52A), NfL 117 (FIG. 52B), NfL 165 (FIG. 52C), NfL284 (FIG. 52D), NfL 324 (FIG. 52E), NfL530 (FIG. 52F). There highest correlation is observed between the ELISA and NfL324.

FIG. 53A, FIG. 53B, FIG. 53C, FIG. 53D, FIG. 53E, and FIG. 53F show NfL species correlation with AD dementia stage (CDR sum of boxes). The amount of NfL species are minimally correlated with the stage of dementia severity. The x-axis of each graph denotes the CDR sum of boxes, a clinical scale of dementia with CDR-SB 0 is normal, 0.5 to 6 mild dementia, and >6 moderate clinical dementia. The relative amount of Nfl species is shown in the y-axis as the N14/N15 ratio of the NfL region. Spearman's correlation and p-value calculated for each group: NfL101: Amyloid+spearman r=0.29 (ns, p=0.05), Amyloid-spearman r=0.18 (ns, p=0.30) (FIG. 53A); NfL117: Amyloid+spearman r=0.30 (p=0.04), Amyloid-spearman r=0.18 (ns, p=0.31) (FIG. 53B); NfL 165: Amyloid+spearman r=0.36 (p=0.01), Amyloid-spearman r=0.19 (ns, p=0.28) (FIG. 53C); NfL284: Amyloid+spearman r=0.24 (ns, p=0.10), Amyloid-spearman r=0.31 (ns, p=0.07) (FIG. 53D); NfL324: Amyloid+spearman r=0.30 (p=0.04), Amyloid-spearman r=0.16 (ns, p=0.35) (FIG. 53E); NfL530: Amyloid+spearman r=0.13 (ns, p=0.39), Amyloid-spearman r=0.25 (ns, p=0.14) (FIG. 53F). Participants with amyloid plaques are shown with red circles, and amyloid negative participants are shown with grey squares. NfL101, NfL117, NfL 165, and NfL284 each have n=1 outliers not plotted on graph, but included in calculation of correlation.

FIG. 54A, FIG. 54B, FIG. 54C, FIG. 54D, FIG. 54E, and FIG. 54F show log transformed NfL concentrations by Amyloid status and CDR. Amyloid-CDR 0 group is used as the reference group and the other three groups were compared to the reference group using two sample t tests. P-values were corrected for multiple comparison using Benjamini-Hochberg method. NfL concentrations for each of the four groups (Amyloid negative CDR=0; Amyloid positive CDR=0, Amyloid positive CDR >0, Amyloid negative CDR >0) were compared for NfL 101 (FIG. 54A), NfL 117 (FIG. 54B), NfL 165 (FIG. 54C), NfL 284 (FIG. 54D), NfL 324 (FIG. 54E) and NfL 530 (FIG. 54F).

FIG. 55A, FIG. 55B, FIG. 55C, FIG. 55D, FIG. 55E, and FIG. 55F show Log transformed NfL concentrations by CDR global status. The differences between CDR 0 and CDR >0 groups were compared using two sample t test for NfL 101 (FIG. 55A), NfL 117 (FIG. 55B), NfL 165 (FIG. 55C), NfL 284 (FIG. 55D), NfL 324 (FIG. 55E) and NfL 530 (FIG. 55F).

FIG. 56 shows a heatmap for correlation between NfL species and clinical biomarkers of neurodegeneration, AD, and tau. Map represents Spearman's correlation with darker blue representing stronger correlation and white/light blue representing weak correlation. The strongest correlations of CSF NfL regions were with each other, with NfL324 and NfL530, the c-terminal region, being least correlated with other NfL regions. There were modest correlations between NfL324 or NfL530 and CDR (a clinical dementia rating scale), age, and p-tau and t-tau, while measures of amyloid PET and MMSE had low correlations. Correlations between NfL and tau or ptau were higher for the c-terminal region of NfL (NfL324, NfL530) than for other peptides.

FIG. 57 depicts a schematic of the enrichment process (left side) and a sample type (right side).

FIG. 58A, FIG. 58B, FIG. 58C, FIG. 58D, FIG. 58E, and FIG. 58F show graphs showing the differences in Nfl concentrations for ALS, spinal muscular atrophy (SMA), and control. FIG. 58F shows concentrations of NfL 101. FIG. 58E shows concentrations of NfL 117. FIG. 58C shows concentrations of NfL 165. FIG. 58D shows concentrations of NfL 284. FIG. 58B shows concentrations of NfL 324. FIG. 58A shows concentrations of NfL 530.

FIG. 59A, FIG. 59B, FIG. 59C, FIG. 59D, FIG. 59E, and FIG. 59F show graphs showing the correlation between ALS progression and Nfl concentrations.

FIG. 59F shows the correlation of NfL 101. FIG. 59E shows the correlation of NfL 117.

FIG. 59C shows the correlation of NfL 165. FIG. 59D shows the correlation of NfL 284.

FIG. 59B shows the correlation of NfL 324. FIG. 59A shows the correlation of NfL 530.

FIG. 60A, FIG. 60B, FIG. 60C, FIG. 60D, FIG. 60E, and FIG. 60F show graphs showing the correlation between ALS progression and Nfl concentrations.

FIG. 60F shows the correlation of NfL 101. FIG. 60E shows the correlation of NfL 117.

FIG. 60C shows the correlation of NfL 165. FIG. 60D shows the correlation of NfL 284.

FIG. 60B shows the correlation of NfL 324. FIG. 60A shows the correlation of NfL 530.

FIG. 61 shows sequential IP/MS of CSF NfL species in ALS.

FIG. 62 shows sequential IP/MS of CSF NfL species in control.

FIG. 63 shows sequential IP/MS of CSF NfL species in SMA.

### DETAILED DESCRIPTION

Among the various aspects of the disclosure is the provision of methods to detect and optionally quantify Nfl present in cerebrospinal fluid and blood and use of the methods to detect and optionally measure levels of Nfl biomarkers indicative of neuronal damage. As described in greater detail herein, it has been discovered that certain peptides or parts of Nfl are better indicators of neuronal damage than other peptides or parts of Nfl. Also disclosed herein are a plurality of anti-Nfl epitope binding agents, and their use in the methods of the present disclosure.

### I. Definitions

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

Concentrations, amounts, and other numerical data may be expressed or presented herein in a range format. It is to be understood that such a range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 2 to about 50” should be interpreted to include not only the explicitly recited values of 2 to 50, but also include all individual values and sub-ranges within the indicated range. Thus, included in this numerical range are individual values such as 2, 2.4, 3, 3.7, 4, 5.5, 10, 10.1, 14, 15, 15.98, 20, 20.13, 23, 25.06, 30, 35.1, 38.0, 40, 44, 44.6, 45, 48, and sub-ranges such as from 1-3, from 2-4, from 5-10, from 5-20, from 5-25, from 5-30, from 5-35, from 5-40, from 5-50, from 2-10, from 2-20, from 2-30, from 2-40, from 2-50, etc. This same principle applies to ranges reciting only one numerical value as a minimum or a maximum. Furthermore, such an interpretation should apply regardless of the breadth of the range or the characteristics being described.

The term “about,” as used herein, refers to variation of in the numerical quantity that can occur, for example, through typical measuring techniques and equipment, with respect to any quantifiable variable, including, but not limited to, mass, volume, time, distance, and amount. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations, which can be up to ±5%, but can also be±4%, 3%, 2%, 1%, etc. Whether or not modified by the term “about,” the claims include equivalents to the quantities.

In this disclosure, “comprises,” “comprising,” “containing,” and “having” and the like can have the meaning ascribed to them in U.S. Patent Law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the composition's nature or characteristics would be permissible if present under the “consisting essentially of” language, even though not expressly recited in a list of items following such terminology. In this specification when using an open ended term, like “comprising” or “including,” it is understood that direct support should be afforded also to “consisting essentially of” language as well as “consisting of” language as if stated explicitly and vice versa.

The term “Aβ” refers to peptides derived from a region in the carboxy terminus of a larger protein called amyloid precursor protein (APP). The gene encoding APP is located on chromosome 21. There are many forms of Aβ that may have toxic effects: Aβ peptides are typically 37-43 amino acid sequences long, though they can have truncations and modifications changing their overall size. They can be found in soluble and insoluble compartments, in monomeric, oligomeric and aggregated forms, intracellularly or extracellularly, and may be complexed with other proteins or molecules. The adverse or toxic effects of Aβ may be attributable to any or all of the above noted forms, as well as to others not described specifically. For example, two such Aβ isoforms include Aβ40 and Aβ42; with the Aβ42 isoform being particularly fibrillogenic or insoluble and associated with disease states. The term “Aβ” typically refers to a plurality of Aβ species without discrimination among individual Aβ species. Specific Aβ species are identified by the size of the peptide, e.g., Aβ42, Aβ40, Aβ38 etc.

As used herein, the term “Aβ42/Aβ40 value” means the ratio of the amount of Aβ42 in a sample obtained from a subject compared to the amount of Aβ40 in the same sample.

“Aβ amyloidosis” is defined as clinically abnormal Aβ deposition in the brain. A subject that is determined to have Aβ amyloidosis is referred to herein as “amyloid positive,” while a subject that is determined to not have Aβ amyloidosis is referred to herein as “amyloid negative.” There are accepted indicators of Aβ amyloidosis in the art. At the time of this disclosure, Aβ amyloidosis is directly measured by amyloid imaging (e.g., PiB PET, fluorbetapir, or other imaging methods known in the art) or indirectly measured by decreased cerebrospinal fluid (CSF) Aβ42 or a decreased CSF Aβ42/40 ratio. [11C] PIB-PET imaging with mean cortical binding potential (MCBP) score >0.18 is an indicator of Aβ amyloidosis, as is cerebral spinal fluid (CSF) Aβ42 concentration of about 1 ng/ml measured by immunoprecipitation and mass spectrometry (IP/MS)). Alternatively, a cut-off ratio for CSF Aβ42/40 that maximizes the accuracy in predicting amyloid-positivity as determined by PIB-PET can be used. Values such as these, or others known in the art and/or used in the examples, may be used alone or in combination to clinically confirm Aβ amyloidosis. See, for example, Klunk W E et al. Ann Neurol 55 (3) 2004, Fagan A M et al. Ann Neurol, 2006, 59 (3), Patterson et. al, Annals of Neurology, 2015, 78 (3): 439-453, or Johnson et al., J. Nuc. Med., 2013, 54 (7): 1011-1013, each hereby incorporated by reference in its entirety. Subjects with Aβ amyloidosis may or may not be symptomatic, and symptomatic subjects may or may not satisfy the clinical criteria for a disease associated with Aβ amyloidosis. Non-limiting examples of symptoms associated with Aβ amyloidosis may include impaired cognitive function, altered behavior, abnormal language function, emotional dysregulation, seizures, dementia, and impaired nervous system structure or function. Diseases associated with Aβ amyloidosis include, but are not limited to, Alzheimer's Disease (AD), cerebral amyloid angiopathy (CAA), Lewy body dementia, and inclusion body myositis. Subjects with Aβ amyloidosis are at an increased risk of developing a disease associated with Aβ amyloidosis.

A “clinical sign of Aβ amyloidosis” refers to an objective measure of Aβ deposition known in the art. Clinical signs of Aβ amyloidosis may include, but are not limited to, Aβ deposition identified by amyloid imaging (e.g. PiB PET, fluorbetapir, or other imaging methods known in the art) or by decreased cerebrospinal fluid (CSF) Aβ42 or Aβ42/40 ratio. See, for example, Klunk W E et al. Ann Neurol 55 (3) 2004, and Fagan A M et al. Ann Neurol 59 (3) 2006, each hereby incorporated by reference in its entirety. Clinical signs of Aβ amyloidosis may also include measurements of the metabolism of Aβ, in particular measurements of Aβ42 metabolism alone or in comparison to measurements of the metabolism of other Aβ variants (e.g. Aβ37, Aβ38, Aβ39, Aβ40, and/or total Aβ), as described in U.S. patents Ser. Nos. 14/366,831, 14/523,148 and 14/747,453, each hereby incorporated by reference in its entirety. Additional methods are described in Albert et al. Alzheimer's & Dementia 2007 Vol. 7, pp. 170-179; Mckhann et al., Alzheimer's & Dementia 2007 Vol. 7, pp. 263-269; and Sperling et al. Alzheimer's & Dementia 2007 Vol. 7, pp. 280-292, each hereby incorporated by reference in its entirety. Importantly, a subject with clinical signs of Aβ amyloidosis may or may not have symptoms associated with Aβ deposition. Yet subjects with clinical signs of Aβ amyloidosis are at an increased risk of developing a disease associated with Aβ amyloidosis. Clinical signs of Aβ amyloidosis may also include measurements of other soluble proteins in the CSF or blood, including but not limited to phosphorylated tau species (e.g., tau species phosphorylated at residue T217 and/ro T181, and the like).

A “candidate for amyloid imaging” refers to a subject that has been identified by a clinician as an individual for whom amyloid imaging may be clinically warranted. As a non-limiting example, a candidate for amyloid imaging may be a subject with one or more clinical signs of Aβ amyloidosis, one or more Aβ plaque associated symptoms, one or more CAA associated symptoms, or combinations thereof. A clinician may recommend amyloid imaging for such a subject to direct his or her clinical care. As another non-limiting example, a candidate for amyloid imaging may be a potential participant in a clinical trial for a disease associated with Aβ amyloidosis (either a control subject or a test subject).

An “Aβ plaque associated symptom” or a “CAA associated symptom” refers to any symptom caused by or associated with the formation of amyloid plaques or CAA, respectively, being composed of regularly ordered fibrillar aggregates called amyloid fibrils. Exemplary Aβ plaque associated symptoms may include, but are not limited to, neuronal degeneration, impaired cognitive function, impaired memory, altered behavior, emotional dysregulation, seizures, impaired nervous system structure or function, and an increased risk of development or worsening of Alzheimer's disease or CAA. Neuronal degeneration may include a change in structure of a neuron (including molecular changes such as intracellular accumulation of toxic proteins, protein aggregates, etc. and macro level changes such as change in shape or length of axons or dendrites, change in myelin sheath composition, loss of myelin sheath, etc.), a change in function of a neuron, a loss of function of a neuron, death of a neuron, or any combination thereof. Impaired cognitive function may include but is not limited to difficulties with memory, attention, concentration, language, abstract thought, creativity, executive function, planning, and organization. Altered behavior may include, but is not limited to, physical or verbal aggression, impulsivity, decreased inhibition, apathy, decreased initiation, changes in personality, abuse of alcohol, tobacco or drugs, and other addiction-related behaviors. Emotional dysregulation may include, but is not limited to, depression, anxiety, mania, irritability, and emotional incontinence. Seizures may include but are not limited to generalized tonic-clonic seizures, complex partial seizures, and non-epileptic, psychogenic seizures. Impaired nervous system structure or function may include, but is not limited to, hydrocephalus, Parkinsonism, sleep disorders, psychosis, impairment of balance and coordination. This may include motor impairments such as monoparesis, hemiparesis, tetraparesis, ataxia, ballismus and tremor. This also may include sensory loss or dysfunction including olfactory, tactile, gustatory, visual and auditory sensation. Furthermore, this may include autonomic nervous system impairments such as bowel and bladder dysfunction, sexual dysfunction, blood pressure and temperature dysregulation. Finally, this may include hormonal impairments attributable to dysfunction of the hypothalamus and pituitary gland such as deficiencies and dysregulation of growth hormone, thyroid stimulating hormone, lutenizing hormone, follicle stimulating hormone, gonadotropin releasing hormone, prolactin, and numerous other hormones and modulators.

As used herein, the term “blood sample” refers to a biological sample derived from blood, preferably peripheral (or circulating) blood. The blood sample can be whole blood, plasma or serum, although plasma is typically preferred.

The term “isoform”, as used herein, refers to any of several different forms of the same protein, arising due to alternative splicing of mRNA encoding the protein, post-translational modification of the protein, proteolytic processing of the protein that occurs in vivo, genetic variations and somatic recombination. The terms “isoform,” “species,” and “variant” are used interchangeably (e.g., the term “Nfl isoform” and “Nfl species” may be used interchangeably).

Unless expressly stated otherwise, the term “neurofilament light chain” refers to “human neurofilament light chain” and encompasses all genetically encoded isoforms or variants, as well as species thereof that are C-terminally truncated in vivo, N-terminally truncated in vivo, N-terminally truncated and C-terminally truncated in vivo, post-translationally modified in vivo, or any combination thereof. The terms “neurofilament light chain,” “neurofilament light polypeptide,” and “Nfl” are used interchangeably herein. Full-length Nfl has an amino acid sequence of SEQ ID NO: 1.

| | |
|---|---|
| SEQ ID | MSSFSYEPYYSTSYKRRYVETPRVHISSVRSGYSTARSAYSSYSAPVSSSLS |
| NO: 1 | VRRSYSSSSGSLMPSLENLDLSQVAAISNDLKSIRTQEKAQLQDLNDRFASFI |
| | ERVHELEQQNKVLEAELLVLRQKHSEPSRFRALYEQEIRDLRLAAEDATNEK |
| | QALQGEREGLEETLRNLQARYEEEVLSREDAEGRLMEARKGADEAALARAE |
| | LEKRIDSLMDEISFLKKVHEEEIAELQAQIQYAQISVEMDVTKPDLSAALKDIRA |
| | QYEKLAAKNMQNAEEWFKSRFTVLTESAAKNTDAVRAAKDEVSESRRLLKAK |
| | TLEIEACRGMNEALEKQLQELEDKQNADISAMQDTINKLENELRTTKSEMARY |
| | LKEYQDLLNVKMALDIEIAAYRKLLEGEETRLSFTSVGSITSGYSQSSQVFGRS |
| | AYGGLQTSSYLMSTRSFPSYYTSHVQEEQIEVEETIEAAKAEEAKDEPPSEGE |
| | AEEEEKDKEEAEEEEAAEEEEAAKEESEEAKEEEEGGEGEEGEETKEAEEEE |
| | KKVEGAGEEQAAKKKD |

The term “recombinant Nfl” refers to Nfl encoded by a nucleic acid that has been introduced into a system (e.g., a prokaryotic cell, a eukaryotic cell, or a cell-free expression system) that supports expression of the nucleic acid and its translation into a protein. Methods for producing recombinant proteins are well-known in the art, and the production of recombinant Nfl disclosed herein is not limited to a particular system.

The term “subject” refers to a human, or to a non-human animal expressing human Nfl.

The terms “treat,” “treating,” or “treatment” as used herein, refer to both therapeutic treatment and prophylactic or preventative measures, wherein the object is to prevent or slow down (lessen) an undesired physiological change or disease/disorder. Beneficial or desired clinical results include, but are not limited to, alleviation of symptoms, diminishment of extent of disease, stabilized (i.e., not worsening) state of disease, a delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the disease, condition, or disorder as well as those prone to have the disease, condition or disorder or those in which the disease, condition or disorder is to be prevented.

The term “antibody,” as used herein, is used in the broadest sense and encompasses various antibody and antibody-like structures, including but not limited to full-length monoclonal, polyclonal, and multispecific (e.g., bispecific, trispecific, etc.) antibodies, as well as heavy chain antibodies and antibody fragments provided they exhibit the desired antigen-binding activity. The domain(s) of an antibody that is involved in binding an antigen is referred to as a “variable region” or “variable domain,” and is described in further detail below. A single variable domain may be sufficient to confer antigen-binding specificity. Preferably, but not necessarily, antibodies useful in the discovery are produced recombinantly. Antibodies may or may not be glycosylated, though glycosylated antibodies may be preferred. An “isolated” antibody is one which has been separated from a component of its natural environment. In some embodiments, an antibody is purified to greater than 95% or 99% purity as determined by methods known in the art.

In addition to antibodies described herein, it may be possible to design an antibody mimetic or an aptamer using methods known in the art that functions substantially the same as an antibody of the invention. An “antibody mimetic” refers to a polypeptide or a protein that can specifically bind to an antigen but is not structurally related to an antibody. Antibody mimetics have a mass of about 3 kDa to about 20 kDa. Non-limiting examples of antibody mimetics are affibody molecules, affilins, affimers, alphabodies, anticalins, avimers, DARPins, and monobodies. Aptamers are a class of small nucleic acid ligands that are composed of RNA or single-stranded DNA oligonucleotides and have high specificity and affinity for their targets. Aptamers interact with and bind to their targets through structural recognition, a process similar to that of an antigen-antibody reaction. Aptamers have a lower molecular weight than antibodies, typically about 8-25 kDa.

The terms “full length antibody” and “intact antibody” may be used interchangeably, and refer to an antibody having a structure substantially similar to a native antibody structure or having heavy chains that contain an Fc region as defined herein. The basic structural unit of a native antibody comprises a tetramer. Each tetramer is composed of two identical pairs of polypeptide chains, each pair having one “light” chain (about 25 kDa) and one “heavy” chain (about 50-70 kDa). Light chains are classified as gamma, mu, alpha, and lambda. Heavy chains are classified as gamma, mu, alpha, delta, or epsilon, and define the antibody's isotype as IgG, IgM, IgA, IgD and IgE, respectively. The amino-terminal portion of each light and heavy chain includes a variable region of about 100 to 110 or more amino acid sequences primarily responsible for antigen recognition (VL and VH, respectively). The carboxy-terminal portion of each chain defines a constant region primarily responsible for effector function. Within light and heavy chains, the variable and constant regions are joined by a “J” region of about 12 or more amino acid sequences, with the heavy chain also including a “D” region of about 10 more amino acid sequences. Intact antibodies are properly cross-linked via disulfide bonds, as is known in the art.

The variable domains of the heavy chain and light chain of an antibody generally have similar structures, with each domain comprising four conserved framework regions (FRs) and three hypervariable regions (HVRs). (See, e.g., Kindt et al. Kuby Immunology, 6^{th} ed., W. H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

“Framework region” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence: FR1-HVR1-FR2-HVR2-FR3-HVR3-FR4. The FR domains of a heavy chain and a light chain may differ, as is known in the art.

The term “hypervariable region” or “HVR” as used herein refers to each of the regions of a variable domain which are hypervariable in sequence (also commonly referred to as “complementarity determining regions” or “CDR”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). As used herein, “an HVR derived from a variable region” refers to an HVR that has no more than two amino acid substitutions, as compared to the corresponding HVR from the original variable region. Exemplary HVRs herein include: (a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987)); (b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); (c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262:732-745 (1996)); (d) CDR1-IMGT (positions 27-38), CDR2-IMGT (positions 56-65), and CDR3-IMGT regions (positions 105-116 or 105-117), which are based on IMGT unique numbering (Lefranc, “The IMGT unique numbering for Immunoglobulins, T cell receptors and Ig-like domains,” The Immunologist, 1999, 7:132-136; Lefranc et al., Nucleic Acids Research, 2009, 37 (Database issue): D1006-D1012; Ehrenmann et al., “Chapter 2: Standardized Sequence and Structure Analysis of Antibody Using IMGT,” in Antibody Engineering Volume 2, Eds. Roland E. Kontermann and Stefan Dubel, 2010, Springer-Verlag Berlin Heidelberg, doi: 10.1007/978-3-642-01147-4;

www.imgt.org/IMGTScientificChart/Nomenclature/IMGT-FRCDRdefinition.html), and (e) combinations of (a), (b), (c), and/or (d), as defined below for various antibodies of this disclosure. Unless otherwise indicated, HVR residues and other residues in the variable domain (e.g., FR residues) that are assigned sequence identification numbers are numbered based on IMGT unique numbering, supra.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

A “variant Fc region” comprises an amino acid sequence that can differ from that of a native Fc region by virtue of one or more amino acid substitution(s) and/or by virtue of a modified glycosylation pattern, as compared to a native Fc region or to the Fc region of a parent polypeptide. In an example, a variant Fc region can have from about one to about ten amino acid substitutions, or from about one to about five amino acid substitutions in a native sequence Fc region or in the Fc region of the parent polypeptide. The variant Fc region herein may possess at least about 80% homology, at least about 90% homology, or at least about 95% homology with a native sequence Fc region and/or with an Fc region of a parent polypeptide.

An “antibody fragment” refers to a molecule other than an intact antibody that comprises a portion of an intact antibody that binds the antigen to which the intact antibody binds. Non-limiting examples of antibody fragments include but are not limited to Fv, Fab, Fab′, Fab′-SH, F(ab′)₂; single-chain forms of antibodies and higher order variants thereof; single-domain antibodies, and multispecific antibodies formed from antibody fragments.

Single-chain forms of antibodies, and their higher order forms, may include, but are not limited to, single-domain antibodies, single chain variant fragments (scFvs), divalent scFvs (di-scFvs), trivalent scFvs (tri-scFvs), tetravalent scFvs (tetra-scFvs), diabodies, and triabodies and tetrabodies. ScFv's are comprised of heavy and light chain variable regions connected by a linker. In most instances, but not all, the linker may be a peptide. A linker peptide is preferably from about 5 to 30 amino acids in length, or from about 10 to 25 amino acids in length. Typically, the linker allows for stabilization of the variable domains without interfering with the proper folding and creation of an active binding site. In preferred embodiments, a linker peptide is rich in glycine, as well as serine or threonine. ScFvs can be used to facilitate phage display or can be used for flow cytometry, immunohistochemistry, or as targeting domains. Methods of making and using scFvs are known in the art. ScFvs may also be conjugated to a human constant domain (e.g. a heavy constant domain is derived from an IgG domain, such as IgG1, IgG2, IgG3, or IgG4, or a heavy chain constant domain derived from IgA, IgM, or IgE). Diabodies, triabodies, and tetrabodies and higher order variants are typically created by varying the length of the linker peptide from zero to several amino acids. Alternatively, it is also well known in the art that multivalent binding antibody variants can be generated using self-assembling units linked to the variable domain.

A “single-domain antibody” refers to an antibody fragment consisting of a single, monomeric variable antibody domain.

Multispecific antibodies include bi-specific antibodies, tri-specific, or antibodies of four or more specificities. Multispecific antibodies may be created by combining the heavy and light chains of one antibody with the heavy and light chains of one or more other antibodies. These chains can be covalently linked.

“Monoclonal antibody” refers to an antibody that is derived from a single copy or clone, including e.g., any eukaryotic, prokaryotic, or phage clone. “Monoclonal antibody” is not limited to antibodies produced through hybridoma technology. Monoclonal antibodies can be produced using hybridoma techniques well known in the art, as well as recombinant technologies, phage display technologies, synthetic technologies or combinations of such technologies and other technologies readily known in the art. Furthermore, the monoclonal antibody may be labeled with a detectable label, immobilized on a solid phase and/or conjugated with a heterologous compound (e.g., an enzyme or toxin) according to methods known in the art.

A “heavy chain antibody” refers to an antibody that consists of two heavy chains. A heavy chain antibody may be an IgG-like antibody from camels, llamas, alpacas, sharks, etc., or an IgNAR from a cartiliaginous fish.

A “humanized antibody” refers to a non-human antibody that has been modified to reduce the risk of the non-human antibody eliciting an immune response in humans following administration but retains similar binding specificity and affinity as the starting non-human antibody. A humanized antibody binds to the same or similar epitope as the non-human antibody. The term “humanized antibody” includes an antibody that is composed partially or fully of amino acid sequences derived from a human antibody germline by altering the sequence of an antibody having non-human hypervariable regions (“HVR”). The simplest such alteration may consist simply of substituting the constant region of a human antibody for the murine constant region, thus resulting in a human/murine chimera which may have sufficiently low immunogenicity to be acceptable for pharmaceutical use. Preferably, the variable region of the antibody is also humanized by techniques that are by now well known in the art. For example, the framework regions of a variable region can be substituted by the corresponding human framework regions, while retaining one, several, or all six non-human HVRs. Some framework residues can be substituted with corresponding residues from a non-human VL domain or VH domain (e.g., the non-human antibody from which the HVR residues are derived), e.g., to restore or improve specificity or affinity of the humanized antibody. Substantially human framework regions have at least about 75% homology with a known human framework sequence (i.e. at least about 75%, at least about 80%, at least about 85%, at least about 90%, at least about 95%, or at least about 99% sequence identity). HVRs may also be randomly mutated such that binding activity and affinity for the antigen is maintained or enhanced in the context of fully human germline framework regions or framework regions that are substantially human. As mentioned above, it is sufficient for use in the methods of this discovery to employ an antibody fragment. Further, as used herein, the term “humanized antibody” refers to an antibody comprising a substantially human framework region, at least one HVR from a nonhuman antibody, and in which any constant region present is substantially human. Substantially human constant regions have at least about 90% with a known human constant sequence (i.e. about 90%, about 95%, or about 99% sequence identity). Hence, all parts of a humanized antibody, except possibly the HVRs, are substantially identical to corresponding pairs of one or more germline human immunoglobulin sequences.

If desired, the design of humanized immunoglobulins may be carried out as follows, or using similar methods familiar to those with skill in the art (for example, see Almagro, et al. Front. Biosci. 2008, 13 (5): 1619-33). A murine antibody variable region is aligned to the most similar human germline sequences (e.g. by using BLAST or similar algorithm). The CDR residues from the murine antibody sequence are grafted into the similar human “acceptor” germline. Subsequently, one or more positions near the CDRs or within the framework (e.g., Vernier positions) may be reverted to the original murine amino acid in order to achieve a humanized antibody with similar binding affinity to the original murine antibody. Typically, several versions of humanized antibodies with different reversion mutations are generated and empirically tested for activity. The humanized antibody variant with properties most similar to the parent murine antibody and the fewest murine framework reversions is selected as the final humanized antibody candidate.

The term “epitope-binding agent” refers to an antibody, an aptamer, a nucleic acid, a peptide, a protein, a lipid, a metabolite, a small molecule, or a fragment thereof that recognizes and is capable of specifically binding to a given epitope. The epitope may be a linear epitope or may be a conformational epitope. As used herein, the term “linear epitope” refers to an epitope consisting of a linear (or continuous) sequence of amino acids. The term “conformational epitope” refers to an epitope consisting of discontinuous amino acids on the surface of a protein aggregate that have a specific three-dimensional shape.

As used herein, the term “aptamer” refers to a polynucleotide, generally a RNA or DNA that has a useful biological activity in terms of biochemical activity, molecular recognition or binding attributes. Usually, an aptamer has a molecular activity such as binging to a target molecule at a specific epitope (region). It is generally accepted that an aptamer, which is specific in it binding to a polypeptide, may be synthesized and/or identified by in vitro evolution methods. Means for preparing and characterizing aptamers, including by in vitro evolution methods, are well known in the art. See, for instance U.S. Pat. No. 7,939,313, herein incorporated by reference in its entirety.

### II. Anti-Nfl Epitope-Binding Agents

An “anti-Nfl epitope-binding agent,” as used herein, refers to an isolated epitope-binding agent that binds to recombinant human neurofilament light polypeptide (Nfl) with an affinity constant or affinity of interaction (KD) between about 0.1 μM to about 10 μM, preferably about 0.1 μM to about 1 M, more preferably about 0.1 μM to about 100 nM. Methods for determining the affinity of an epitope-binding agent for an antigen are known in the art, and further illustrated in the Examples. In some embodiments, an anti-Nfl epitiope-binding agent is a nucleic acid aptamer. In some embodiments, an anti-Nfl epitiope-binding agent is an antibody.

Anti-Nfl epitope-binding agents disclosed herein can be described or specified in terms of the epitope(s) that they recognize or bind. The portion of a target polypeptide that specifically interacts with the antigen binding domain of an epitope-binding agent is an “epitope.” Nfl can comprise any number of epitopes, depending on the source of the protein (e.g., recombinant, human), location of the protein (e.g., intracellular, extracellular, brain, CSF, blood, etc.), conformational state, and isoform, etc. Furthermore, it should be noted that an “epitope” on Nfl can be a linear epitope or a conformational epitope, and in both instances can include non-polypeptide elements, e.g., an epitope can include a carbohydrate side chain, a lipid side chain, a phosphate, etc. The term “affinity” refers to a measure of the strength of the binding of an individual epitope with an epitope-binding agent's antigen binding site.

Although anti-Nfl epitope-binding agents of the present disclosure may be generated by using recombinant, full-length Nfl as an antigen, anti-Nfl epitope-binding agents useful for methods of the present disclosure specifically bind to epitopes present on Nfl isolated from blood or plasma. Preferred anti-Nfl epitope-binding agents of the present disclosure specifically bind to epitopes within amino acids 90 to 543 of SEQ ID NO: 1. In various embodiments, anti-Nfl epitope-binding agents of the present disclosure specifically bind to epitopes within amino acids 90 to 300 of SEQ ID NO. 1, or within amino acids 90 to 250 of SEQ ID NO. 1. In other embodiments, anti-Nfl epitope-binding agents of the present disclosure specifically bind to epitopes within amino acids 125 to 300 of SEQ ID NO. 1, within amino acids 125 to 250 of SEQ ID NO. 1, or within amino acids 125 to 200 of SEQ ID NO. 1. In other embodiments, anti-Nfl epitope-binding agents of the present disclosure specifically bind to epitopes within amino acids 251 to 400 of SEQ ID NO. 1, within amino acids 251 to 355 of SEQ ID NO. 1, within amino acids 272 to 355 of SEQ ID NO. 1, or within amino acids 272 to 350 of SEQ ID NO. 1. In other embodiments, anti-Nfl epitope-binding agents of the present disclosure specifically bind to epitopes within amino acids 350 to 543 of SEQ ID NO. 1, within amino acids 397 to 543 of SEQ ID NO. 1, within amino acids 400 to 543 of SEQ ID NO. 1, within amino acids 397 to 540 of SEQ ID NO. 1, or within amino acids 400 to 540 of SEQ ID NO. 1. Methods for epitope mapping are well-known in the art.

In one embodiment, an anti-Nfl epitope-binding agent is HJ30.1, an antigen binding fragment of HJ30.1, or an epitope-binding agent that competitively inhibits HJ30.1 binding to full-length recombinant Nfl. HJ30.1 is a monoclonal antibody produced by hybridoma clone PTA-126966 deposited with the American Type Culture Collection (ATCC).

In one embodiment, an anti-Nfl epitope-binding agent is HJ30.2, an antigen binding fragment of HJ30.2, or an epitope-binding agent that competitively inhibits HJ30.2 binding to full-length recombinant Nfl. HJ30.2 is a monoclonal antibody produced by hybridoma clone PTA-126967 deposited with the ATCC.

In one embodiment, an anti-Nfl epitope-binding agent is HJ30.4, an antigen binding fragment of HJ30.4, or an epitope-binding agent that competitively inhibits HJ30.4 binding to full-length recombinant Nfl. HJ30.4 is a monoclonal antibody produced by hybridoma clone PTA-126968 deposited with the ATCC.

In one embodiment, an anti-Nfl epitope-binding agent is HJ30.7, an antigen binding fragment of HJ30.7, or an epitope-binding agent that competitively inhibits HJ30.7 binding to full-length recombinant Nfl. HJ30.7 is a monoclonal antibody produced by hybridoma clone PTA-126969 deposited with the ATCC.

In one embodiment, an anti-Nfl epitope-binding agent is HJ30.11, an antigen binding fragment of HJ30.11, or an epitope-binding agent that competitively inhibits HJ30.11 binding to full-length recombinant Nfl. HJ30.11 is a monoclonal antibody produced by hybridoma clone PTA-126970 deposited with the ATCC.

In one embodiment, an anti-Nfl epitope-binding agent is HJ30.13, an antigen binding fragment of HJ30.13, or an epitope-binding agent that competitively inhibits HJ30.13 binding to full-length recombinant Nfl. HJ30.13 is a monoclonal antibody produced by hybridoma clone PTA-126971 deposited with the ATCC.

In embodiments where the epitope-binding agent is an antibody, the antibody may or may not have a variant Fc region. In some examples, an Fc region can be modified to have increased or decreased affinity for an Fc receptor on a microglial cell and/or an altered glycosylation pattern. In various embodiments, an anti-Nfl antibody may be a humanized antibody. For instance, is some examples, an anti-Nfl antibody of the present disclosure is a humanized antibody derived from HJ30.1, HJ30.2, HJ30.4, HJ30.7, HJ30.11, or HJ30.13. A humanized anti-Nfl antibody may comprise one or more constant regions, or a portion of a constant region, that is substantially human (i.e. at least 90%, 95%, or 99% sequence identity with a known human framework sequence).

A test epitope-binding agent is said to competitively inhibit binding of a reference epitope-binding agent (e.g., HJ30.1, HJ30.2, HJ30.4, HJ30.7, HJ30.11, HJ30.13, etc.) to a given epitope if the test epitope-binding agent preferentially binds to that epitope to the extent that it blocks binding of the reference epitope-binding agent to the epitope by at least 50%, at least 60%, at least 70%, at least 80%, or at least 90%.

Competitive inhibition can be determined by any method known in the art. In a specific example, competitive inhibition is determined by a competitive inhibition ELISA comprising the steps of: coating a binding surface or support with a purified reference epitope-binding agent to form an epitope-binding agent coated surface; combining a predetermined amount of a purified, labeled antigen and a test sample containing a test epitope-binding agent; adding the incubated mixture of labeled antigen and test epitope-binding agent to said coated surface; incubating said coated surface with said combination of antigen and test epitope-binding agent; and measuring the amount of antigen-binding inhibition as compared to conditions that lack the test epitope-binding agent.

Anti-Nfl epitope-binding agents disclosed herein can also be described or specified in terms of their sequence.

In one embodiment, an anti-Nfl antibody comprises a light chain variable region (VL) that has one or more HVRs derived from HJ30.1 and/or or a heavy chain variable region (VH) that has one or more HVRs derived from HJ30.1. The HVR derived from the VL of HJ30.1 may be L1, L2, L3, or any combination thereof. The HVR derived from the VH of HJ30.1 may be H1, H2, H3, or any combination thereof. The antibody comprising one or more HVRs derived from the VH of HJ30.1 may further comprise VL comprising L1, L2, L3, or any combination thereof of the VL of HJ30.1. In various embodiments of the above, the antibody may be a humanized antibody, or the antibody may have a VL with 90, 91, 92, 93, 94, 95, 96, 97, 98, 99, or 100% identity to the VL of HJ30.1 and/or a VH with 90, 91, 92, 93, 94, 95, 96, 97, 98, 99 or 100% identity to the VH of HJ30.1. In each of the above embodiments, the anti-Nfl antibody may optionally comprise one or more constant regions, or a portion of a constant region, that is substantially human (i.e. at least 90%, 95%, or 99% sequence identity with a known human framework sequence). The present disclosure also encompasses the corresponding nucleic acid sequences, which can readily be determined by one of skill in the art, and may be incorporated into a vector or other large DNA molecule, such as a chromosome, in order to express an antibody of the disclosure.

In one embodiment, an anti-Nfl antibody comprises a light chain variable region (VL) that has one or more HVRs derived from HJ30.2 and/or or a heavy chain variable region (VH) that has one or more HVRs derived from HJ30.2. The HVR derived from the VL of HJ30.2 may be L1, L2, L3, or any combination thereof. The HVR derived from the VH of HJ30.2 may be H1, H2, H3, or any combination thereof. The antibody comprising one or more HVRs derived from the VH of HJ30.2 may further comprise VL comprising L1, L2, L3, or any combination thereof of the VL of HJ30.2. In various embodiments of the above, the antibody may be a humanized antibody, or the antibody may have a VL with 90, 91, 92, 93, 94, 95, 96, 97, 98, 99, or 100% identity to the VL of HJ30.2 and/or a VH with 90, 91, 92, 93, 94, 95, 96, 97, 98, 99 or 100% identity to the VH of HJ30.2. In each of the above embodiments, the anti-Nfl antibody may optionally comprise one or more constant regions, or a portion of a constant region, that is substantially human (i.e. at least 90%, 95%, or 99% sequence identity with a known human framework sequence). The present disclosure also encompasses the corresponding nucleic acid sequences, which can readily be determined by one of skill in the art, and may be incorporated into a vector or other large DNA molecule, such as a chromosome, in order to express an antibody of the disclosure.

In one embodiment, an anti-Nfl antibody comprises a light chain variable region (VL) that has one or more HVRs derived from HJ30.4 and/or or a heavy chain variable region (VH) that has one or more HVRs derived from HJ30.4. The HVR derived from the VL of HJ30.4 may be L1, L2, L3, or any combination thereof. The HVR derived from the VH of HJ30.4 may be H1, H2, H3, or any combination thereof. The antibody comprising one or more HVRs derived from the VH of HJ30.4 may further comprise VL comprising L1, L2, L3, or any combination thereof of the VL of HJ30.4. In various embodiments of the above, the antibody may be a humanized antibody, or the antibody may have a VL with 90, 91, 92, 93, 94, 95, 96, 97, 98, 99, or 100% identity to the VL of HJ30.4 and/or a VH with 90, 91, 92, 93, 94, 95, 96, 97, 98, 99 or 100% identity to the VH of HJ30.4. In each of the above embodiments, the anti-Nfl antibody may optionally comprise one or more constant regions, or a portion of a constant region, that is substantially human (i.e. at least 90%, 95%, or 99% sequence identity with a known human framework sequence). The present disclosure also encompasses the corresponding nucleic acid sequences, which can readily be determined by one of skill in the art, and may be incorporated into a vector or other large DNA molecule, such as a chromosome, in order to express an antibody of the disclosure.

In one embodiment, an anti-Nfl antibody comprises a light chain variable region (VL) that has one or more HVRs derived from HJ30.7 and/or or a heavy chain variable region (VH) that has one or more HVRs derived from HJ30.7. The HVR derived from the VL of HJ30.7 may be L1, L2, L3, or any combination thereof. The HVR derived from the VH of HJ30.7 may be H1, H2, H3, or any combination thereof. The antibody comprising one or more HVRs derived from the VH of HJ30.7 may further comprise VL comprising L1, L2, L3, or any combination thereof of the VL of HJ30.7. In various embodiments of the above, the antibody may be a humanized antibody, or the antibody may have a VL with 90, 91, 92, 93, 94, 95, 96, 97, 98, 99, or 100% identity to the VL of HJ30.7 and/or a VH with 90, 91, 92, 93, 94, 95, 96, 97, 98, 99 or 100% identity to the VH of HJ30.7. In each of the above embodiments, the anti-Nfl antibody may optionally comprise one or more constant regions, or a portion of a constant region, that is substantially human (i.e. at least 90%, 95%, or 99% sequence identity with a known human framework sequence). The present disclosure also encompasses the corresponding nucleic acid sequences, which can readily be determined by one of skill in the art, and may be incorporated into a vector or other large DNA molecule, such as a chromosome, in order to express an antibody of the disclosure.

In one embodiment, an anti-Nfl antibody comprises a light chain variable region (VL) that has one or more HVRs derived from HJ30.11 and/or or a heavy chain variable region (VH) that has one or more HVRs derived from HJ30.11. The HVR derived from the VL of HJ30.11 may be L1, L2, L3, or any combination thereof. The HVR derived from the VH of HJ30.11 may be H1, H2, H3, or any combination thereof. The antibody comprising one or more HVRs derived from the VH of HJ30.11 may further comprise VL comprising L1, L2, L3, or any combination thereof of the VL of HJ30.11. In various embodiments of the above, the antibody may be a humanized antibody, or the antibody may have a VL with 90, 91, 92, 93, 94, 95, 96, 97, 98, 99, or 100% identity to the VL of HJ30.11 and/or a VH with 90, 91, 92, 93, 94, 95, 96, 97, 98, 99 or 100% identity to the VH of HJ30.11. In each of the above embodiments, the anti-Nfl antibody may optionally comprise one or more constant regions, or a portion of a constant region, that is substantially human (i.e. at least 90%, 95%, or 99% sequence identity with a known human framework sequence). The present disclosure also encompasses the corresponding nucleic acid sequences, which can readily be determined by one of skill in the art, and may be incorporated into a vector or other large DNA molecule, such as a chromosome, in order to express an antibody of the disclosure.

In one embodiment, an anti-Nfl antibody comprises a light chain variable region (VL) that has one or more HVRs derived from HJ30.17 and/or or a heavy chain variable region (VH) that has one or more HVRs derived from HJ30.17. The HVR derived from the VL of HJ30.17 may be L1, L2, L3, or any combination thereof. The HVR derived from the VH of HJ30.17 may be H1, H2, H3, or any combination thereof. The antibody comprising one or more HVRs derived from the VH of HJ30.17 may further comprise VL comprising L1, L2, L3, or any combination thereof of the VL of HJ30.17. In various embodiments of the above, the antibody may be a humanized antibody, or the antibody may have a VL with 90, 91, 92, 93, 94, 95, 96, 97, 98, 99, or 100% identity to the VL of HJ30.17 and/or a VH with 90, 91, 92, 93, 94, 95, 96, 97, 98, 99 or 100% identity to the VH of HJ30.17. In each of the above embodiments, the anti-Nfl antibody may optionally comprise one or more constant regions, or a portion of a constant region, that is substantially human (i.e. at least 90%, 95%, or 99% sequence identity with a known human framework sequence). The present disclosure also encompasses the corresponding nucleic acid sequences, which can readily be determined by one of skill in the art, and may be incorporated into a vector or other large DNA molecule, such as a chromosome, in order to express an antibody of the disclosure.

Anti-Nfl epitope-binding agents disclosed herein can also be described or specified in terms of their cross-reactivity. The term “cross-reactivity” refers to the ability of an epitope-binding agent, specific for one antigen, to react with a second antigen; a measure of relatedness between two different antigenic substances. Thus, an epitope-binding agent is cross-reactive if it binds to an epitope other than the one that induced its formation. The cross-reactive epitope generally contains many of the same complementary structural features as the inducing epitope, and in some cases, can actually fit better than the original. For example, certain antibodies have some degree of cross-reactivity, in that they bind related, but non-identical epitopes, e.g., epitopes with at least about 85%, at least about 90%, or at least about 95% identity (as calculated using methods known in the art) to a reference epitope. An antibody, or other epitope-binding agent, can be said to have little or no cross-reactivity if it does not bind epitopes with less than about 95%, less than about 90%, or less than about 85% identity to a reference epitope. An antibody, or other epitope-binding agent, can be deemed “highly specific” for a certain epitope if it does not bind any other analog, ortholog, or homolog of that epitope.

### III. Methods for Detecting Nfl in a Biological Sample

In another aspect, the present disclosure provides a method for detecting Nfl in a biological sample. The method comprises providing a biological sample, enriching for one to a plurality of Nfl isoforms in the biological sample, and detecting one to a plurality of the Nfl isoforms previously enriched. As used herein, the terms “a plurality of Nfl isoforms” and “a population of Nfl isoforms” may be used interchangeably.

### (a) Providing a Biological Sample

Suitable biological samples include a blood sample or a cerebrospinal fluid (CSF) sample obtained from a subject. Blood and CSF contain a plurality of Nfl isoforms, as detailed in the Examples.

The size of the biological sample used may vary depending upon the sample type, the health status of the subject from whom the sample was obtained, and the analytes in addition to Nfl to be analyzed (e.g., Aβ, tau, ApoE, α-synuclein, etc.). CSF sample volumes may be about 0.01 mL to about 5 mL, or about 0.05 mL to about 5 mL. In a specific example, the size of the sample may be about 0.05 mL to about 1 mL CSF. Plasma sample volumes may be about 0.01 mL to about 20 mL, or about 0.1 mL to about 20 mL. In a specific example, the size of the sample may be about 1 mL to about 20 mL blood.

In some embodiments, the subject is a human. A human subject may be waiting for medical care or treatment, may be under medical care or treatment, or may have received medical care or treatment. In various embodiments, a human subject may be a healthy subject, a subject at risk of developing a neurodegenerative disease, a subject with signs and/or symptoms of neuronal damage and/or a neurodegenerative disease, or a subject diagnosed with neuronal damage and/or a neurodegenerative disease. The neurodegenerative disease may be amyotrophic lateral sclerosis, Charcot-Marie-Tooth disease, chronic traumatic encephalopathy (CTE), Creutzfeldt-Jacob disease, Dementia pugilistica, Down's Syndrome, Gerstmann-Sträussler-Scheinker disease, Huntington's disease, inclusion-body myositis, prion protein cerebral amyloid angiopathy, traumatic brain injury (TBI), amyotrophic lateral sclerosis/parkinsonism-dementia complex of Guam, Non-Guamanian motor neuron disease with neurofibrillary tangles, argyrophilic grain dementia, corticobasal degeneration, diffuse neurofibrillary tangles with calcification, frontotetemporal dementia, frontotemporal dementia with parkinsonism linked to chromosome 17, Hallevorden-Spatz disease, Lewy body dementia (LBD), multiple sclerosis, multiple system atrophy, Myotonic dystrophy, Niemann-Pick disease type C, Pallido-ponto-nigral degeneration, Parkinson's disease, Pick's disease, progressive subcortical gliosis, Postencephalitic Parkinsonism, PART (primary age-related Tauopathy), progressive supranuclear palsy, Subacute sclerosing panencephalitis, subacute sclerosis panencephalopathy, Tangle only dementia (or tangle predominant dementia), tangle predominant dementia, white matter tauopathy with globular glial inclusions, mild cognitive impairment (MCI), glaucoma, familial British dementia, familiar Danish dementia, Guadeloupean Parkinsonism, neurodegeneration with brain iron accumulation, SLC9A6-related mental retardation, HIV-related dementia, senile cardiac amyloidosis. A healthy subject, sometimes referred to as a “control subject” or a “healthy control”, minimally has no clinical signs or symptoms of cognitive impairment and may also be “negative” for other clinical signs or symptoms of neuronal damage, neurodegenerative diseases, and/or traumatic brain injury.

In other embodiments, the subject is a laboratory animal. In a further embodiment, the subject is a laboratory animal genetically engineered to express human Nfl.

CSF may have been obtained by lumbar puncture with or without an indwelling CSF catheter. Blood may have been collected by veni-puncture with or without an intravenous catheter, or by a finger stick (or the equivalent thereof). Multiple blood or CSF samples contemporaneously collected from a subject may be pooled to create “a sample”. Once collected, blood or CSF samples may have been processed according to methods known in the art (e.g., centrifugation to remove whole cells and cellular debris; use of additives designed to stabilize and preserve the specimen prior to analytical testing; etc.). Blood or CSF samples may be used immediately or may be frozen and stored indefinitely.

Prior to use in the methods disclosed herein, a biological sample may also have been modified, if needed or desired, to include protease inhibitors, internal standards, detergent(s) and chaotropic agent(s), to deplete other analytes (e.g. proteins, peptides, metabolites, etc.), or any combination thereof.

The term “deplete” means to diminish in quantity or number. Accordingly, a sample depleted of a protein may have any amount of the protein that is measurably less than the amount in the original sample, including no amount of the protein. As a non-limiting example, protein(s) may be depleted from a sample by ultrafiltration or protein precipitation with an acid, an organic solvent or a salt. Generally speaking, these methods are used to reliably reduce high abundance and high molecular weight proteins, which in turn enriches for low molecular weight and/or low abundance proteins and peptides (e.g., tau, Aβ, Nfl, etc.). In a specific example, proteins may be depleted from a sample by precipitation. Briefly, precipitation comprises adding a precipitating agent to a sample and thoroughly mixing, incubating the sample with precipitating agent to precipitate proteins, and separating the precipitated proteins by centrifugation or filtration. The resulting supernatant may then be used in downstream applications. The amount of the reagent needed may be experimentally determined by methods known in the art. Suitable precipitating agents include perchloric acid, trichloroacetic acid, acetonitrile, methanol, and the like. In an exemplary embodiment, proteins are depleted from a sample by acid precipitation. In a further embodiment, proteins are depleted from a sample by acid precipitation using perchloric acid.

In a further example, proteins may be depleted from a sample by acid precipitation using perchloric acid. As used herein, “perchloric acid” refers to 70% perchloric acid unless otherwise indicated. In some embodiments, perchloric acid is added to a final concentration of about 1% v/v to about 15% v/v. In other embodiments, perchloric acid is added to a final concentration of about 1% v/v to about 10% v/v. In other embodiments, perchloric acid is added to a final concentration of about 1% v/v to about 5% v/v. In other embodiments, perchloric acid is added to a final concentration of about 3% v/v to about 15% v/v. In other embodiments, perchloric acid is added to a final concentration of about 3% v/v to about 10% v/v. In other embodiments, perchloric acid is added to a final concentration of about 3% v/v to about 5% v/v. In other embodiments, perchloric acid is added to a final concentration of 3.5% v/v to about 15% v/v, 3.5% v/v to about 10% v/v, or 3.5% v/v to about 5% v/v. In other embodiments, perchloric acid is added to a final concentration of about 3.5% v/v. Following addition of the perchloric acid, the sample is mixed well (e.g., by a vortex mixer) and held at a cold temperature, typically for about 10 minutes or longer, to facilitate precipitation. For example, samples may be held for about 10 minutes to about 60 minutes, about 20 minutes to about 60 minutes, or about 30 minutes to about 60 minutes. In other example, samples may be held for about 15 minutes to about 45 minutes, or about 30 minutes to about 45 minutes. In other examples, samples may be held for about 15 minutes to about 30 minutes, or about 20 minutes to about 40 minutes. In other examples, samples are held for about 30 minutes. The sample is then centrifuged at a cold temperature to pellet the precipitated protein, and the supernatant (i.e., the acid soluble fraction), comprising soluble tau for example, is transferred to a fresh vessel. As used in the above context, a “cold temperature” refers to a temperature of 10° C. or less. For instance, a cold temperature may be about 1° C., about 2° C., about 3° C., about 4° C., about 5° C., about 6° C., about 7° C., about 8° C., about 9° C., or about 10° C. In some embodiments, a narrower temperature range may be preferred, for example, about 3° C. to about 5° C., or even about 4° C. In certain embodiments, a cold temperature may be achieved by placing a sample on ice.

Alternatively, or in addition, protein(s), peptide(s), and/or metabolites may be depleted from a sample by a method that specifically targets the biomolecule of interest, for example by affinity depletion, solid phase extraction, or other method known in the art. Targeted depletion of a protein/peptide, or multiple proteins/peptides, may be used in situations where downstream analysis of that protein/peptide is desired (e.g., identification, quantification, analysis of post-translation modifications, etc.). Typically, at least 50% (e.g., 50%, 60%, 70%, 80%, 90%, or more) of the targeted protein in the starting material is depleted. In some embodiments, about 70% or more, about 80% or more, or about 90% or more of the targeted protein in the starting material is depleted. Affinity depletion refers to methods that deplete a protein of interest from a sample by virtue of its specific binding properties to a molecule. Typically, the molecule is a ligand attached to a solid support, such as a bead, resin, tissue culture plate, etc. (referred to as an immobilized ligand). Immobilization of a ligand to a solid support may also occur after the ligand-protein interaction occurs. Suitable ligands include antibodies, aptamers, and other epitope-binding agents. For instance, Aβ peptides may be identified and quantified by methods known in the art following affinity depletion of Aβ with a suitable epitope-binding agent. Tau may also be similarly identified and quantified. The molecule may also be a polymer or other material that selectively absorbs a protein of interest. As a non-limiting example, polyhydroxymethylene substituted by fat oxethylized alcohol (e.g., PHM-L LIPOSORB, Sigma Aldrich) may be used to selectively absorb lipoproteins (including ApoE) from serum. Identification of the ApoE isoform (“ApoE status”) and/or quantification of ApoE may then occur by methods known in the art. Targeted depletion may also be used to isolate other proteins for subsequent analysis including, but not limited to, apolipoprotein J, synuclein, soluble amyloid precursor protein, alpha-2 macroglobulin, S100B, myelin basic protein, an interleukin, TNF, TREM-2, TDP-43, YKL-40, VILIP-1, prion protein, pNFH, and DJ-1.

### (b) Enriching for One to a Plurality of Nfl Isoforms in the Biological Sample

The term “enrich” means to increase in quantity or number. Blood and CSF contain a plurality of Nfl isoforms. Accordingly, “enriching for one to a plurality of Nfl isoforms in the biological sample” means measurably increasing the amount of the Nfl isoform, or the plurality of Nfl isoforms, per volume of sample as compared to the starting sample (i.e. the biological sample). In some examples, enrichment may be at least about 5-fold. In some examples, enrichment may be about 5-fold to about 1000-fold. For instance, enrichment may be at least about 5-fold, about 10-fold, about 20-fold, about 50-fold, about 100-fold, about 200-fold, about 300-fold, about 400-fold, about 500-fold, about 600-fold, about 700-fold, about 800-fold, about 900-fold, about 1000-fold, or more.

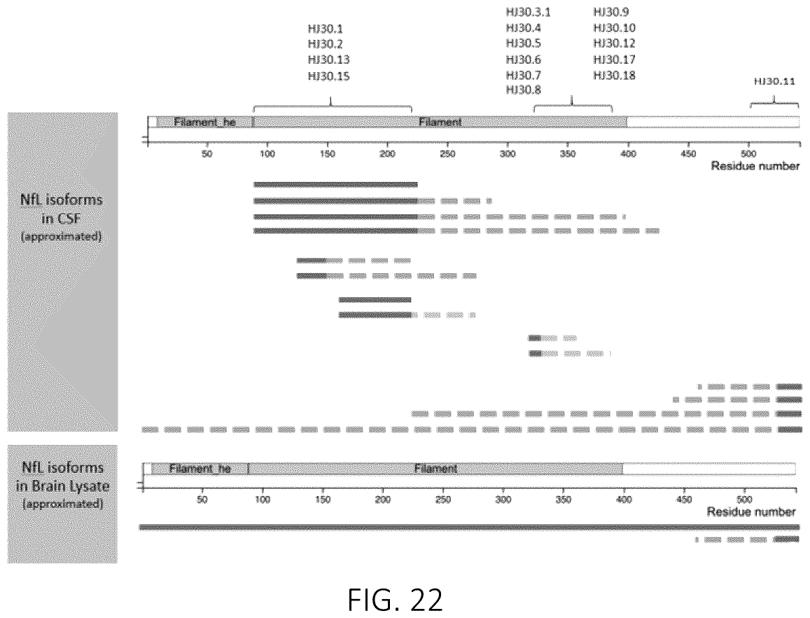

In some embodiments, methods of the present disclosure comprise enriching for one to a plurality of Nfl isoforms in a biological sample, wherein the Nfl isoform(s) are about 350 amino acids in length or less. For instance, the Nfl isoform(s) may be about 300 amino acids in length or less, about 250 amino acids in length or less, about 200 amino acids in length or less, about 150 amino acids in length or less, about 100 amino acids in length or less, or even about 50 amino acids in length or less. Nfl isoforms of about 350 amino acids in length or less may contain an N-terminal truncation, a C-terminal truncation, or an N-terminal truncation and a C-terminal truncation, as to full-length Nfl (which has an amino acid sequence of SEQ ID NO: 1). In further examples, methods of the present disclosure may comprise enriching for one to a plurality of Nfl isoforms that have an amino acid sequence comprising amino acids 92 to 100, amino acids 101 to 107, amino acids 108 to 116, amino acids 117 to 126, amino acids 137 to 144, amino acids 148 to 157, amino acids 158 to 164, amino acids 165 to 172, amino acids 178 to 185, amino acids 192 to 196, or amino acids 198 to 206 of SEQ ID NO: 1, or any combination thereof. In other examples, methods of the present disclosure may comprise enriching for one to a plurality of Nfl isoforms that have an amino acid sequence comprising amino acids 282 to 292, amino acids 323 to 330, amino acids 331 to 338, or amino acids 339 to 353 of SEQ ID NO: 1, or any combination thereof. In still other examples, methods of the present disclosure may comprise enriching for one to a plurality of Nfl isoforms that have an amino acid sequence comprising amino acids 422 to 437, amino acids 438 to 462 and/or amino acids 530 to 540 of SEQ ID NO: 1. In still other examples, methods of the present disclosure may comprise enriching for a first population of Nfl isoforms and then subsequently enriching for a second, third, fourth, or more population(s) of Nfl isoforms, in each instance using the previously depleted sample. In still other examples, methods of the present disclosure may comprise enriching for one or more isoform described in FIG. 22.

Methods of the present disclosure may enrich for a truncated Nfl isoform, or a plurality of truncated Nfl isoforms, by isolating the Nfl isoform(s) from the biological sample (e.g., affinity purification, solid phase extraction, etc.) and/or by removing other Nfl isoforms from the biological sample (e.g., affinity depletion, solid phase extraction etc.). Affinity depletion is described above. Affinity purification refers to methods that enrich for a protein of interest by virtue of its specific binding properties to a molecule. Typically, the molecule is a ligand attached to a solid support, such as a bead, resin, tissue culture plate, etc. (referred to as an immobilized ligand). Immobilization of a ligand to a solid support may also occur after the ligand-protein interaction occurs. Suitable ligands include antibodies, aptamers, and other epitope-binding agents. Purifying Nfl by affinity purification comprises contacting a sample comprising Nfl with a suitable immobilized ligand, one or more wash steps, and elution of Nfl from the immobilized ligand. Reagents for affinity purification of Nfl and/or affinity depletion of Nfl may be generated by methods known in the art (e.g., using full-length recombinant Nfl to generate epitope-binding agents and then selecting epitope-binding agents that bind given epitope(s), using recombinant Nfl peptides to generate epitope-binding agents against certain fragments of Nfl, etc.). Commercially available epitope-binding agents may also be used e.g., ABIN6025698 (Abbexa), ABIN4339158 (Novus Biologicals), 13-0400 (Invitrogen Antibodies), UD1 or UD2 (Uman Diagnostics) etc.). Suitable epitope-binding agents are also described in Section II.

Alternatively, enrichment of an Nfl isoform(s) may not occur directly, but rather amplified detection of an Nfl isoform, or plurality of Nfl isoforms, may occur indirectly. For instance, a proximity ligation assay (e.g., Duo-Link (Sigma Aldrich)) may be used to detect an N-terminal region and C-terminal region of the Nfl isoform with reagents capable of producing an amplified signal when the reagents are bound to the N-terminal region and C-terminal region, respectively. Typically, the reagents are epitope-binding agents. Suitable epitope-binding agents may include those described in Section II and/or commercially available antibodies. Amplified detection of an Nfl isoform, or plurality of Nfl isoforms, by a proximity ligation assay or the like may also occur after an enrichment or depletion step (e.g., after a single enrichment step with an epitope-binding agent that enriches for isoforms comprising amino acids 530 to 540 of SEQ ID NO: 1, etc.).

In a specific embodiment of the above, enriching for one to a plurality of Nfl isoforms in a biological sample may comprise contacting the biological sample with an epitope-binding agent that specifically binds a first population of Nfl isoforms, and isolating the first population of Nfl isoforms, wherein the epitope-binding agent is selected from the group consisting of: (i) an epitope-binding agent that specifically binds to an epitope within amino acids 90 to 250 of SEQ ID NO: 1; (ii) an epitope-binding agent that specifically binds to an epitope within amino acids 116 to 184 of SEQ ID NO: 1; (iii) an epitope-binding agent that specifically binds to an epitope within amino acids 250 to 400 of SEQ ID NO: 1; (iv) an epitope-binding agent that specifically binds to an epitope within amino acids 283 to 338 of SEQ ID NO: 1; (v) an epitope-binding agent that specifically binds to an epitope within amino acids 400 to 543 of SEQ ID NO: 1; (vi) an epitope-binding agent that specifically binds to an epitope within amino acids 437 to 543 of SEQ ID NO: 1; (vii) HJ30.1 or an antigen-binding fragment thereof, HJ30.2 or an antigen-binding fragment thereof, HJ30.13 or an antigen-binding fragment thereof, or an epitope-binding agent that competitively inhibits HJ30.1, HJ30.2 or HJ30.13 binding to full-length, recombinant Nfl; and (vii) HJ30.4 or an antigen-binding fragment thereof, HJ30.7 or an antigen-binding fragment thereof, or an epitope-binding agent that competitively inhibits HJ30.4 or HJ30.7 binding to full-length, recombinant Nfl; and (ix) HJ30.11, an antigen-binding fragment thereof, or an epitope-binding agent that competitively inhibits HJ30.11 binding to full-length, recombinant Nfl.

In another specific embodiment of the above, enriching for one to a plurality of Nfl isoforms in a biological sample may comprise (a) contacting the biological sample with a first epitope-binding agent that specifically binds a first population of Nfl isoforms, and isolating the first population of Nfl isoforms; and (b) contacting a sample depleted of the first population of Nfl isoforms with a second epitope-binding agent that specifically binds a second population of Nfl isoforms, and isolating the second population of Nfl isoforms. Alternatively, the biological sample may be contacted with first epitope-binding agent and the second epitope-bindig agent simultaneously, and the first population of Nfl isoforms and the second population of Nfl isoforms may be isolated sequentially or simultaneously. In preferred embodiments, the second epitope-binding agent binds to an epitope downstream of the first epitope-binding agent's epitope. In one example, the first epitope-binding agent specifically binds to a first epitope within amino acids 1 to 450 of SEQ ID NO: 1; and the second epitope-binding agent specifically binds to an epitope within amino acids 400 to 543 of SEQ ID NO: 1. In another example, the first epitope-binding agent specifically binds to a first epitope within amino acids 90 to 300 of SEQ ID NO: 1 or within amino acids 200 to 400 of SEQ ID NO: 1; and the second epitope-binding agent specifically binds to an epitope within amino acids 400 to 543, or of SEQ ID NO: 1. In another example, the first epitope-binding agent specifically binds to a first epitope within amino acids 90 to 250 of SEQ ID NO: 1 or within amino acids 250 to 400 of SEQ ID NO: 1; and the second epitope is within amino acids 400 to 543 of SEQ ID NO: 1 or within amino acids 430 to 540 of SEQ ID NO: 1. Suitable epitope-binding agents include those described in Section II and/or commercially available antibodies. In a specific embodiment, the first epitope-binding agent may be HJ30.1 or an antigen-binding fragment thereof, HJ30.2 or an antigen-binding fragment thereof, HJ30.13 or an antigen-binding fragment thereof, or an epitope-binding agent that competitively inhibits HJ30.1, HJ30.2 or HJ30.13 binding to full-length, recombinant Nfl. In another specific embodiment, the first epitope-binding agent may be HJ30.4 or an antigen-binding fragment thereof, HJ30.7 or an antigen-binding fragment thereof, or an epitope-binding agent that competitively inhibits HJ30.4 or HJ30.7 binding to full-length, recombinant Nfl. In another specific embodiment, the second epitope-binding agent may be HJ30.11, an antigen-binding fragment thereof, or an epitope-binding agent that competitively inhibits HJ30.11 binding to full-length, recombinant Nfl. In another specific embodiment, the second epitope-binding agent may be HJ30.4 or an antigen-binding fragment thereof, HJ30.7 or an antigen-binding fragment thereof, or an epitope-binding agent that competitively inhibits HJ30.4 or HJ30.7 binding to full-length, recombinant Nfl.

In another specific embodiment of the above, enriching for one to a plurality of Nfl isoforms in a biological sample may comprise (a) contacting the biological sample with a first epitope-binding agent that specifically binds a first population of Nfl isoforms, and isolating the first population of Nfl isoforms; (b) contacting a sample depleted of the first population of Nfl isoforms with a second epitope-binding agent that specifically binds a second population of Nfl isoforms, and isolating the second population of Nfl isoforms; and (c) contacting a sample depleted of the first and second populations of Nfl isoforms with a third epitope-binding agent that specifically binds a third population of Nfl isoforms, and isolating the third population of Nfl isoforms. Alternatively, the biological sample may be contacted with first epitope-binding agent, the second epitope-bindig agent, and the third epitope-bindig agent simultaneously, and the first population of Nfl isoforms, the second population of Nfl isoforms, and the third population of Nfl isoforms may be isolated sequentially or in any combination. In preferred embodiments, the second epitope-binding agent may bind to an epitope downstream of the first epitope-binding agent's epitope, and the third epitope-binding agent may bind to an epitope downstream of the second epitope-binding agent's epitope. Alternatively, the second epitope-binding agent may bind to an epitope upstream of the first epitope-binding agent. In one example, the first epitope-binding agent specifically binds to a first epitope within amino acids 1 to 400 of SEQ ID NO: 1; the second epitope-binding agent specifically binds to a second epitope within amino acids 90 to 450 of SEQ ID NO: 1 that is downstream of the first epitope; and the third epitope-binding agent specifically binds to a third epitope within amino acids 400 to 543 of SEQ ID NO: 1 that is downstream of the second epitope. In another example, the first epitope-binding agent specifically binds to a first epitope within amino acids 90 to 300 of SEQ ID NO: 1; the second epitope-binding agent specifically binds to a second epitope within amino acids 200 to 400 of SEQ ID NO: 1 that is downstream of the first epitope; and the third epitope-binding agent specifically binds to a third epitope within amino acids 400 to 543 of SEQ ID NO: 1 that is downstream of the second epitope. In another example, the first epitope-binding agent specifically binds to a first epitope within amino acids 90 to 250 of SEQ ID NO: 1; the second epitope-binding agent specifically binds to a second epitope within amino acids 250 to 400 of SEQ ID NO: 1 that is downstream of the first epitope; and the third epitope-binding agent specifically binds to a third epitope within amino acids 400 to 543 of SEQ ID NO: 1 that is downstream of the second epitope. Suitable epitope-binding agents include those described in Section II and/or commercially available antibodies. In a specific embodiment, (i) the first epitope-binding agent is HJ30.1 or an antigen-binding fragment thereof, HJ30.2 or an antigen-binding fragment thereof, HJ30.13 or an antigen-binding fragment thereof, or an epitope-binding agent that competitively inhibits HJ30.1, HJ30.2 or HJ30.13 binding to full-length, recombinant Nfl; (ii) the second epitope-binding agent is HJ30.4 or an antigen-binding fragment thereof, HJ30.7 or an antigen-binding fragment thereof, or an epitope-binding agent that competitively inhibits HJ30.4 or HJ30.7 binding to full-length, recombinant Nfl; (iii) the third epitope-binding agent is HJ30.11, an antigen-binding fragment thereof, or an epitope-binding agent that competitively inhibits HJ30.11 binding to full-length, recombinant Nfl; (iv) or any combination thereof.

In another specific embodiment of the above, enriching for one to a plurality of Nfl isoforms in a biological sample may comprise (a) contacting the biological sample with two or more epitope-binding agents simultaneously (not sequentially), wherein each epitope-binding agent specifically binds to a different epitope of Nfl; and (b) detecting and optionally quantifying one to a plurality of Nfl isoforms enriched in step (a). In one example, a first epitope-binding agent specifically binds to a first epitope within amino acids 1 to 400 of SEQ ID NO: 1; a second epitope-binding agent specifically binds to a second epitope within amino acids 90 to 450 of SEQ ID NO: 1 that is downstream of the first epitope; and a third epitope-binding agent specifically binds to a third epitope within amino acids 400 to 543 of SEQ ID NO: 1 that is downstream of the second epitope. In another example, a first epitope-binding agent specifically binds to a first epitope within amino acids 90 to 300 of SEQ ID NO: 1; a second epitope-binding agent specifically binds to a second epitope within amino acids 200 to 400 of SEQ ID NO: 1 that is downstream of the first epitope; and a third epitope-binding agent specifically binds to a third epitope within amino acids 400 to 543 of SEQ ID NO: 1 that is downstream of the second epitope. In another example, a first epitope-binding agent specifically binds to a first epitope within amino acids 90 to 250 of SEQ ID NO: 1; a second epitope-binding agent specifically binds to a second epitope within amino acids 250 to 400 of SEQ ID NO: 1; and a third epitope-binding agent specifically binds to a third epitope within amino acids 400 to 543 of SEQ ID NO: 1. Suitable epitope-binding agents include those described in Section II and/or commercially available antibodies. In a specific embodiment, the two or more epitope-binding agents are selected from the group consisting of HJ30.1, an antigen-binding fragment of HJ30.1, HJ30.2, an antigen-binding fragment of HJ30.2, HJ30.13, an antigen-binding fragment of HJ30.13, HJ30.4, an antigen-binding fragment of HJ30.4, HJ30.7, an antigen-binding fragment of HJ30.7, HJ30.11, an antigen-binding fragment of HJ30.11, or an epitope-binding agent that competitively inhibits HJ30.1, HJ30.2, HJ30.4, HJ30.7, HJ30.11, and HJ30.13 binding to full-length, recombinant Nfl. In another specific embodiment, one epitope-binding agent is selected from group (i), (ii), or (iii), and at least one additional epitope-binding agent is selected from a different of group (i), (ii) or (iii), wherein groups (i), (ii) or (iii) are: (i) HJ30.1 or an antigen-binding fragment thereof, HJ30.2 or an antigen-binding fragment thereof, HJ30.13 or an antigen-binding fragment thereof, or an epitope-binding agent that competitively inhibits HJ30.1, HJ30.2 or HJ30.13 binding to full-length, recombinant Nfl; (ii) HJ30.4 or an antigen-binding fragment thereof, HJ30.7 or an antigen-binding fragment thereof, or an epitope-binding agent that competitively inhibits HJ30.4 or HJ30.7 binding to full-length, recombinant Nfl; (iii) HJ30.11, an antigen-binding fragment thereof, or an epitope-binding agent that competitively inhibits HJ30.11 binding to full-length, recombinant Nfl. In another specific embodiment, at least one epitope-binding agent is selected from each of groups (i), (ii), and (iii), wherein groups (i), (ii) or (iii) are: (i) HJ30.1 or an antigen-binding fragment thereof, HJ30.2 or an antigen-binding fragment thereof, HJ30.13 or an antigen-binding fragment thereof, or an epitope-binding agent that competitively inhibits HJ30.1, HJ30.2 or HJ30.13 binding to full-length, recombinant Nfl; (ii) HJ30.4 or an antigen-binding fragment thereof, HJ30.7 or an antigen-binding fragment thereof, or an epitope-binding agent that competitively inhibits HJ30.4 or HJ30.7 binding to full-length, recombinant Nfl; (iii) HJ30.11, an antigen-binding fragment thereof, or an epitope-binding agent that competitively inhibits HJ30.11 binding to full-length, recombinant Nfl.

An internal standard (abbreviated herein as “ISTD”) may be used to account for variability throughout enrichment and optionally to calculate an absolute concentration. Generally, an internal standard is added before significant sample processing, and it can be added more than once if needed. One or more full-length Nfl isoforms may be used. Alternatively, or in addition, isoforms of Nfl with post-translational modifications and/or peptide fragments of Nfl may also be used. For instance, in embodiments with sequential isolation of multiple isoforms, it may be advantageous to use a number of internal standards equal to the number of isolation steps, wherein each internal standard is a different peptide fragment of Nfl, such that each isolation step will only isolate a single internal standard. In some embodiments, each internal standard may be a different AQUA peptide (i.e., a stable, isotope-labeled peptide corresponding to a peptide of interest to be detected by MS). Alternatively, if Nfl isoforms of the final isolation step are only of interest, a single internal standard may be used, wherein the internal standard is a peptide fragment that will not be isolated/enriched until the final step. In some embodiments, the internal standard may be an AQUA peptide. Typically, internal standards are detectably labeled, so as to differentiate the Nfl standard from endogenous Nfl analyte, but without affecting the chemical properties relied upon for separation. In some embodiments, an internal standard is an isotope-labeled internal Nfl standard. Suitable isotope-labeled internal Nfl standards have a heavy isotope label incorporated into at least one amino acid residue. Generally speaking, the labeled amino acid residues that are incorporated should increase the mass of the peptide without affecting its chemical properties, and the mass shift resulting from the presence of the isotope labels must be sufficient to allow the mass spectrometry method to distinguish the internal standard (IS) from endogenous Nfl analyte signals. As shown herein, suitable heavy isotope labels include, but are not limited to ²H, ¹³C, and ¹⁵N. In one example, an internal standard may be a Lys, Arg, ¹³C and ¹⁵N labeled recombinant, full-length Nfl or peptide fragment of Nfl. Typically, about 0.1-10 ng of internal standard is usually sufficient diluted in an appropriate buffer (e.g., TBEAC, ˜0.01-2% albumin, etc.). In exemplary embodiments, about 1 ng to about 2.5 ng in about 1% human serum albumin (HSA).

### (c) Detecting One to a Plurality of the Nfl Isoforms Previously Enriched

Nfl isoform(s) can be detected, and optionally quantified, in the enriched samples by mass spectrometry, as further detailed below or in the Examples, or by other methods known in the art, including but not limited to an immunoassay, a multiplexed assay (such as xMAP technology by Luminex), a single molecule array assay (such as Simoa® bead technology), a proximity ligation assay (such as DuoLink® by Sigma Aldrich), or the like.

In some embodiments, Nfl isoforms are detected, and optionally quantified, in an enriched sample by mass spectrometry. Briefly, detection by mass spectrometry comprises cleaving the enriched Nfl isoforms with a protease and optionally desalting the resultant cleavage product by solid phase extraction to obtain a sample comprising proteolytic peptides of Nfl; and performing liquid chromatography-mass spectrometry (LC/MS) of the sample comprising proteolytic peptides of Nfl to detect at least one proteolytic peptide of Nfl. In any of the methods disclosed herein, the amount any proteolytic peptide of Nfl may also be quantified (e.g., from the height or integration of the peak in a MS analysis corresponding to the appropriate proteolytic peptide). Thus, in practice, one or more proteolytic peptide of Nfl is used to detect and measure the amount of Nfl protein present in the biological sample. Because blood and CSF contain a plurality of Nfl isoforms, and subsets of the plurality of isoforms share sequence similarity (see FIG. 22), a measurement of a proteolytic peptide of Nfl may describe the level of a plurality of Nfl isoforms in the biological sample that contain the measured proteolytic peptide, unless the enrichment method was specific for a given isoform.

In embodiments where trypsin is the protease, suitable proteolytic peptides of Nfl that indicate the presence of Nfl may be as described in the examples. For instance, tryptic peptides [370,378], [379,389], and [391,398] are not unique to Nfl, and therefore are not preferable when the intent is to quantify Nfl in a biological sample. In some embodiments, suitable proteolytic peptides of Nfl that indicate the presence of Nfl may be selected from the peptides listed in Table C. Exemplary tryptic peptides include the tryptic peptides with amino acids 108-116 of SEQ ID NO: 1, amino acids 117-126 of SEQ ID NO: 1, amino acids 165-172 of SEQ ID NO: 1, amino acids 198-206 of SEQ ID NO: 1, amino acids 324-331 of SEQ ID NO: 1, amino acids 400 to 421 of SEQ ID NO: 1, amino acids 422 to 437 of SEQ ID NO: 1, amino acids 438 to 462 of SEQ ID NO: 1, and amino acids 530-540 of SEQ ID NO: 1. When using an alternative enzyme for digestion, the resulting proteolytic peptides may differ slightly but can be readily determined by a person of ordinary skill in the art. Without wishing to be bound by theory, it is believed that a variation in the amount of a tryptic peptide between two biological samples of the same type (e.g. two blood samples) reflects a difference in the Nfl isoforms that make up those biological samples. As disclosed herein, the amounts of certain proteolytic peptides of Nfl, as well ratios of certain proteolytic peptides of Nfl, may provide clinically meaningful information to diagnose neuronal damage, inform diagnosis of neurodegenerative diseases, select patients for further diagnostic testing, guide treatment decisions, or any combination thereof. Thus, methods that allow for detection and quantification of tryptic peptides of Nfl have utility in the diagnosis, prognosis, and treatment of many diseases.

TABLE C
Suitable tryptic peptides of Nfl

| Tryptic peptide | Amino acid sequence |
|---|---|
| [91, 99] | Amino acids 92-100 of SEQ ID NO: 1 |
| [100, 106] | Amino acids 101-107 of SEQ ID NO: 1 |
| [107, 115] | Amino acids 108-116 of SEQ ID NO: 1 |
| [116, 125] | Amino acids 117-126 of SEQ ID NO: 1 |
| [136, 143] | Amino acids 137-144 of SEQ ID NO: 1 |
| [147, 156] | Amino acids 148-157 of SEQ ID NO: 1 |
| [157, 163] | Amino acids 158-164 of SEQ ID NO: 1 |
| [164, 171] | Amino acids 165-172 of SEQ ID NO: 1 |
| [177, 184] | Amino acids 178-185 of SEQ ID NO: 1 |
| [191, 195] | Amino acids 192-196 of SEQ ID NO: 1 |
| [197, 205] | Amino acids 198-206 of SEQ ID NO: 1 |
| [212, 223] | Amino acids 213-224 of SEQ ID NO: 1 |
| [323, 330] | Amino acids 324-331 of SEQ ID NO: 1 |
| [331, 338] | Amino acids 332-339 of SEQ ID NO: 1 |
| [339, 352] | Amino acids 340-353 of SEQ ID NO: 1 |
| [353, 358] | Amino acids 355-359 of SEQ ID NO: 1 |
| [437, 461] | Amino acids 438-462 of SEQ ID NO: 1 |
| [529, 539] | Amino acids 530-540 of SEQ ID NO: 1 |

Proteolytic peptides of Nfl may be separated by a liquid chromatography system interfaced with a high-resolution mass spectrometer. Suitable LC-MS systems may comprise a<1.0 mm ID column and use a flow rate less than about 100 μl/min. In preferred embodiments, a nanoflow LC-MS system is used (e.g., about 50-100 μm ID column and a flow rate of <1 μL/min, preferably about 100-800 nL/min, more preferably about 200-600 nL/min). In an exemplary embodiment, an LC-MS system may comprise a 0.05 mM ID column and use a flow rate of about 400 nL/min.

Tandem mass spectrometry may be used to improve resolution, as is known in the art, or technology may improve to achieve the resolution of tandem mass spectrometry with a single mass analyzer. Suitable types of mass spectrometers are known in the art. These include, but are not limited to, quadrupole, time-of-flight, ion trap and Orbitrap, as well as hybrid mass spectrometers that combine different types of mass analyzers into one architecture (e.g., Orbitrap Fusion™ Tribrid™ Mass Spectrometer, Orbitrap Fusion™ Lumos™ Mass Spectrometer, Orbitrap Tribrid™ Eclipse™ Mass Spectrometer, Q Exactive Mass Spectrometer, each from ThermoFisher Scientific). In an exemplary embodiment, an LC-MS system may comprise a mass spectrometer selected from Orbitrap Fusion™ Tribrid™ Mass Spectrometer, Orbitrap Fusion™ Lumos™ Mass Spectrometer, Orbitrap Tribrid™ Eclipse™ Mass Spectrometer, or a mass spectrometer with similar or improved ion-focusing and ion-transparency at the quadrupole. Suitable mass spectrometry protocols may be developed by optimizing the number of ions collected prior to analysis (e.g., (AGC setting using an orbitrap) and/or injection time. In an exemplary embodiment, a mass spectrometry protocol outlined in the Examples is used.

Proteolytic peptides analyzed by the MS may be quantified by methods known in the art. Generally speaking, a known amount of an internal standard is added to a sample. The sample is then digested and analyzed by LC-MS. Extracted ion chromatograms are generated for the native peptide and the internal standard. Using peak ratios (e.g., 14N/15N), the quantity of native peptide is calculated.

### IV. Methods for Detecting a Biomarker and Use Thereof

In another aspect, the present disclosure provides a method for detecting, and optionally quantifying, a protein biomarker in a sample obtained from a subject. The method comprises detecting and optionally quantifying Nfl according to a method of Section III, wherein the biological sample is a sample obtained from a subject having or at risk of having neuronal damage, and wherein the biomarker is an enriched Nfl isoform, a ratio of a first Nfl isoform to a second Nfl isoform, an enriched population of Nfl isoforms, or a ratio of a first population of enriched Nfl isoforms and a second population of enriched isoforms. In some embodiment, the neuronal damage may include axonal damage. In some embodiments, the neuronal damage is axonal damage. The type of neuronal damage is not limiting, and may be due to acute or chronic injury (e.g., traumatic brain injury, etc.), neuroinflamamtion, and/or a neurodegenerative disease. Non-limiting examples of neurodegenerative diseases include amyotrophic lateral sclerosis, Charcot-Marie-Tooth disease, chronic traumatic encephalopathy (CTE), Creutzfeldt-Jacob disease, Dementia pugilistica, Down's Syndrome, Gerstmann-Straussler-Scheinker disease, Huntington's disease, inclusion-body myositis, prion protein cerebral amyloid angiopathy, traumatic brain injury, amyotrophic lateral sclerosis/parkinsonism-dementia complex of Guam, Non-Guamanian motor neuron disease with neurofibrillary tangles, argyrophilic grain dementia, corticobasal degeneration, diffuse neurofibrillary tangles with calcification, frontotetemporal dementia, frontotemporal dementia with parkinsonism linked to chromosome 17, Hallevorden-Spatz disease, Lewy body dementia (LBD), multiple sclerosis, multiple system atrophy, Myotonic dystrophy, Niemann-Pick disease type C, Pallido-ponto-nigral degeneration, Parkinson's disease, Pick's disease, progressive subcortical gliosis, Postencephalitic Parkinsonism, PART (primary age-related Tauopathy), progressive supranuclear palsy, Subacute sclerosing panencephalitis, subacute sclerosis panencephalopathy, Tangle only dementia (or tangle predominant dementia), tangle predominant dementia, white matter tauopathy with globular glial inclusions, mild cognitive impairment (MCI), glaucoma, familial British dementia, familiar Danish dementia, Guadeloupean Parkinsonism, neurodegeneration with brain iron accumulation, SLC9A6-related mental retardation, HIV-related dementia, senile cardiac amyloidosis. A healthy subject, sometimes referred to as a “control subject” or a “healthy control”, minimally has no clinical signs or symptoms of cognitive impairment and may also be “negative” for other clinical signs or symptoms of neuronal damage, neurodegenerative diseases, and/or traumatic brain injury.

In some embodiments, the biomarker may be a population of Nfl isoforms selected from the group consisting of isoforms with an amino acid sequence substantially consisting of the rod domain of Nfl, isoforms with an amino acid sequence substantially consisting of a portion of the rod domain of Nfl, isoforms with an amino acid sequence consisting of a portion of the rod-domain of Nfl and no amino acids outside this region, or any combination thereof. The rod domain is typically described as including amino acids 90-396 of SEQ ID NO: 1. For instance, the biomarker may include one or more Nfl isoform comprising amino acids 90 to 396 of SEQ ID NO: 1 that has up to about 330 amino acids in length (e.g., about 330, about 325, about 320, about 315, about 310 amino acids). Alternatively, or in addition, the biomarker may include one or more Nfl isoform that is a fragment of amino acids 90 to 396 of SEQ ID NO: 1. For instance, the biomarker may include one or more Nfl isoform that is less than 310 amino acids in length (about 305, about 300, about 275, about 250, about 225, about 200, about 175, about 150, about 125, about 100, about 75, about 50, about 25, etc.) and comprises amino acids 164 to 171 of SEQ ID NO: 1, amino acids 197 to 205 of SEQ ID NO: 1, amino acids 323 to 330 of SEQ ID NO: 1, or any combination thereof. In some examples, the biomarker may include one or more Nfl isoform that is less than about 200 amino acids in length and comprises amino acids 165 to 172 of SEQ ID NO: 1, amino acids 198 to 206 of SEQ ID NO: 1, or a combination thereof. Alternatively, or in addition, the biomarker may include one or more Nfl isoform that is a fragment of amino acids 125 to 396 of SEQ ID NO: 1. For instance, the biomarker may include one or more Nfl isoform that is about 275 amino acids in length or less, and comprises amino acids 165 to 172 of SEQ ID NO: 1, amino acids 198 to 206 of SEQ ID NO: 1, amino acids 324 to 331 of SEQ ID NO: 1, or any combination thereof.

In some embodiments, the biomarker may be a population of Nfl isoforms selected from the group consisting of isoforms substantially consisting of the tail region of Nfl, isoforms with an amino acid sequence consisting of a portion of the tail region of Nfl, or a combination thereof. The tail region is typically descried as amino acids 397 to 543 of SEQ ID NO: 1. For instance, the biomarker may include one or more Nfl isoform comprising amino acids 397 to 543 of SEQ ID NO: 1 that is 147 amino acids in length to about 200 amino acids in length. Alternatively, or in addition, the biomarker may include one or more Nfl isoform that is a fragment of amino acids 397 to 543 of SEQ ID NO: 1. For instance, the biomarker may include one or more Nfl isoform that is less than 147 amino acids in length and comprises amino acids 422 to 437 of SEQ ID NO: 1, amino acids 438 to 462 of SEQ ID NO: 1, amino acids 530 to 540 of SEQ ID NO: 1, or any combination thereof. In another embodiment, the biomarker may include one or more Nfl isoform that is less about 100 amino acids in length or less and comprises amino acids 422 to 437 of SEQ ID NO: 1, amino acids 438 to 462 of SEQ ID NO: 1, amino acids 530 to 540 of SEQ ID NO: 1, or any combination thereof.

In further embodiments, the biomarker may be a ratio of two biomarkers described above. As non-limiting examples, the biomarker may be a ratio of two biomarkers from the rod region, or a ratio of two biomarkers from the tail region, or more preferably a ratio of one biomarker from the rod region and one biomarker from the tail region. In still further embodiments, the biomarker may be a ratio of a biomarker to the total amount of Nfl. Other mathematical operations, and the use of more than two biomarkers, are also contemplated. For example, when a first and second biological sample are analyzed where the second sample is obtained a period of time after the first (e.g. days, weeks, months, years) the rate of change of a biomarker may be used.

In some embodiments, the biomarker may be a population of Nfl isoforms affinity purified with an epitope-binding agent chosen from (i) HJ30.1 or an antigen-binding fragment thereof, HJ30.2 or an antigen-binding fragment thereof, HJ30.13 or an antigen-binding fragment thereof, or an epitope-binding agent that competitively inhibits HJ30.1, HJ30.2 or HJ30.13 binding to full-length, recombinant Nfl; (ii) HJ30.4 or an antigen-binding fragment thereof, HJ30.7 or an antigen-binding fragment thereof, or an epitope-binding agent that competitively inhibits HJ30.4 or HJ30.7 binding to full-length, recombinant Nfl; or (iii) HJ30.11, an antigen-binding fragment thereof, or an epitope-binding agent that competitively inhibits HJ30.11 binding to full-length, recombinant Nfl.

In some embodiments, the biomarker may be:
(a) a population of Nfl isoforms comprising amino acids 165 to 172 of SEQ ID NO: 1 and/or amino acids 198 to 206 of SEQ ID NO: 1, wherein the population of Nfl isoforms is affinity purified with an epitope-binding agent chosen from (i) HJ30.1 or an antigen-binding fragment thereof, HJ30.2 or an antigen-binding fragment thereof, HJ30.13 or an antigen-binding fragment thereof, or an epitope-binding agent that competitively inhibits HJ30.1, HJ30.2 or HJ30.13 binding to full-length, recombinant Nfl;
(b) a population of Nfl isoforms comprising amino acids 324 to 331 of SEQ ID NO: 1, wherein the population of Nfl isoforms is affinity purified with an epitope-binding agent chosen from HJ30.4 or an antigen-binding fragment thereof, HJ30.7 or an antigen-binding fragment thereof, or an epitope-binding agent that competitively inhibits HJ30.4 or HJ30.7 binding to full-length, recombinant Nfl;
(c) a population of Nfl isoforms comprising amino acids 530 to 540 of SEQ ID NO: 1, wherein the population of Nfl isoforms is affinity purified with an epitope-binding agent chosen from HJ30.11, an antigen-binding fragment thereof, or an epitope-binding agent that competitively inhibits HJ30.11 binding to full-length, recombinant Nfl;
(d) a ratio of (a) to (b);
(e) a ratio of (b) to (c); or
(f) a ratio of (a) to (c).

In the above embodiments, each population of Nfl isoforms may be isolated from the biological sample or may be isolated from a sample previously depleted of other Nfl isoforms. As a non-limiting example, the population of Nfl isoforms in (c) may be isolated from a sample depleted of (a), depleted of (b), or depleted of (a) and (b).

In one example, the method comprises (a) providing a biological sample obtained from the subject, wherein the biological sample is a blood sample or a CSF sample, (b) enriching for one to a plurality of Nfl isoforms in the biological sample, wherein enriching comprises/consists of (i) contacting the biological sample with a first epitope-binding agent that specifically binds a first population of Nfl isoforms, and isolating the first population of Nfl isoforms; (ii) contacting a sample depleted of the first population of Nfl isoforms with a second epitope-binding agent that specifically binds a second population of Nfl isoforms, and isolating the second population of Nfl isoforms; and (iii) contacting a sample depleted of the first and second populations of Nfl isoforms with a third epitope-binding agent that specifically binds a third population of Nfl isoforms, and isolating the third population of Nfl isoforms; and (c) detecting and optionally quantifying one to a plurality of Nfl isoforms in the first population, second population, third population, or any combination thereof, wherein the biomarker is the Nfl isoform or population of Nfl isoforms detected in (c), or a ratio between Nfl isoforms or population of Nfl isoforms detected in (c). In preferred embodiments, the second epitope-binding agent may bind to an epitope downstream of the first epitope-binding agent's epitope, and the third epitope-binding agent may bind to an epitope downstream of the second epitope-binding agent's epitope. Alternatively, the second epitope-binding agent may bind to an epitope upstream of the first epitope-binding agent. In one example, the first epitope-binding agent specifically binds to a first epitope within amino acids 1 to 400 of SEQ ID NO: 1; the second epitope-binding agent specifically binds to a second epitope within amino acids 90 to 450 of SEQ ID NO: 1; and the third epitope-binding agent specifically binds to a third epitope within amino acids 400 to 543 of SEQ ID NO: 1. In another example, the first epitope-binding agent specifically binds to a first epitope within amino acids 90 to 300 of SEQ ID NO: 1; the second epitope-binding agent specifically binds to a second epitope within amino acids 200 to 400 of SEQ ID NO: 1 that is downstream of the first epitope; and the third epitope-binding agent specifically binds to a third epitope within amino acids 400 to 543 of SEQ ID NO: 1 that is downstream of the second epitope. In another example, the first epitope-binding agent specifically binds to a first epitope within amino acids 90 to 250 of SEQ ID NO: 1; the second epitope-binding agent specifically binds to a second epitope within amino acids 250 to 400 of SEQ ID NO: 1; and the third epitope-binding agent specifically binds to a third epitope within amino acids 400 to 543 of SEQ ID NO: 1. Suitable epitope-binding agents include those described in Section II and/or commercially available antibodies. In a specific embodiment, (i) the first epitope-binding agent is HJ30.1 or an antigen-binding fragment thereof, HJ30.2 or an antigen-binding fragment thereof, HJ30.13 or an antigen-binding fragment thereof, or an epitope-binding agent that competitively inhibits HJ30.1, HJ30.2 or HJ30.13 binding to full-length, recombinant Nfl; (ii) the second epitope-binding agent is HJ30.4 or an antigen-binding fragment thereof, HJ30.7 or an antigen-binding fragment thereof, or an epitope-binding agent that competitively inhibits HJ30.4 or HJ30.7 binding to full-length, recombinant Nfl; (iii) the third epitope-binding agent is HJ30.11, an antigen-binding fragment thereof, or an epitope-binding agent that competitively inhibits HJ30.11 binding to full-length, recombinant Nfl; (iv) or any combination thereof.

In some embodiments, the method comprises (a) providing a biological sample obtained from the subject, wherein the biological sample is a blood sample or a CSF sample, (b) enriching for a plurality of Nfl isoforms by contacting the biological sample with two or more epitope-binding agents, wherein each epitope-binding agent specifically binds to a different epitope of Nfl; and (c) detecting and optionally quantifying one to a plurality of Nfl isoforms enriched in step (b), wherein the biomarker is an Nfl isoform or population of Nfl isoforms detected in (c), or a ratio between Nfl isoforms or population of Nfl isoforms detected in (c). In one example, a first epitope-binding agent specifically binds to a first epitope within amino acids 1 to 400 of SEQ ID NO: 1; a second epitope-binding agent specifically binds to a second epitope within amino acids 90 to 450 of SEQ ID NO: 1 that is downstream of the first epitope; and a third epitope-binding agent specifically binds to a third epitope within amino acids 400 to 543 of SEQ ID NO: 1 that is downstream of the second epitope. In another example, a first epitope-binding agent specifically binds to a first epitope within amino acids 90 to 300 of SEQ ID NO: 1; a second epitope-binding agent specifically binds to a second epitope within amino acids 200 to 400 of SEQ ID NO: 1 that is downstream of the first epitope; and a third epitope-binding agent specifically binds to a third epitope within amino acids 400 to 543 of SEQ ID NO: 1 that is downstream of the second epitope. In another example, a first epitope-binding agent specifically binds to a first epitope within amino acids 90 to 250 of SEQ ID NO: 1; a second epitope-binding agent specifically binds to a second epitope within amino acids 250 to 400 of SEQ ID NO: 1; and a third epitope-binding agent specifically binds to a third epitope within amino acids 400 to 543 of SEQ ID NO: 1. Suitable epitope-binding agents include those described in Section II and/or commercially available antibodies. In a specific embodiment, the two or more epitope-binding agents are selected from the group consisting of HJ30.1, an antigen-binding fragment of HJ30.1, HJ30.2, an antigen-binding fragment of HJ30.2, HJ30.13, an antigen-binding fragment of HJ30.13, HJ30.4, an antigen-binding fragment of HJ30.4, HJ30.7, an antigen-binding fragment of HJ30.7, HJ30.11, an antigen-binding fragment of HJ30.11, or an epitope-binding agent that competitively inhibits HJ30.1, HJ30.2, HJ30.4, HJ30.7, HJ30.11, and HJ30.13 binding to full-length, recombinant Nfl. In another specific embodiment, one epitope-binding agent is selected from group (i), (ii), or (iii), and at least one additional epitope-binding agent is selected from a different of group (i), (ii) or (iii), wherein groups (i), (ii) or (iii) are: (i) HJ30.1 or an antigen-binding fragment thereof, HJ30.2 or an antigen-binding fragment thereof, HJ30.13 or an antigen-binding fragment thereof, or an epitope-binding agent that competitively inhibits HJ30.1, HJ30.2 or HJ30.13 binding to full-length, recombinant Nfl; (ii) HJ30.4 or an antigen-binding fragment thereof, HJ30.7 or an antigen-binding fragment thereof, or an epitope-binding agent that competitively inhibits HJ30.4 or HJ30.7 binding to full-length, recombinant Nfl; (iii) HJ30.11, an antigen-binding fragment thereof, or an epitope-binding agent that competitively inhibits HJ30.11 binding to full-length, recombinant Nfl. In another specific embodiment, at least one epitope-binding agent is selected from each of groups (i), (ii), and (iii), wherein groups (i), (ii) or (iii) are: (i) HJ30.1 or an antigen-binding fragment thereof, HJ30.2 or an antigen-binding fragment thereof, HJ30.13 or an antigen-binding fragment thereof, or an epitope-binding agent that competitively inhibits HJ30.1, HJ30.2 or HJ30.13 binding to full-length, recombinant Nfl; (ii) HJ30.4 or an antigen-binding fragment thereof, HJ30.7 or an antigen-binding fragment thereof, or an epitope-binding agent that competitively inhibits HJ30.4 or HJ30.7 binding to full-length, recombinant Nfl; (iii) HJ30.11, an antigen-binding fragment thereof, or an epitope-binding agent that competitively inhibits HJ30.11 binding to full-length, recombinant Nfl.

Detection and quantification of the biomarker may be used for a number of purposes. Non-limiting examples include diagnosing neuronal damage, diagnosing a disease or condition characterized by neuronal damage (e.g., traumatic brain injury, a neurodegenerative disease, etc.), monitoring/measuring the development or progression of neuronal damage and/or a disease state characterized by neuronal damage, treating a subject with neuronal damage, determining/measuring the efficacy of a given treatment, and the like.

Accordingly, in another aspect, the method comprises detecting and quantifying the level of a biomarker, as described in any of the embodiments above, and determining if the level is reduced in comparison to its level in control subjects who are cognitively normal. In another aspect, the method comprises detecting and quantifying the level of a biomarker, as described in any of the embodiments above, and determining if the level is reduced in comparison to its level in control subjects who are cognitively normal and also negative for one or more additional clinical sign or symptom of a neurodegenerative disease or traumatic brain injury. In another aspect, the method comprises detecting and quantifying the level of a biomarker, as described in any of the embodiments above, and determining if the level is reduced in comparison to its level in control subjects who are cognitively normal and also amyloid negative (Aβ-). In another aspect, the method comprises detecting and quantifying the level of a biomarker, as described in any of the embodiments above, and determining if the level is reduced in comparison to its level in control subjects who are cognitively normal and also negative for pathological levels of tau deposits in the brain. Clinical tests for evaluating cognitive impairment, including dementia, are known in the art. As a non-limiting example, the Clinical Dementia Rating (CDR) test may be used. The use of tau PET tracers or Aβ PET tracers to assess pathological protein deposition in the brain are also well known in the art.

In some embodiments, a subject may be diagnosed as having neuronal damage when the level of the biomarker significantly deviates from the mean in the control subjects. “Significantly deviates from the mean” refers to values that are at least 1 standard deviation, preferably at least 1.3 standard deviations, more preferably at least 1.5 standard deviations or even more preferably at least 2 standard deviations, above or below the mean (e.g., 1σ, 1.1σ, 1.2σ, 1.3σ, 1.4σ, 1.5σ, etc., where σ is the standard deviation defined by the normal distribution measured in a control population). In addition to using a threshold (e.g., at least 1 standard deviation above or below the mean), in some embodiments the extent of change above or below the mean may be used to diagnose a subject.

In another aspect, the method comprises detecting and quantifying the level of a biomarker, as described in any of the embodiments above, in a first biological sample obtained from the subject and a second biological sample obtained from the subject, wherein the first biological sample and the second biological sample are both a blood sample or both a CSF sample, and wherein the second biological sample was obtained after the first biological sample.

In another aspect, the method comprises detecting and quantifying the level of a biomarker, as described in any of the embodiments above, in a first biological sample obtained from the subject and a second biological sample obtained from the subject, wherein the first biological sample and the second biological sample are both a blood sample or both a CSF sample, and wherein the second biological sample was obtained after the first biological sample. An increase in the level of biomarker in the second sample as compared to the first sample indicates an increase in neuronal damage. In some embodiments, the rate of change in the levels of the biomarker between the first and subsequent samples is used to determine the stage of disease and/or extent of neuronal damage. Accordingly, such methods may be used to monitor a subject has neuronal damage or is at risk of having neuronal damage.

In some embodiments, one or more of the above methods may be used in combination with one or more disease biomarker known in the art to diagnose, stage, and/or treat specific neurodegenerative diseases. For instance, data show that a pathophysiological cascade of events in AD progression begins with altered CSF and blood plasma Aβ42/Aβ40 ratio, followed by increases in amyloid plaques as measured by amyloid PET, associated with increased phosphorylation of specific CSF tau species (e.g., p-tau217, p-tau231, p-tau181, p-tau153, p-tau111), before increases in p-tau205, NfL, total tau concentrations, hypometabolism, and atrophy. Finally, some species of MTBR-tau (e.g., MTBR-tau299, MTBR-tau243, MTBR-tau354, MTBR-tau3R) increase before or with tau aggregation by tau PET and onset of clinical symptoms and correlate with longitudinal tau aggregation and clinical progression. These findings indicate that CSF and blood Nfl measures, in combination with one or more of Aβ, p-tau, MTBR-tau measures are highly precise biomarkers of brain amyloidosis, tauopathy, and neurodegeneration and can accurately identify stages of preclinical and clinical AD and predict the effectiveness of treatments targeting certain disease processes. Combination use with other disease biomarkers for the diagnosis, staging, and treatment of neurodegenerative diseases other than AD are also contemplated.

In some embodiments, one or more of the above methods may be used in addition to, or as an alternative to, a more invasive diagnostic method, such as PET or lumbar puncture. Thus, in any of the above methods, the subject in some aspects has not received previous diagnostic testing such as PET imaging.

In some embodiments, one or more of the above methods may be used determine whether a subject should receive additional diagnostic testing, which may, for instance, be a more invasive diagnostic method. For example, if Nfl levels, or Nfl levels in combination with another clinical sign, suggest Alzheimer's disease, the subject may be selected to receive amyloid-based PET or tau-based PET. Alternatively, or in addition, Nfl levels may be used to select a subject to receive cognitive testing or further cognitive testing if initial tests have been performed, for example to further confirm presence of disease or a particular disease stage. Alternatively, or in addition, Nfl levels may be used to select a subject to receive additional biomarker testing, such as testing to determine total levels, or levels of specific isoforms, of apolipoprotein J, huntingtin protein, synuclein, soluble amyloid precursor protein, alpha-2 macroglobulin, S100B, myelin basic protein, an interleukin, superoxide dismutase, TNF, TREM-2, TDP-43, YKL-40, VILIP-1, prion protein, pNFH, DJ-1, and the like.

In some embodiments, one or more of the above methods may also be used to prognose cognitive status or cognitive decline. Thus, in some embodiments, the disclosure relates to the above methods for use in predicting cognitive status or predicting cognitive decline in a human subject with neuronal damage, including neuronal damage caused by a neurodegenerative disease, neuroinflammation, or traumatic brain injury. Accordingly, in further embodiments, the disclosure relates to the above methods for use in predicting neurodegenerative disease progression.

In another aspect, the method comprises (a) detecting and quantifying the level of a biomarker, as described in any of the embodiments above, in a first biological sample obtained from the subject, a biomarker as described herein; (b) administering a treatment to the subject; and (c) detecting and quantifying, in a second biological sample obtained from the subject after the treatment, the biomarker quantified in step (a); wherein the first biological sample and the second biological sample are both a blood sample or both a CSF sample. Either no change in the level of the biomarker, or a decrease in the level of the biomarker, in the second sample as compared to the first sample indicates a positive treatment response. In addition, an increase in the level of the biomarker in the second sample as compared to the first sample may also indicate a positive treatment response when the increase is less than an increase that occurs in a control group of subjects that have neuronal damage but were not administered treatment. Preferably the control subjects have neuronal damage due to the same disease process or type of injury. Accordingly, such methods may be used to measure a treatment response in a subject having or at risk of having neuronal damage.

In another aspect, the present disclosure comprises treating a subject diagnosed with a neuronal damage or at risk of having neuronal damage. The method comprises (a) quantifying, in a sample obtained from the subject, a biomarker as described herein; and (b) administering to the subject a pharmaceutical composition to decrease or stabilize the amount of the biomarker measured in step (a).

### V. Compositions

In another aspect, the present disclosure also provides compositions comprising a biomarker of Section IV and an internal standard.

In another aspect, the present disclosure provides compositions comprising an internal standard and a plurality of Nfl isoforms selected from the group consisting of isoforms with an amino acid sequence substantially consisting of the rod domain of Nfl (typically described as amino acids 90-396 of SEQ ID NO: 1), isoforms with an amino acid sequence substantially consisting of a portion of the rod domain of Nfl, isoforms with an amino acid sequence consisting of a portion of the rod-domain of Nfl and no amino acids outside this region, or any combination thereof. For instance, the composition may include one or more Nfl isoform comprising amino acids 90 to 396 of SEQ ID NO: 1 that has up to about 330 amino acids in length. Alternatively, or in addition, the composition may include one or more Nfl isoform that is a fragment of amino acids 90 to 396 of SEQ ID NO: 1. For instance, the composition may include one or more Nfl isoform that is less than 310 amino acids in length (about 305, about 300, about 275, about 250, about 225, about 200, about 175, about 150, about 125, about 100, about 75, about 50, about 25, etc.) and comprises amino acids 165 to 172 of SEQ ID NO: 1, amino acids 198 to 206 of SEQ ID NO: 1, amino acids 324 to 331 of SEQ ID NO: 1, or any combination thereof. In some examples, the composition may include one or more Nfl isoform that is less than about 200 amino acids in length and comprises amino acids 165 to 172 of SEQ ID NO: 1, amino acids 198 to 206 of SEQ ID NO: 1, or a combination thereof. Alternatively, or in addition, the composition may include one or more Nfl isoform that is a fragment of amino acids 125 to 396 of SEQ ID NO: 1. For instance, the composition may include one or more Nfl isoform that is about 275 amino acids in length or less, and comprises amino acids 165 to 172 of SEQ ID NO: 1, amino acids 198 to 206 of SEQ ID NO: 1, amino acids 324 to 331 of SEQ ID NO: 1, or any combination thereof.

In another aspect, the present disclosure provides compositions comprising an internal standard and a plurality of Nfl isoforms selected from the group consisting of isoforms substantially consisting of the tail region of Nfl, isoforms with an amino acid sequence consisting of a portion of the tail region of Nfl, or a combination thereof. The tail region is typically descried as amino acids 397 to 543 of SEQ ID NO: 1. For instance, the composition may include one or more Nfl isoform comprising amino acids 397 to 543 of SEQ ID NO: 1 that is 147 amino acids in length to about 200 amino acids in length. Alternatively, or in addition, the composition may include one or more Nfl isoform that is a fragment of amino acids 397 to 543 of SEQ ID NO: 1. For instance, the composition may include one or more Nfl isoform that is less than 147 amino acids in length and comprises amino acids 422 to 437 of SEQ ID NO: 1, amino acids 438 to 462 of SEQ ID NO: 1, amino acids 530 to 540 of SEQ ID NO: 1, or any combination thereof. In another embodiment, the composition may include one or more Nfl isoform that is less about 100 amino acids in length or less and comprises amino acids 422 to 437 of SEQ ID NO: 1, amino acids 438 to 462 of SEQ ID NO: 1, amino acids 530 to 540 of SEQ ID NO: 1, or any combination thereof.

In another aspect, the present disclosure provides compositions comprising an internal standard and a peptide selected from the group consisting of a peptide consisting of amino acids 165 to 172 of SEQ ID NO: 1, a peptide consisting of amino acids 198 to 206 of SEQ ID NO: 1, a peptide consisting of amino acids 324 to 331 of SEQ ID NO: 1, and a peptide consisting of amino acids 530 to 540 of SEQ ID NO: 1. In another aspect, the present disclosure provides compositions comprising an internal standard and a peptide consisting of amino acids 530 to 540 of SEQ ID NO: 1, wherein the composition contains negligible amounts of a peptide consisting of amino acids 165 to 172 of SEQ ID NO: 1, a peptide consisting of amino acids 198 to 206 of SEQ ID NO: 1, and a peptide consisting of amino acids 324 to 331 of SEQ ID NO: 1.

In some embodiments of the above compositions, the internal standard is recombinant Nfl, or a fragment thereof, that is detectably labeled. In some embodiments, the internal standard is detectably labeled with a heavy isotope label selected from 2H, 13C, and 15N. In some embodiments, the internal standard is an AQUA peptide. In particular embodiments, the internal standard that is detectably labeled with a heavy isotope label and the biomarker or plurality of Nfl isoforms or peptide may be in a ratio of that more than 0.01 to 1, respectively and less than 1 to 100, respectively; or may be in a ratio that is about 0.1 to 1, respectively, to about 10 to 1, respectively.

The amount of peptide or the amount of biomarker or the amount of the plurality of Nfl is isoforms is at least 0.1 pg. In some embodiments, the amount of peptide, biomarker, or plurality of Nfl isoforms is about 0.1 pg to about 10,000 ng, or about 0.1 pg to about 5,000 ng, or about 0.1 pg to about 1,000 ng. In some embodiments, the amount of peptide, biomarker, or plurality of Nfl isoforms is about 1 pg to about 10,000 ng, or about 1 pg to about 5,000 ng, or about 1 pg to about 1,000 ng. In some embodiments, the amount of peptide, biomarker, or plurality of Nfl isoforms is about 10 pg to about 10,000 ng, or about 10 pg to about 5,000 ng, or about 10 pg to about 1,000 ng. In some embodiments, the amount of peptide, biomarker, or plurality of Nfl isoforms is about 100 pg to about 10,000 ng, or about 100 pg to about 5,000 ng, or about 100 pg to about 1,000 ng. In some embodiments, the amount of peptide, biomarker, or plurality of Nfl isoforms is about 500 pg to about 10,000 ng, or about 500 pg to about 5,000 ng, or about 500 pg to about 1,000 ng. In some embodiments, the amount of peptide, biomarker, or plurality of Nfl isoforms is about 1,000 pg to about 10,000 ng, or about 1,000 pg to about 5,000 ng, or about 1,000 pg to about 1,000 ng. In some embodiments, the amount of peptide, biomarker, or plurality of Nfl isoforms is about 5 ng to about 10,000 ng, or about 5 ng to about 5,000 ng, or about 5 ng to about 1,000 ng. In some embodiments, the amount of peptide, biomarker, or plurality of Nfl isoforms is about 10 ng to about 10,000 ng, or about 10 ng to about 5,000 ng, or about 10 ng to about 1,000 ng.

In each of the above embodiments, the compositions may further comprise one or more anti-Nfl epitope-binding agents as described in section II and incorporated into this section by reference.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention. Those of skill in the art should, however, in light of the present disclosure, appreciate that many changes can be made in the specific embodiments that are disclosed and still obtain a like or similar result without departing from the spirit and scope of the invention, therefore all matter set forth or shown in the accompanying examples and drawings is to be interpreted as illustrative and not in a limiting sense.

### Example 1

This example describes an immunization protocol for production of NfL monoclonal antibodies.

Antigen: recombinant protein (neurofilament light chain-human, produced in E. coli. and purified). NfL 1-543aa was produced and purified. One hundred micrograms of recombinant NfL protein was injected intraperitoneally (IP) into B6/C3 mice. The first injection of consisted of 100 micrograms of recombinant NfL 1-543aa per mouse in 200 microliters of μl of PBS+200 microliters of complete Freund's adjuvant IP. A second injection 14 days later consisted of 100 micrograms of recombinant NfL 1-543aa in 200 microliters of microliters of PBS plus 200 microliters of incomplete Freund's adjuvant IP per mouse. After 21 days, thirty microliters of blood were removed from each mouse and serum was screened by a direct ELISA against the recombinant NfL. Mice received a third injection after 28 days of 100 micrograms of recombinant NfL 1-543aa in 200 μl PBS plus 200 microliters of incomplete Freund's adjuvant IP. Thirty microliters of blood were removed from mice after 35 days (second bleed) and serum was again assessed by direct ELISA against the recombinant NfL. If the titers of a mouse were over 1:10,000, fusion then occurred. A final boost of 50 micrograms of recombinant NFL1-543aa in PBS was then injected IP to mice 3 days before fusion.

Antibodies being produced from individual clones were then screened by coating plates by with recombinant NFL 1-543aa. To do this, NfL 1-543aa was to plates, and the plates were then blocked the plates with 1% milk. The plates were then washed with PBS and cell culture media was added from the individual clones at a dilution of 1:10 in 0.5% BSA in PBS buffer plus anti-mouse IgG HRP. TMB was then added nd read by the absorbance at 650 and compared this to cell culture media alone as a blank.

Twenty-three antibodies were selected for further characterization (see Example 2). Antibodies HJ30.1, HJ30.2, HJ30.4, HJ30.7, HJ30.11, and HJ30.13 were deposited with the ATCC.

TABLE 1
Antibody
(ATCC # of the hybridoma)

HJ30.1 (PTA-126966)
HJ30.2 (PTA-126967)
HJ30.4 (PTA-126968)
HJ30.7 (PTA-126969)
HJ30.11 (PTA-126970)
HJ30.13 (PTA-126971)

### Example 2

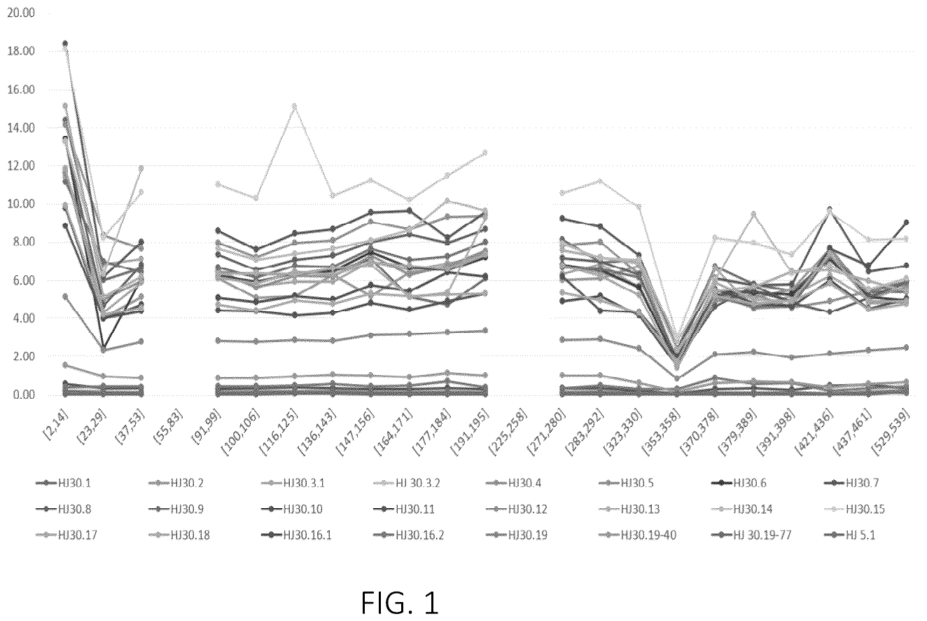

In this example, Nfl was immunoprecipitated using one of the twenty-three antibodies described in Example 1, proteolytically digested, and then proteolytic peptides of Nfl were detected and quantified by mass spectrometry (MS). In all MS experiments described in this and other examples, when a reliable MS signal was not detected for a given tryptic peptide of the ISTD, no data was reported for either the ISTD (15N) or experimental Nfl (14N)—see, for example, the tryptic peptide [55,83] in FIG. 1. Tryptic peptides are identified throughout the specification, including in the examples herein, using a particular shorthand notation—“[X, Y]”—wherein X is the first amino acid of the tryptic peptide and Y is the last amino acid of the tryptic peptide, and wherein X+1 or Y+1 identifies the amino acid of full-length Nfl (SEQ ID NO: 1). For instance, the nomenclature “[55,83]” is shorthand for the tryptic peptide SYSSSSGSLMPSLENLDLSQVAAISNDLK (SEQ ID NO: 5), which consists of amino acids 56 to 84 of full-length Nfl (SEQ ID NO: 1).

To facilitate recovery of immunoprecipitated Nfl, individual antibodies were cross-linked to Dynabeads® at a concentration of 25 μg antibody per mg of beads, per manufacturer instructions. HJ5.1, an anti-Aβ antibody, was used as a negative control. Full-length, recombinant Nfl (rec-Nfl) and biologically produced Nfl isolated from brain lysate or CSF were evaluated.

For full-length, recombinant Nfl analysis (rec-Nfl), 10 uL of 5 ng/uL rec-Nfl in 1% HSA were added to 40 uL of 100 mM TEABC. Nfl was immunoprecipitated by adding 30 uL of a 30% (i.e., 3 mg/mL) slurry of an antibody-conjugated bead preparation, and rotating the sample for 120 minutes at room temperature. The antibody-conjugated beads were magnetically separated, and the post-IP supernatant was removed. The beads were washed three times in 1 mL of 25 mM TEABC (per wash).

NfL was also affinity purified from brain lysate and CSF. The brain samples used were previously lysed samples, stored at −80° C. for assay development.

Patient demographics for the subjects were not obtained (e.g., age, sex, clinical signs or symptoms of neuronal disease). Frozen lysates were thawed and an aliquot diluted 1:1000 with 1% HSA. 450 μl of the thawed brain lysate was transferred to a 1.6 mL new tube. 25 uL of a master mix containing detergent (1% NP-40), Chaotropic reagent (5 mM Guanidine), and protease inhibitors (Roche complete Protease Inhibitor Cocktail), and 20 uL of 0.5 ng/ml Nfl internal standard (ISTD) in 50 mM triethyl ammonium bicarbonate buffer (TEABC) was then added. Lys, Arg, 13C and 15N labeled recombinant, full-length Nfl was used as the ISTD. Nfl was immunoprecipitated by adding 30 uL of a 30% (i.e., 3 mg/mL) slurry of an antibody-conjugated bead preparation, and rotating the sample for 120 minutes at room temperature. The antibody-conjugated beads were magnetically separated, and the post-IP supernatant was removed. The beads were washed three times in 1 mL of 25 mM TEABC (per wash).

The CSF samples used were previously obtained from human subjects for assay development and stored at −80° C. Patient demographics for the subjects is not known (e.g., age, sex, clinical signs or symptoms of neuronal disease). Frozen CSF samples were thawed at room temperature, and 500 μl of the thawed CSF was transferred to a new tube. 25 μL of a master mix containing detergent (1% NP-40), Chaotropic reagent (5 mM Guanidine), and protease inhibitors (Roche complete Protease Inhibitor Cocktail), and 20 μL of 0.5 ng/ml Nfl internal standard (ISTD) in 50 mM triethyl ammonium bicarbonate buffer (TEABC) was then added. Lys, Arg, 13C and 15N labeled recombinant, full-length Nfl was used as the ISTD. Nfl was immunoprecipitated by adding 30 ul of a 30% (i.e., 3 mg/mL) slurry of an antibody-conjugated bead preparation, and rotating the sample for 120 minutes at room temperature. The antibody-conjugated beads were magnetically separated, and the post-IP supernatant was removed. The beads were washed three times in 1 mL of 25 mM TEABC (per wash).

Regardless of the sample, antibody-bound Nfl was digested on-beads with 400 ng MS-grade trypsin or Lys-C in 25 mM TEABC for 16 hours at 37° C. Concentration of protease, incubation time, and buffer can be adjusted according to methods known in the art. Digests were loaded onto TopTip C18 conditioned per manufacturer instructions, desalted by washing twice with 0.1% formic acid (FA), and eluted with 60% acetonitrile (ACN) and 0.1% FA. The eluted peptides were dried by vacuum centrifugation and either stored at −80° C. or resuspended immediately.

For the MS analysis, eluted peptides were reconstituted with 25 uL of 0.1% FA/0% CAN. A 4.5 uL aliquot of each digest was then injected into nano-Acquity LC for MS analysis. The nano-Acquity LC (Waters Corporation, Milford, MA, USA) was fitted with HSS T3 75 μm×100 μm, 1.8 μm column and a flow rate of 0.5 μL/min. Peptides were separated over a gradient from 2% solvent B to 95% solvent B. Solution A was composed of 0.1% formic acid in MS grade water and solution B was composed of 0.1% formic acid in acetonitrile. Samples were analyzed in positive ion mode, with a spray voltage of 2,200V and ion transfer tube temperature of 275° C. Data were collected with parallel reaction monitoring (PRM) for endogenous (N14) and isotopically labeled (C13N15) peptides, targeting the 2+or 3+charge state. HCD Collision energy ranged from 18-27% and was optimized for each peptide. Maximum injection time ranged from 24 to 118 ms and was optimized for each peptide. Normalized AGC Target was set to 400%. Data were extracted using Skyline software (McCoss laboratory) and analyzed in Skyline and Microsoft Excel.

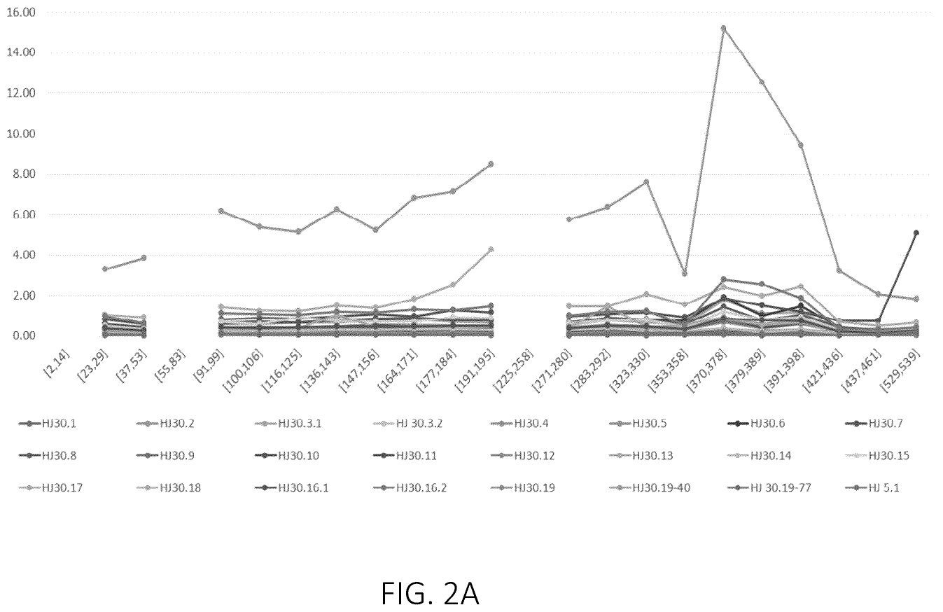

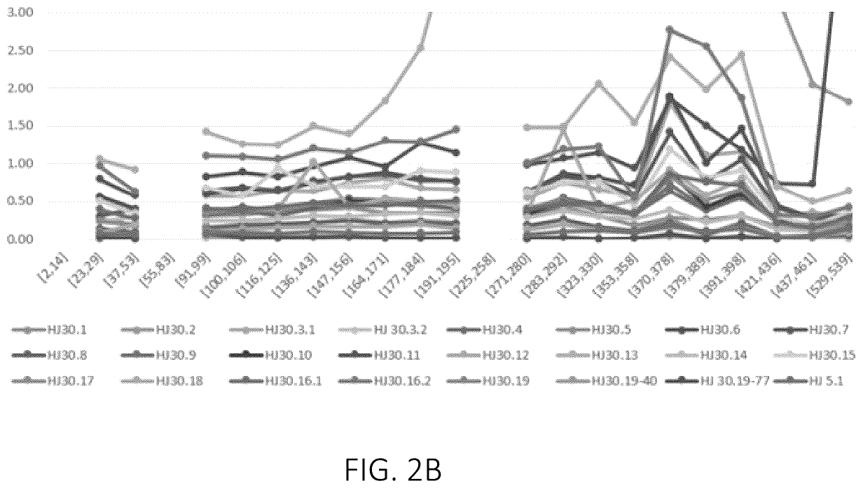

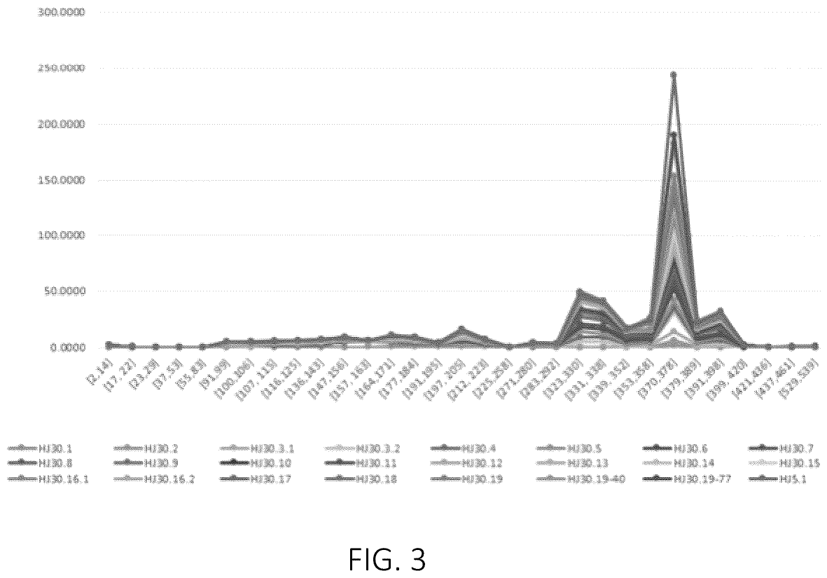

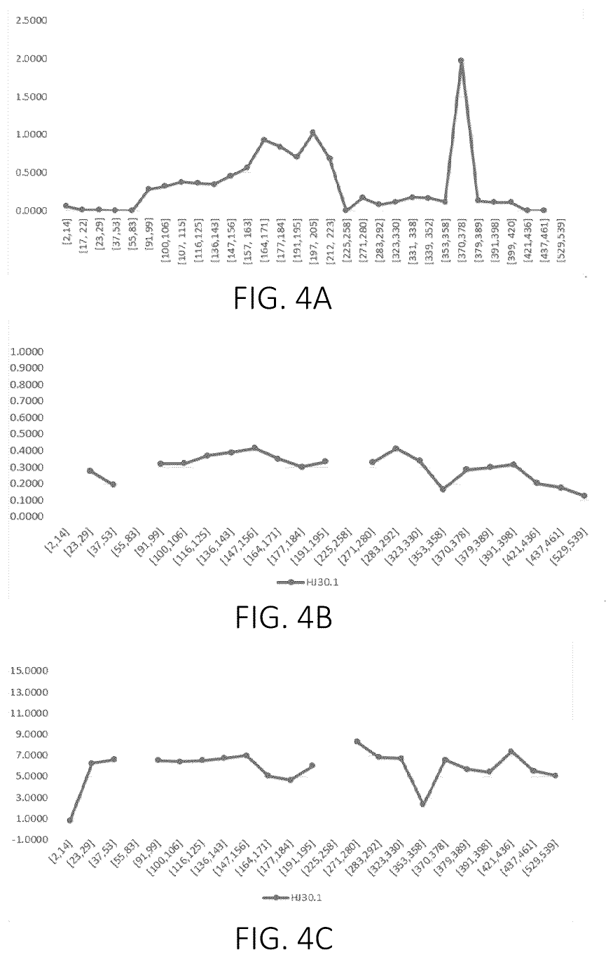

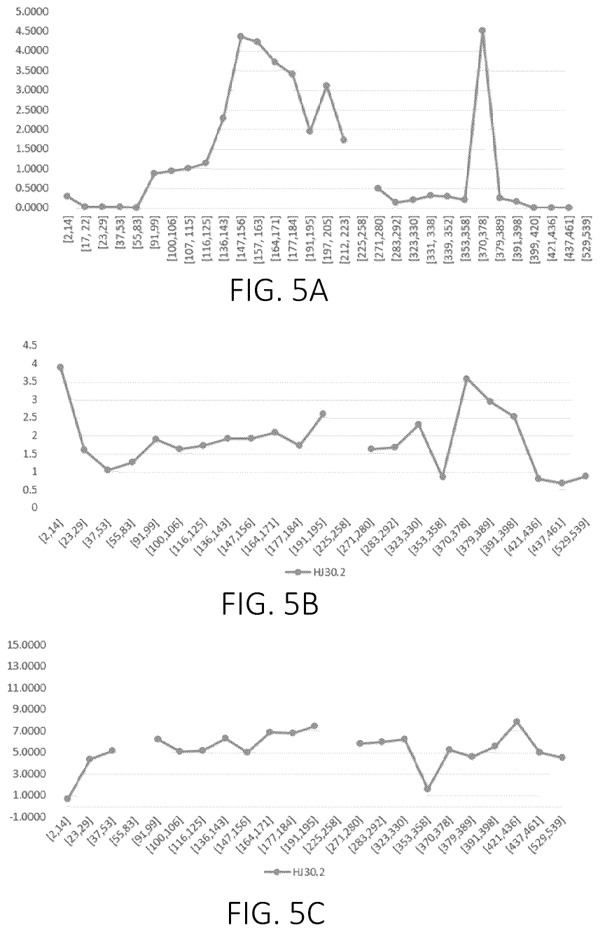

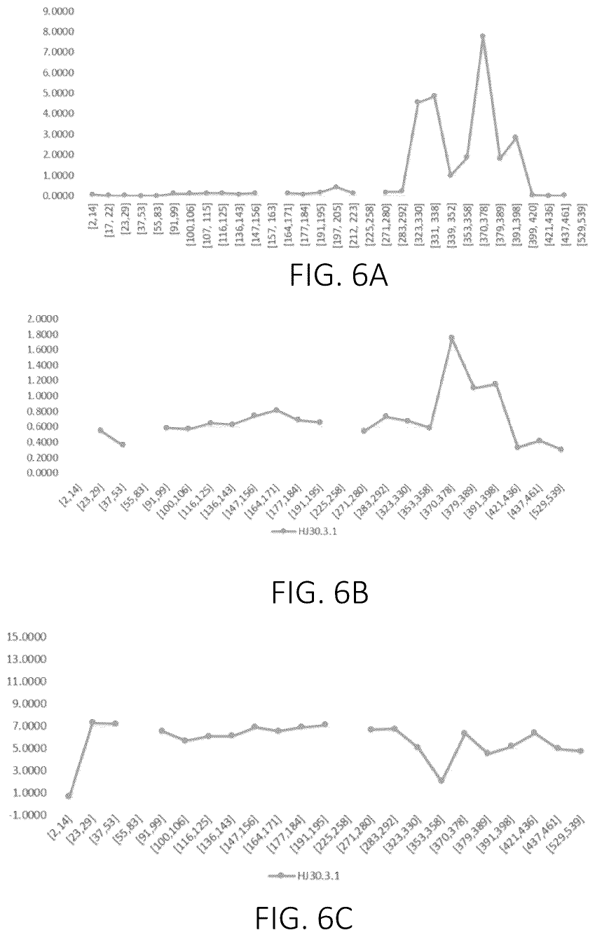

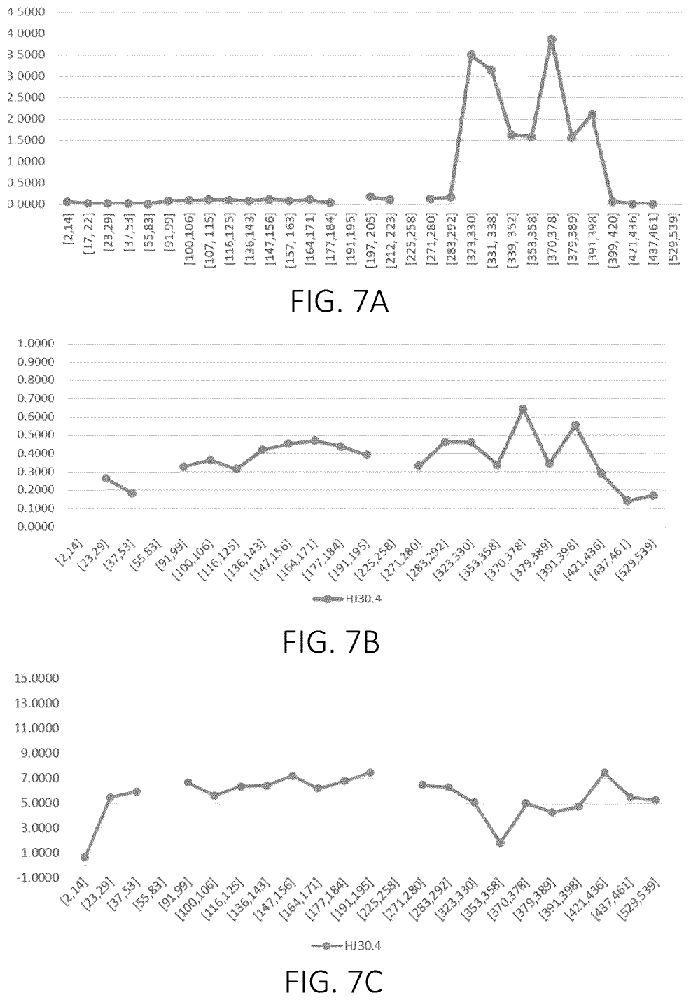

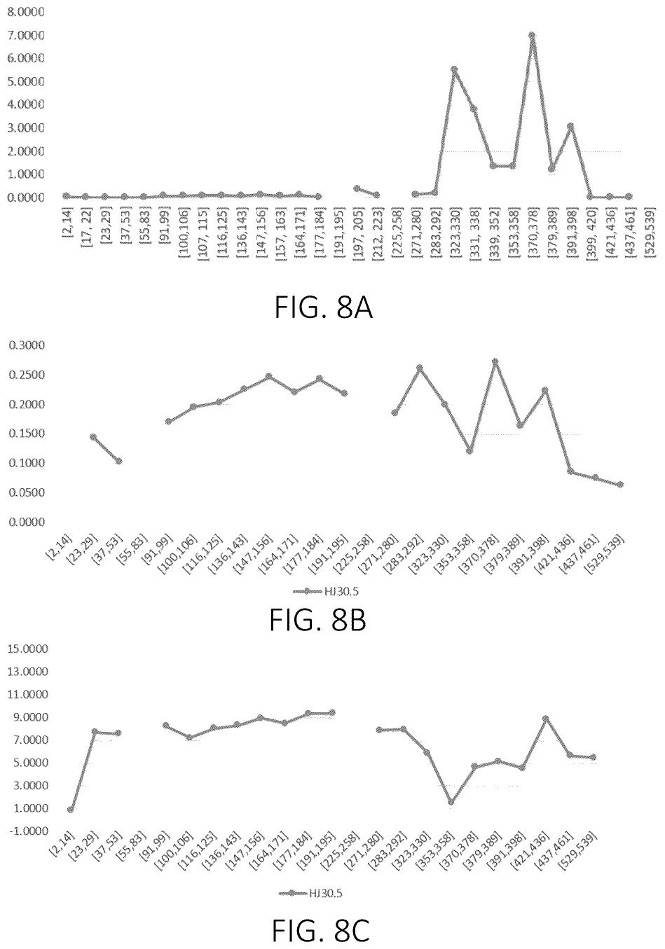

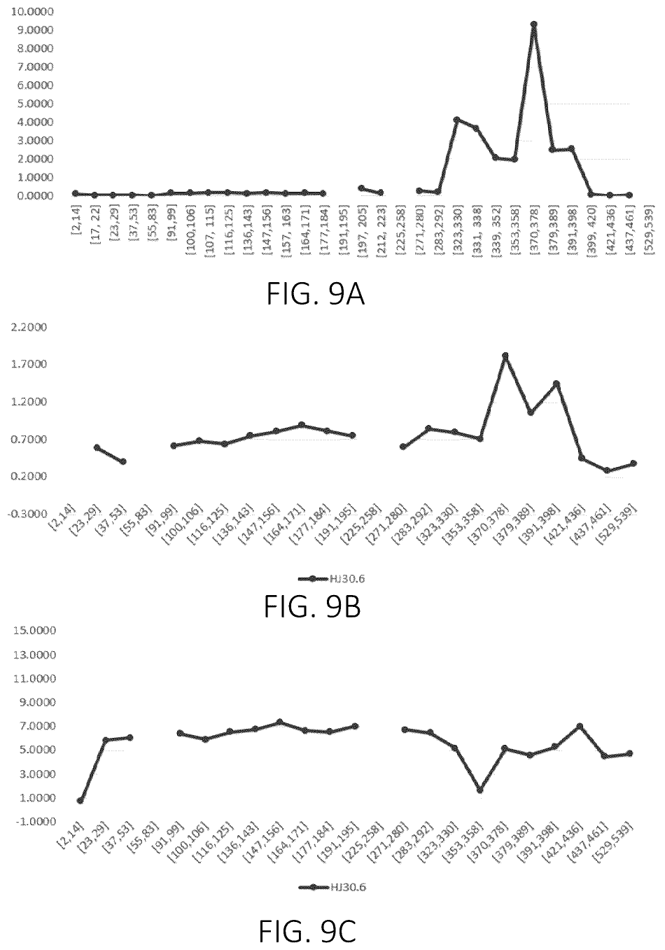

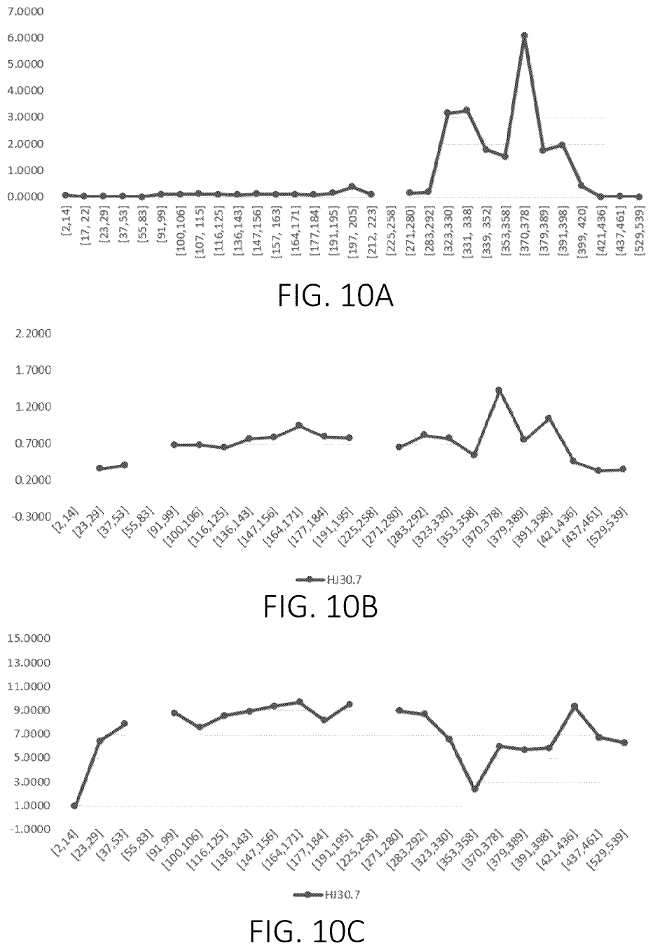

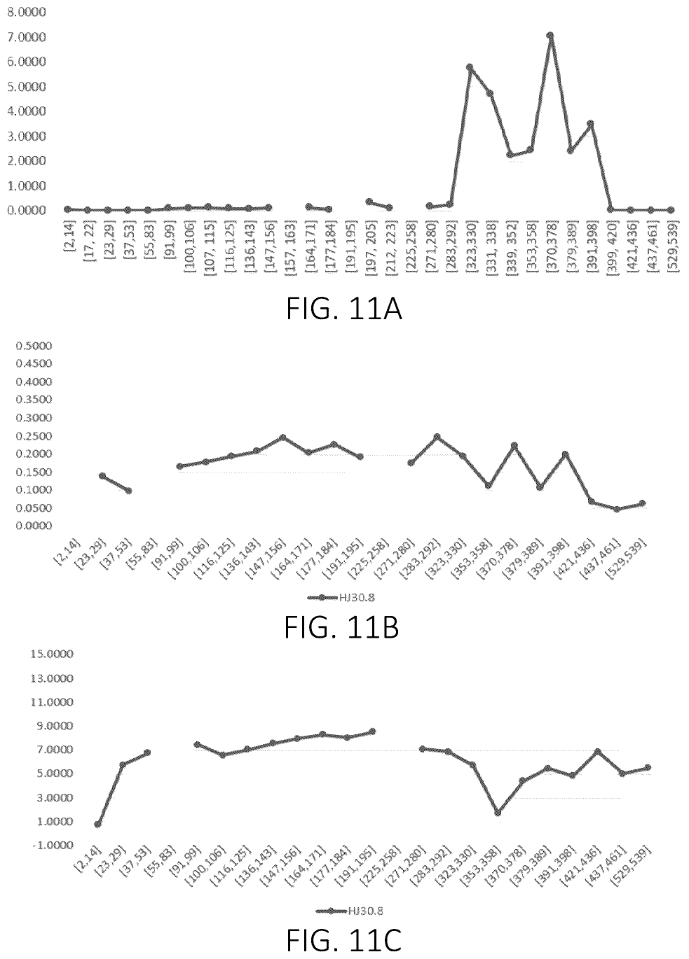

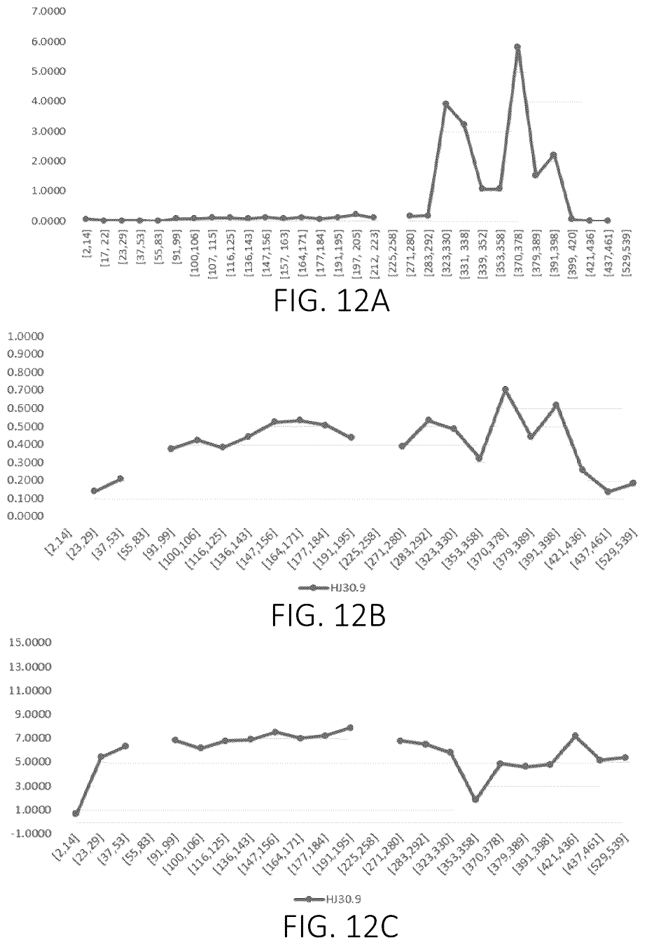

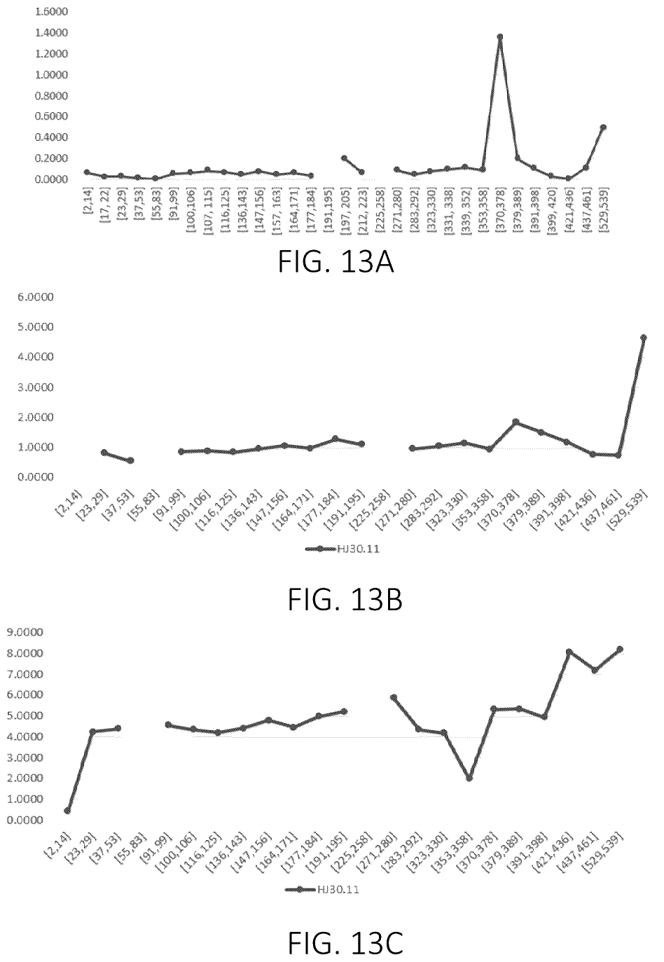

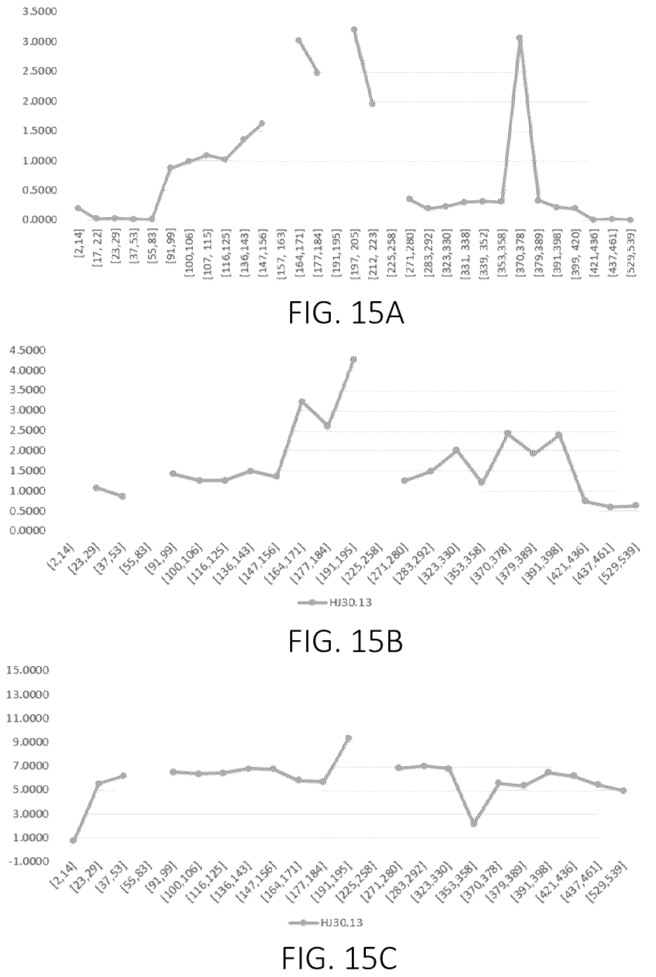

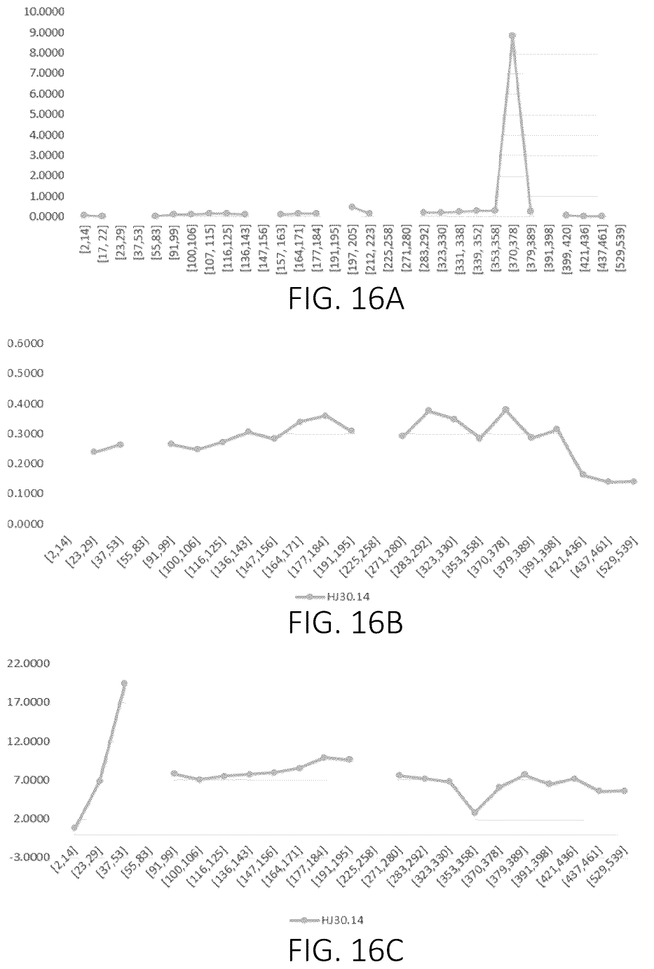

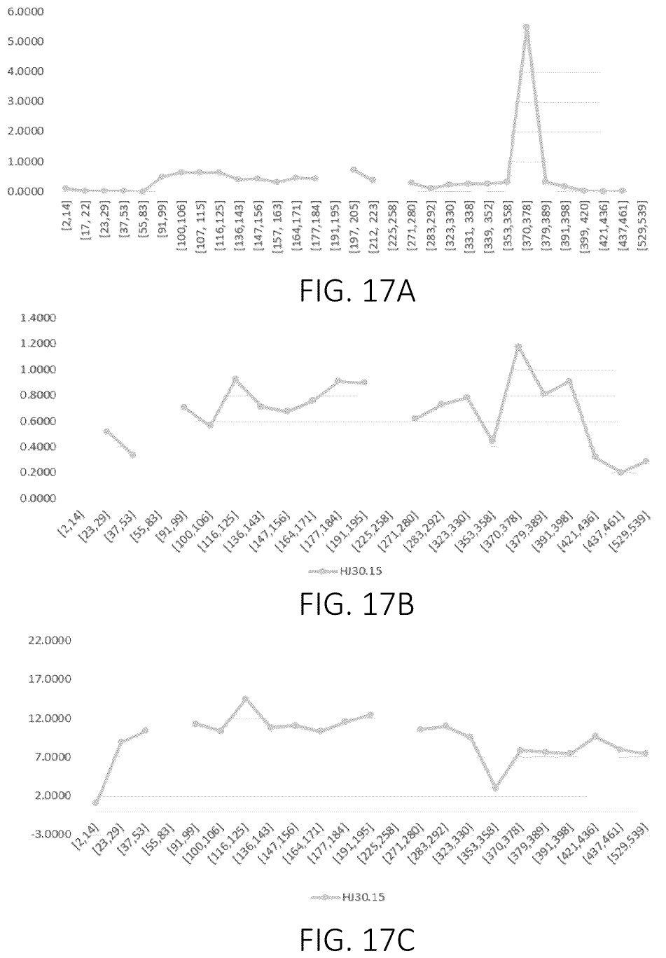

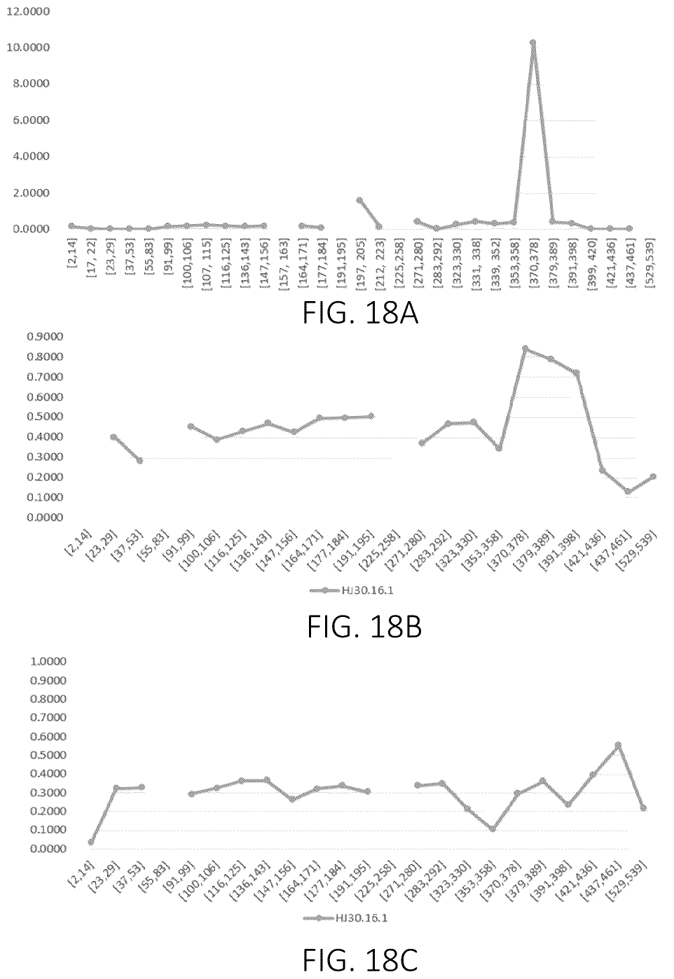

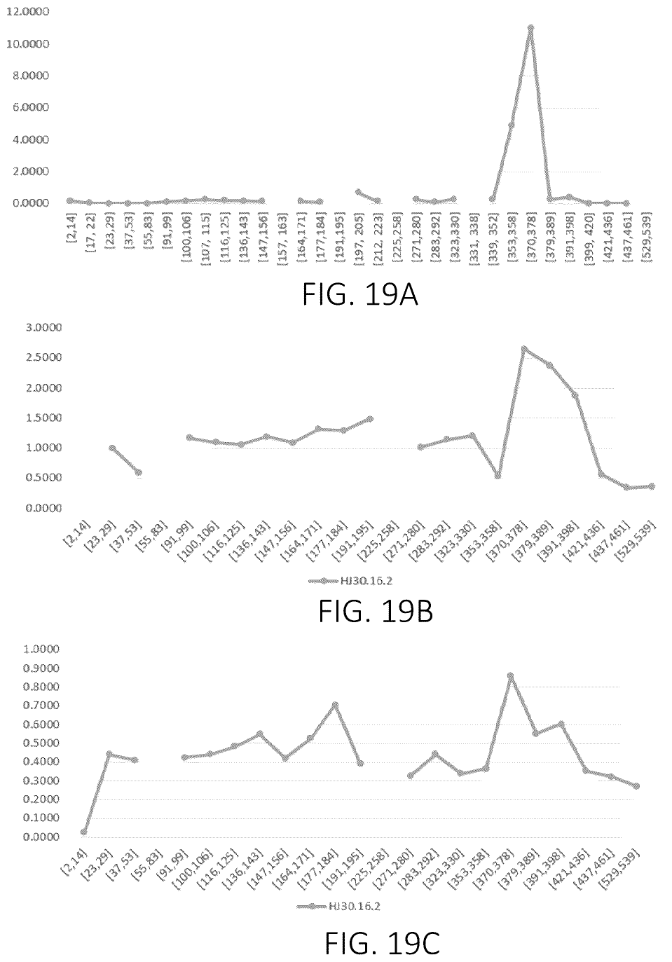

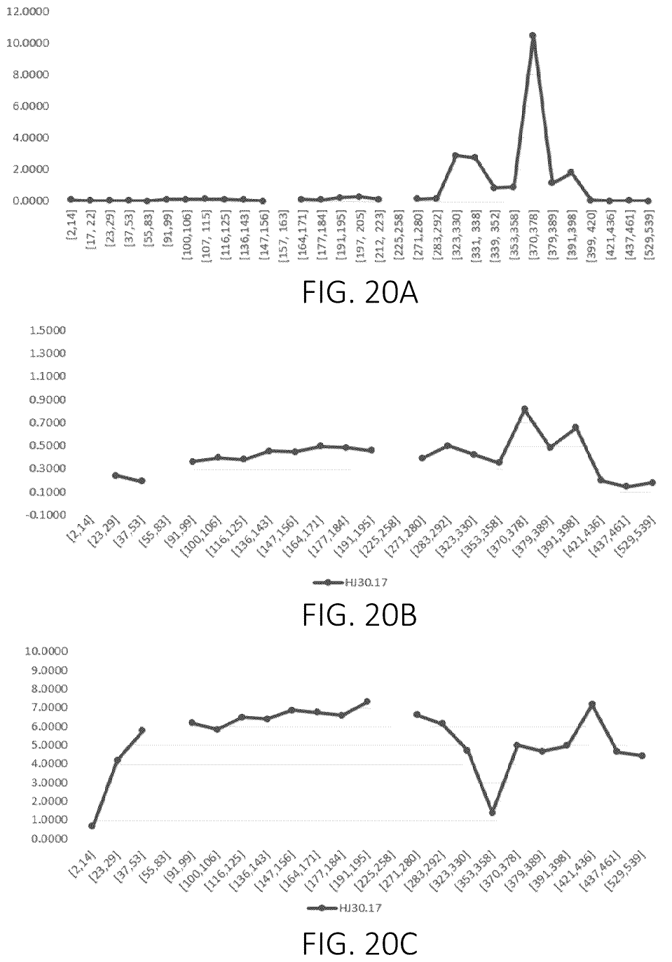

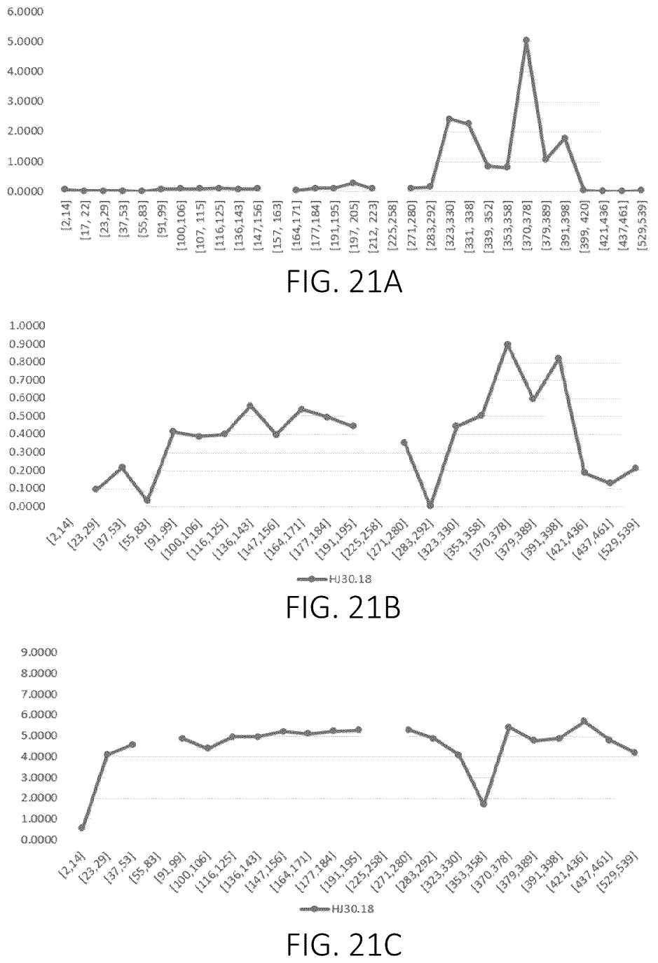

FIG. 1 provides an overview of Nfl recovery/IP efficiency for rec-Nfl. FIG. 2 provides an overview of Nfl recovery/IP efficiency from brain lysate. FIG. 3 provides an overview of Nfl recovery/IP efficiency from CSF. FIG. 4-21 show the data for individual antibodies. For each graph, the y-axis is 14N/15N ratio and each point on the x-axis is a tryptic peptide of Nfl.

In FIG. 1, 14N/15N ratios are consistent along the sequence, which was expected given that both the ISTD and the experimental Nfl (rec-Nfl) were full length proteins. Notably, HJ13.3.2, HJ30.19, HJ30.19-40, and HJ30-. 19-77 showed no rec-Nfl recovery.

In brain lysate, Nfl detected was also mostly full length (FIG. 2). Analysis indicated the N-terminal methionine was cleaved and the second amino acid (serine) is acetylated (data not shown). The high signal seen with tryptic peptides [370,378], [379,389], and [391,398] was determined to be due to a contaminant (data not shown).

The contamination seen in the analysis of the brain lysate samples was also seen in the analysis of the CSF sample (FIG. 3-21). Since the tryptic peptides [370,378], [379,389], and [391,398] are not unique to Nfl, they should not be used for detection and quantification of Nfl in a biological sample. Acetylation of [2,14] was also seen in CSF Nfl (FIG. 3-21). Unlike the 14N/15N ratios seen with rec-Nfl and Nfl from brain lysate, Nfl from CSF showed inconsistent 14N/15N ratios along the sequence, indicating a number of truncated Nfl species in CSF (FIG. 3). Comparing the profiles of individual antibodies (FIG. 4-21), it was evident that certain antibodies are more effective at immunoprecipitating CSF Nfl than others, and that CSF Nfl contains a plurality of Nfl isoforms of varying length with cleavage at the N-terminus, C-terminus, or N-terminus and C-terminus.

A summary of some of the potential isoforms enriched from brain lysate and CSF by the methods described above are illustrated in FIG. 22 with approximate sizes provided.

### Example 3

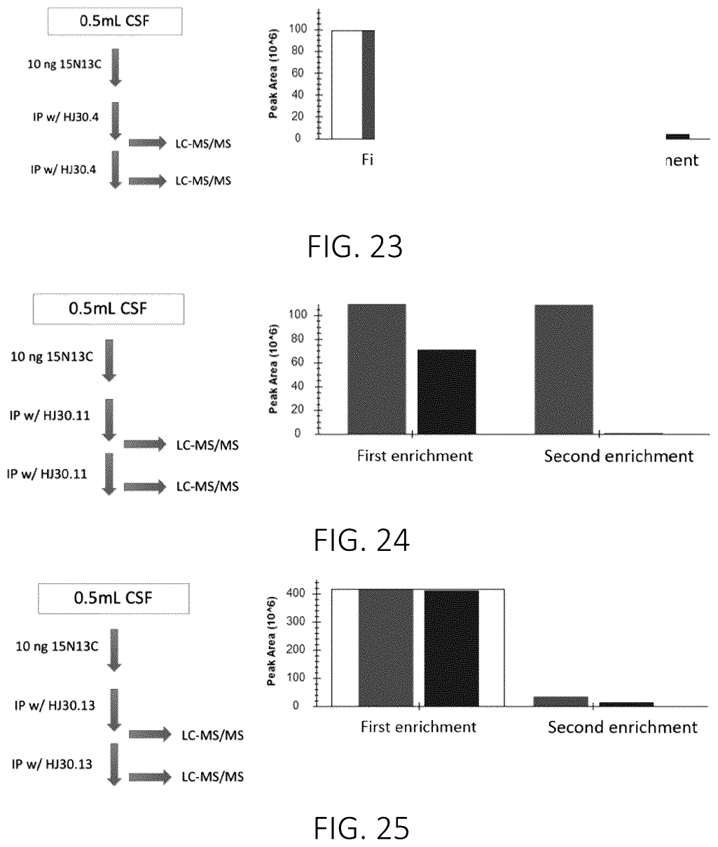

In this example, Nfl was immunoprecipitated from CSF using one of HJ30.4, HJ30.11, or HJ30.13. The CSF samples used were previously obtained from human subjects for assay development and stored at −80° C. Patient demographics for the subjects is not known (e.g., age, sex, clinical signs or symptoms of neuronal disease). Immunoprecipitation and LC-MS analysis was as generally described in Example 2, except the supernatant recovered after immunoprecipitation was subjected a second round of immunoprecipitation with the same antibody. The 14N (red bars) and 15N (blue bars) data are shown separately (FIGS. 23-25). The first two bars in each figure correspond to the first enrichment step and the second two bars correspond to the second enrichment step.

Most of the internal standard (ISTD, blue bar) is recovered from the sample by the first enrichment step. This suggests a full-length internal standard would need to be re-added if sequential rounds of enrichment with the same or different epitope-binding agent are performed. Similarly, HJ30.4 and H30.13 efficiently enriched Nfl isoforms from CSF in a single step (FIGS. 23-24). In contrast, HJ30.11 did not immunoprecipitate in a single step all the Nfl isoforms it specifically binds (FIG. 25).

### Example 4

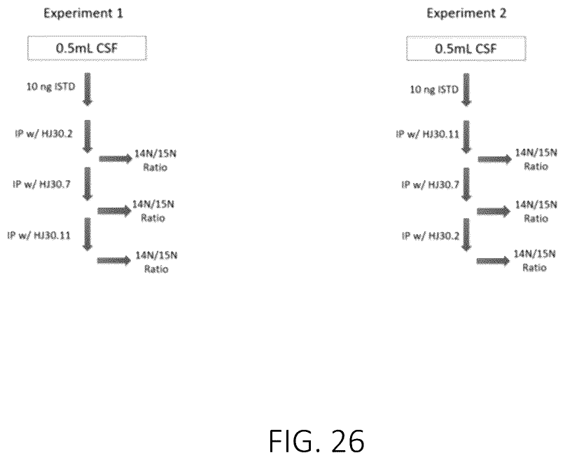

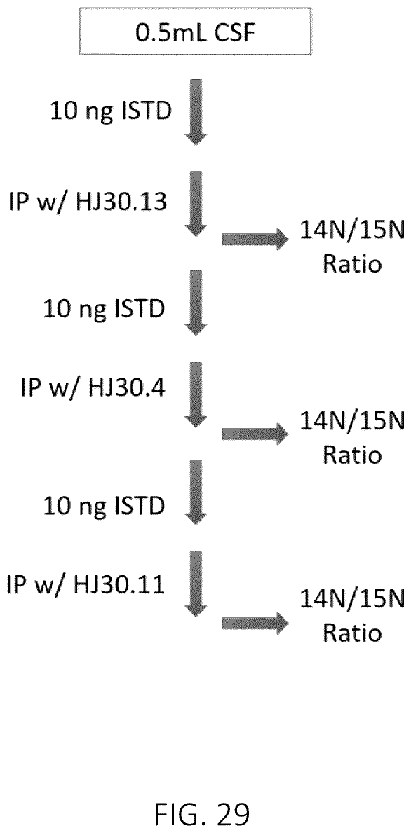

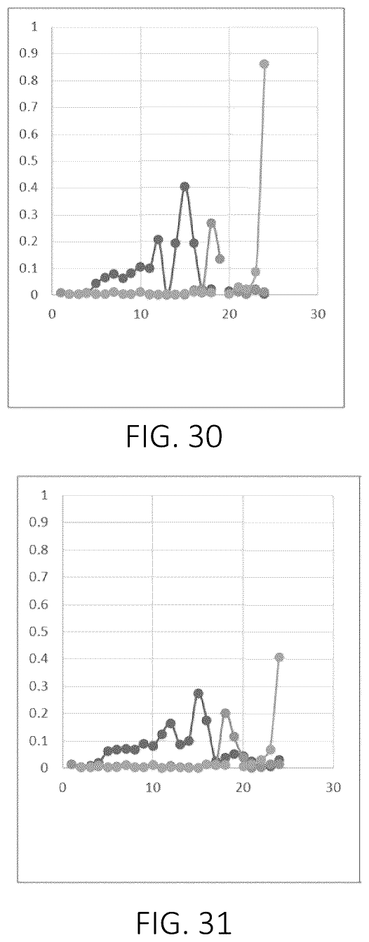

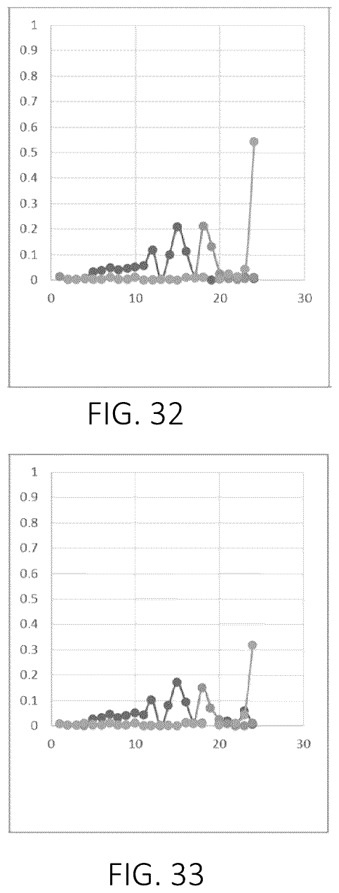

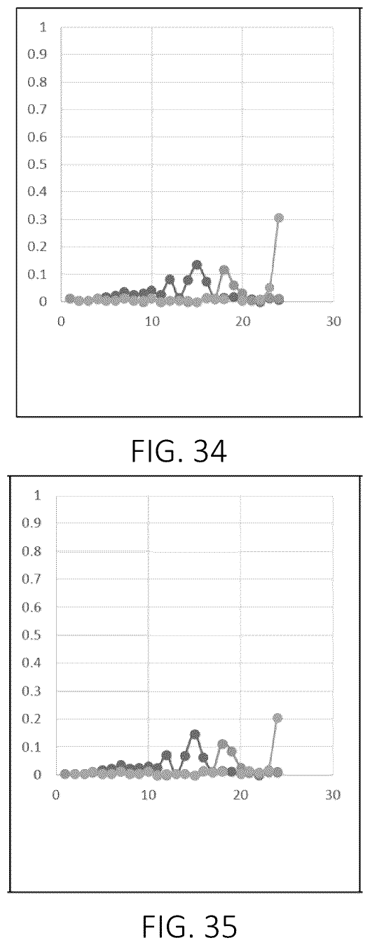

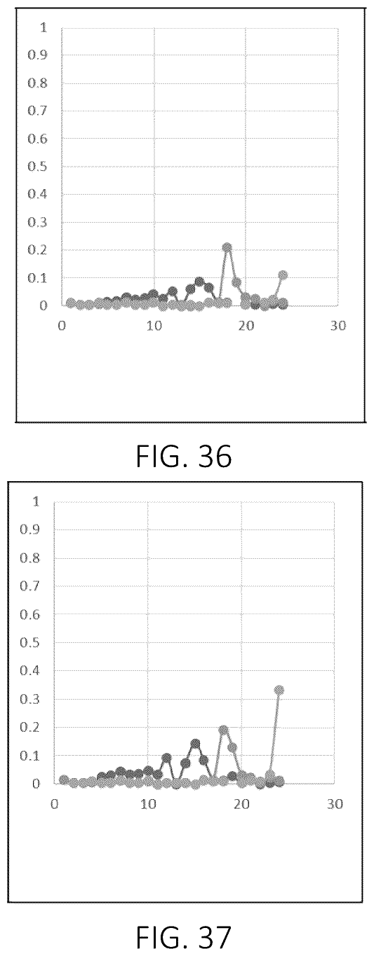

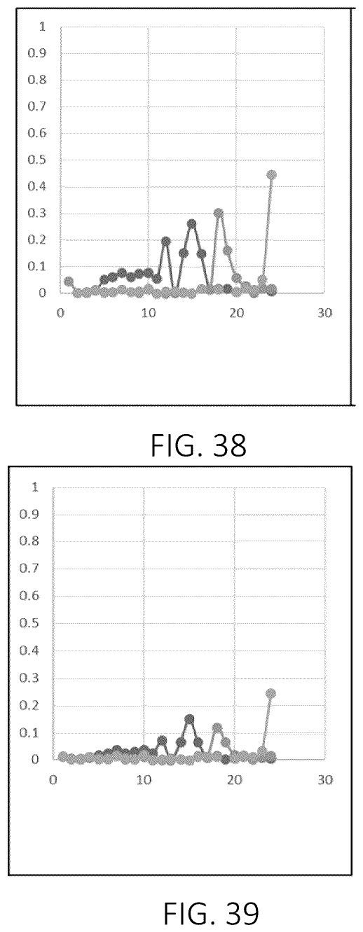

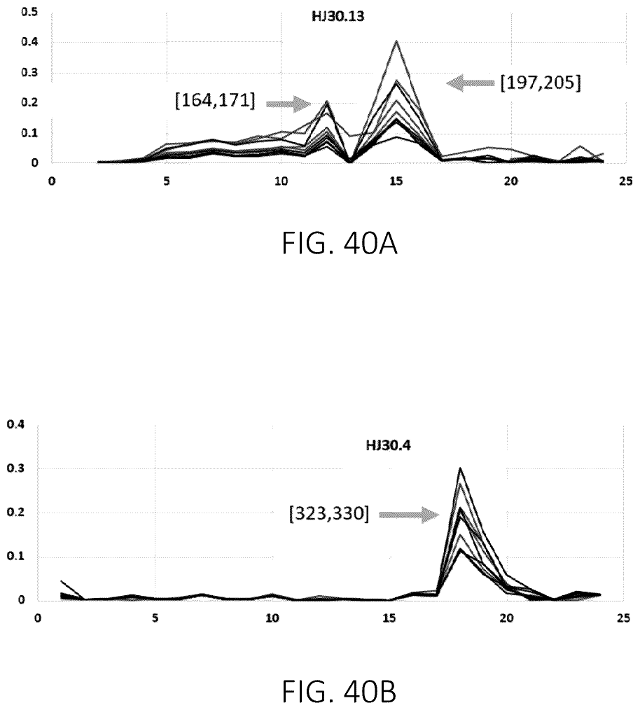

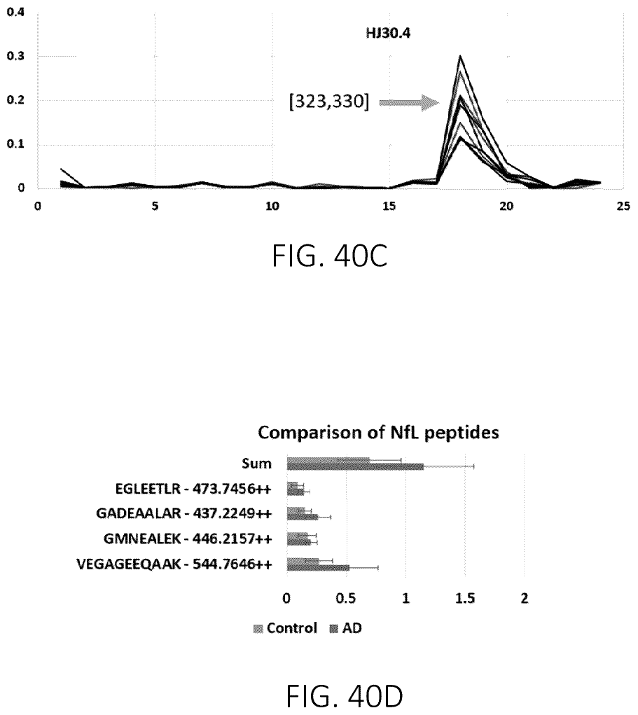

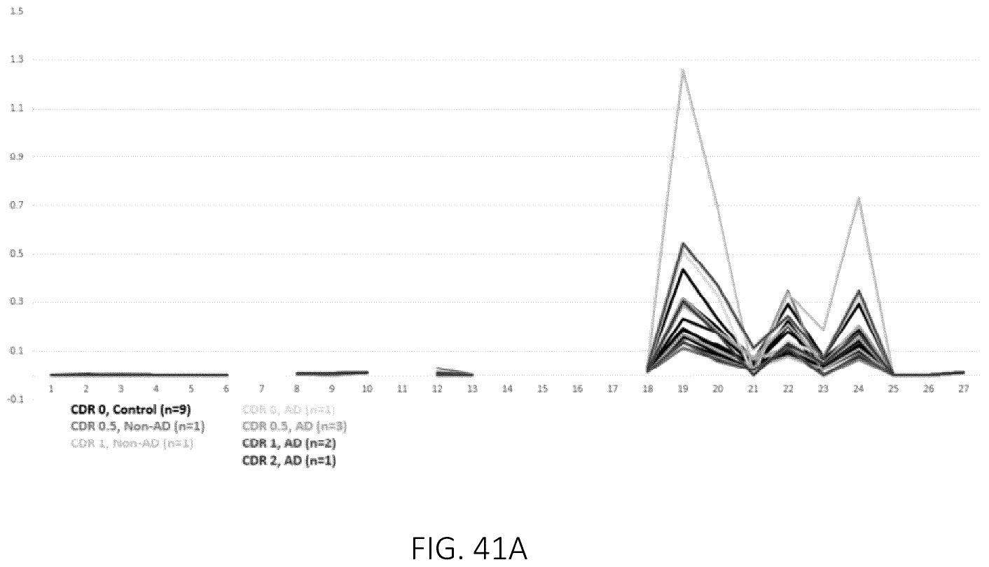

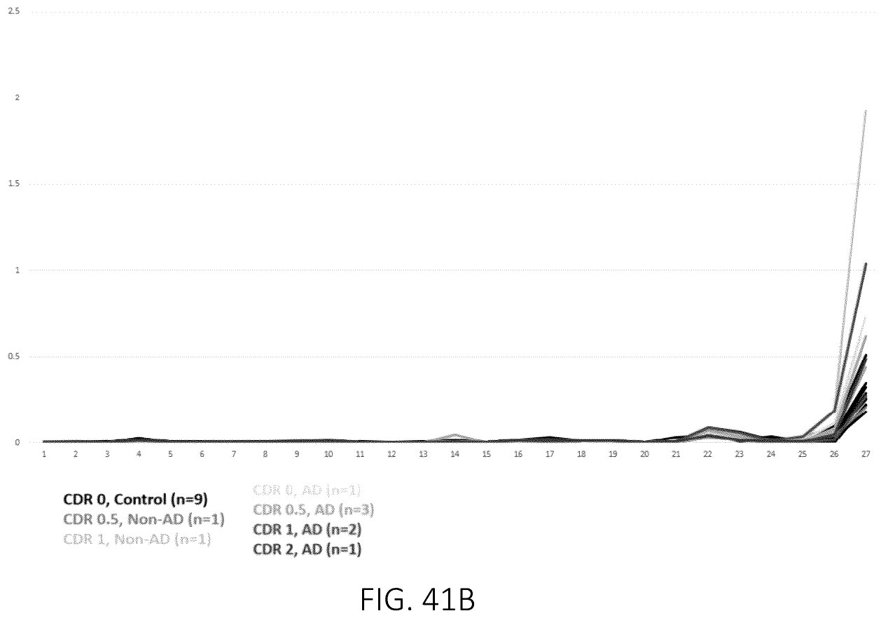

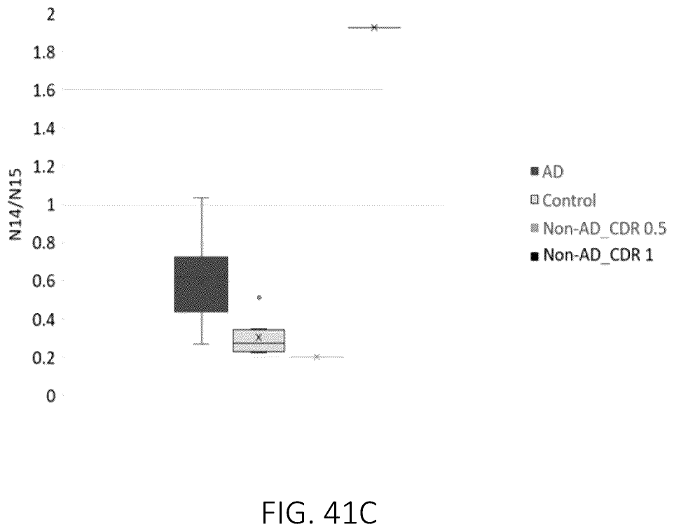

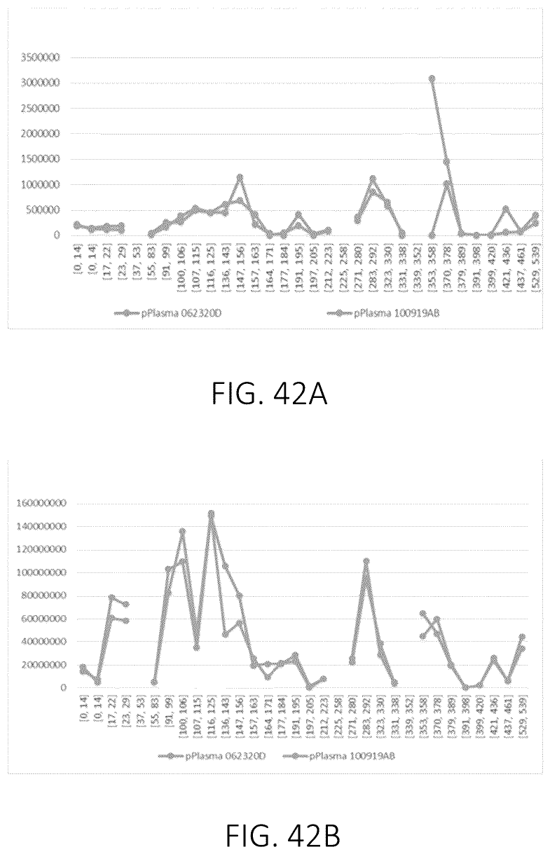

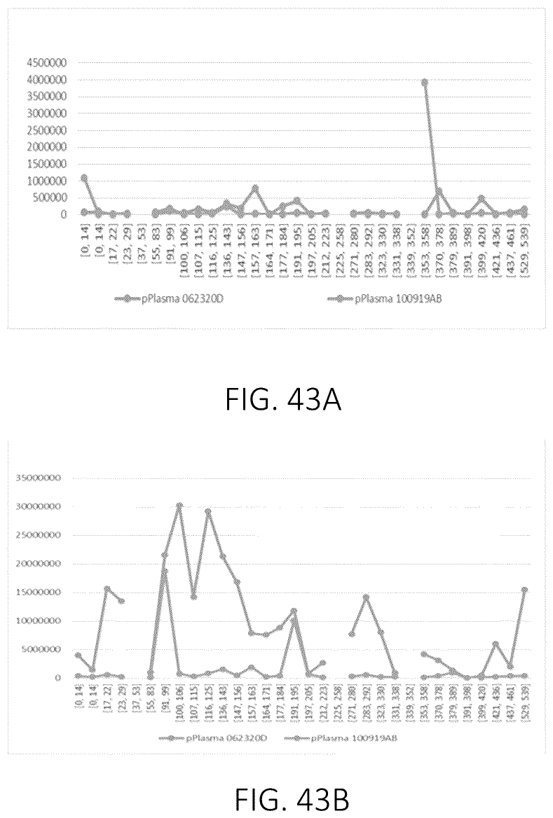

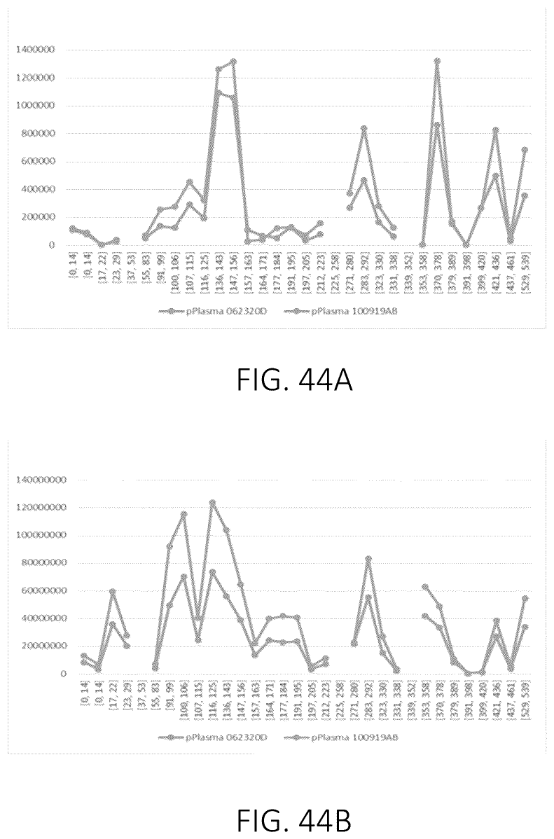

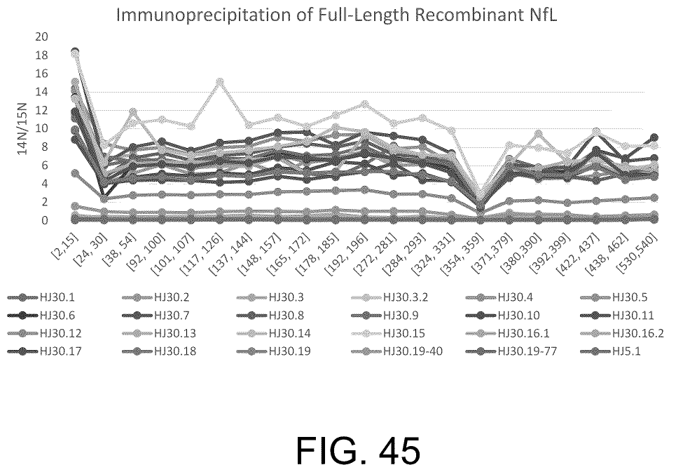

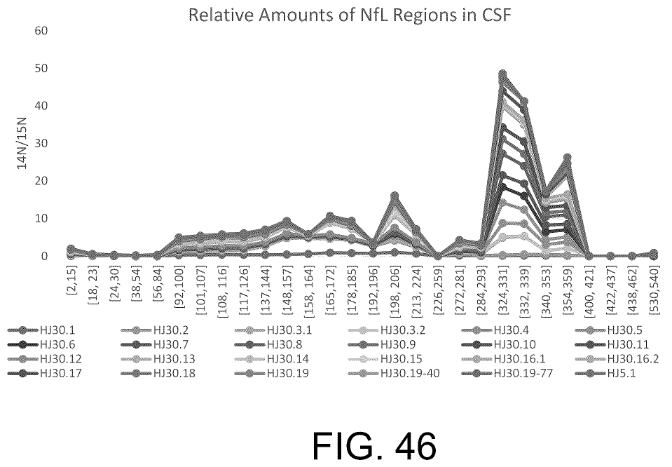

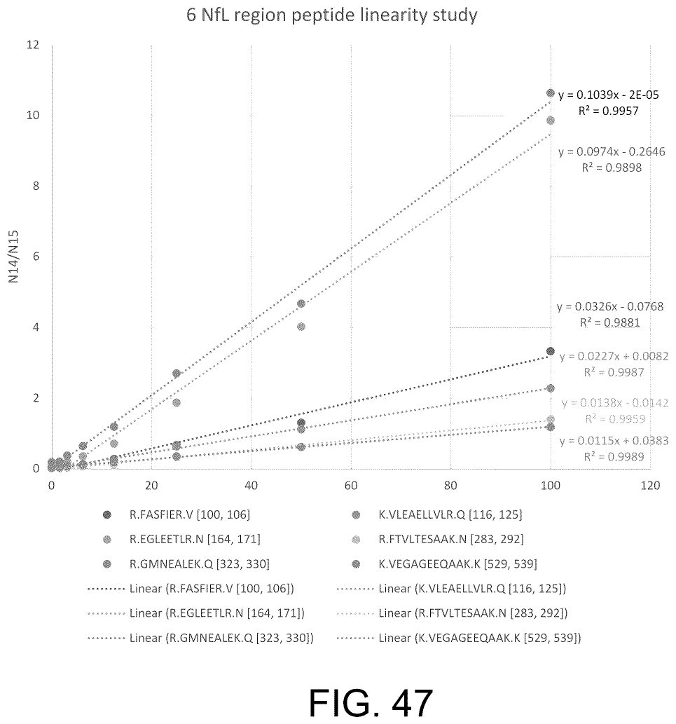

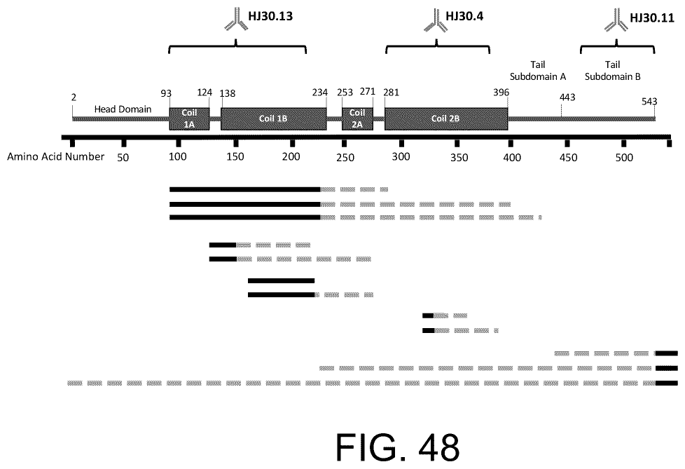

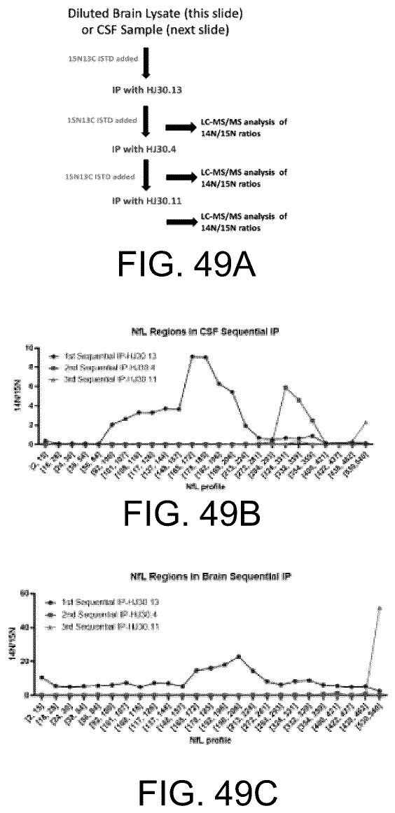

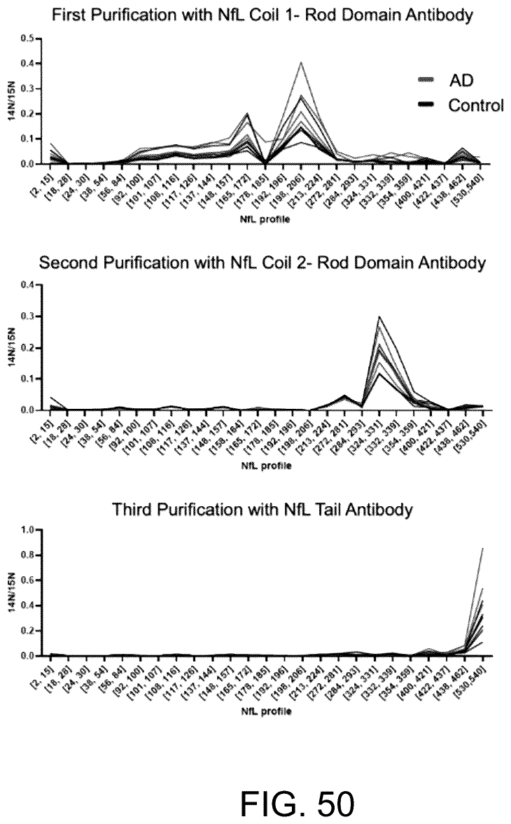

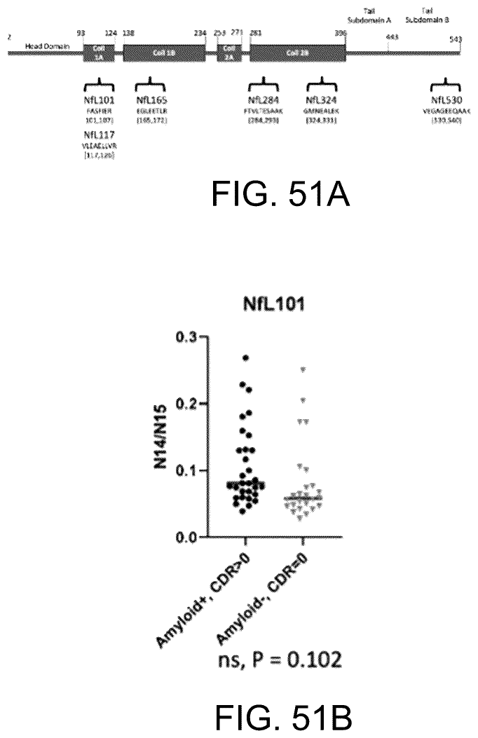

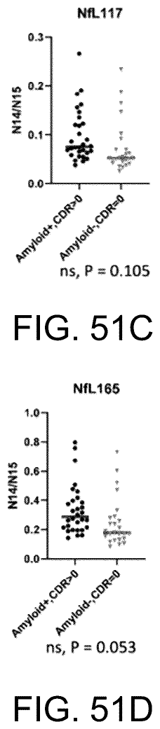

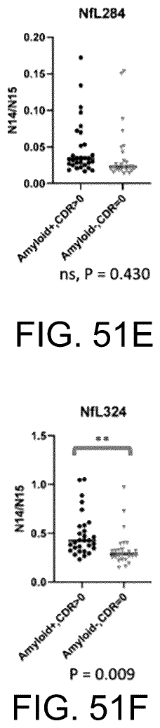

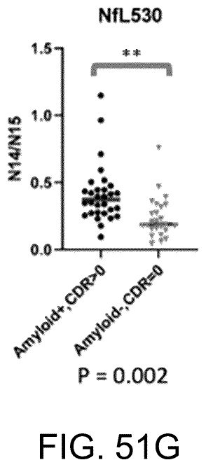

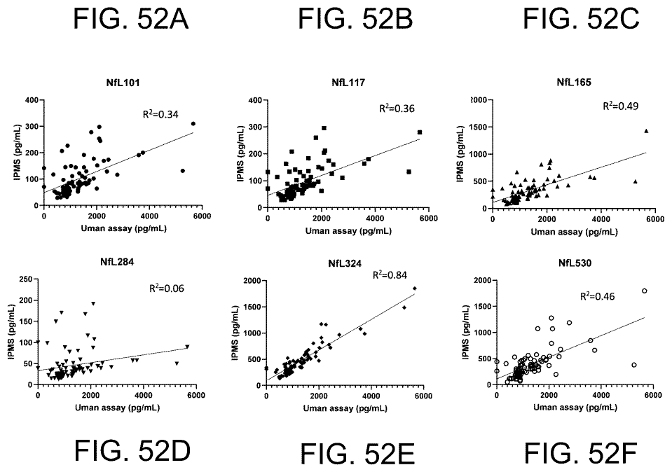

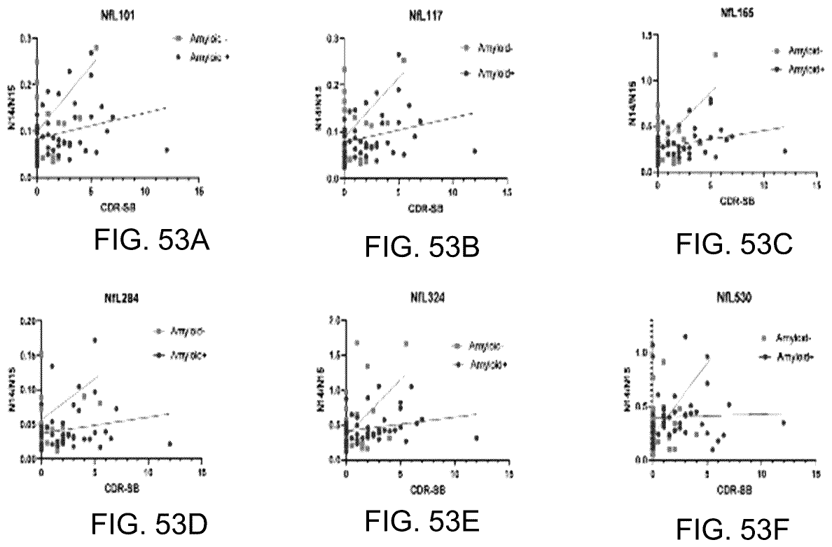

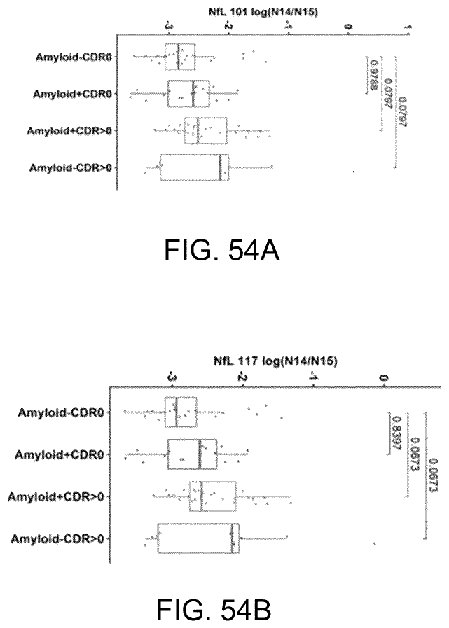

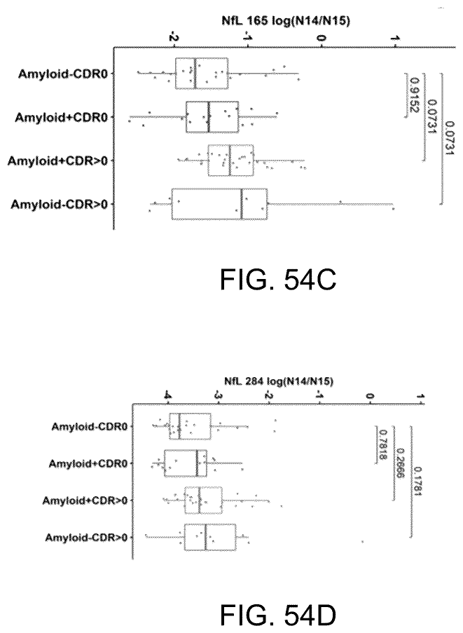

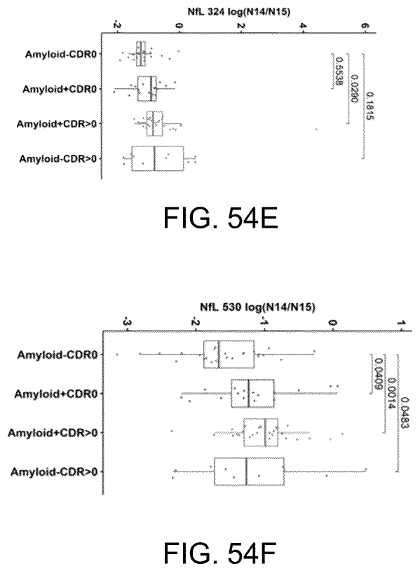

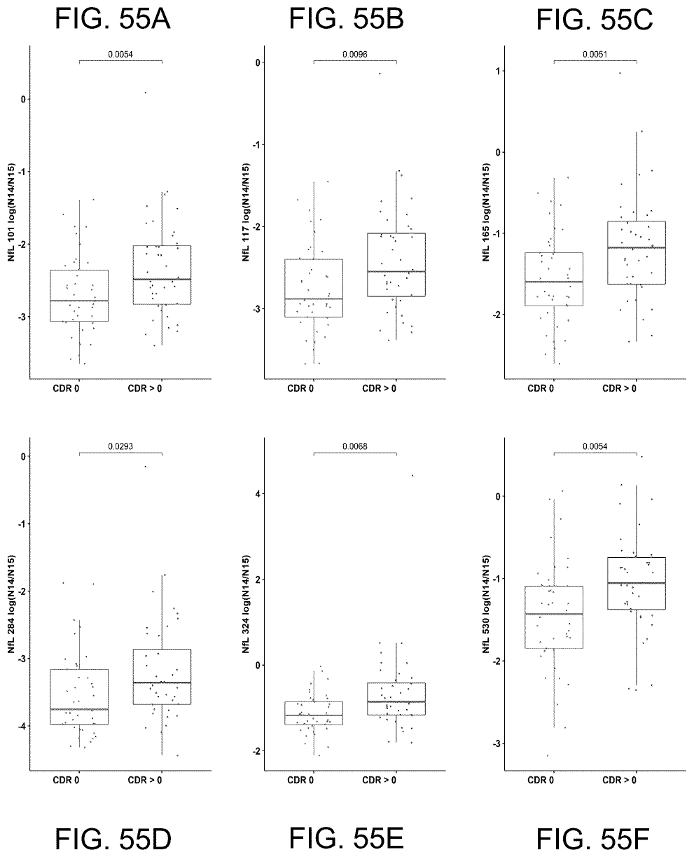

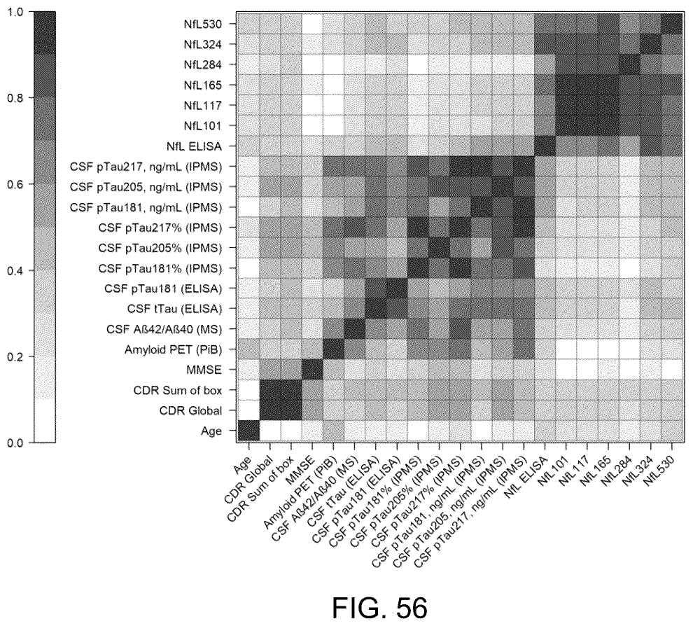

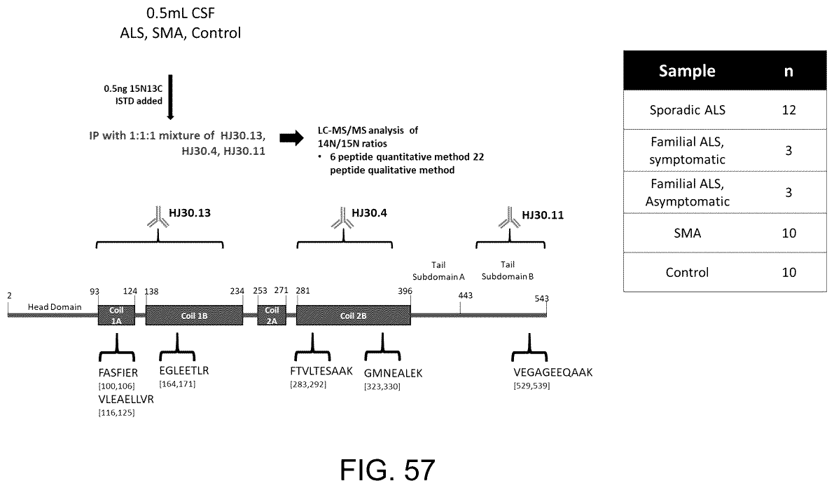

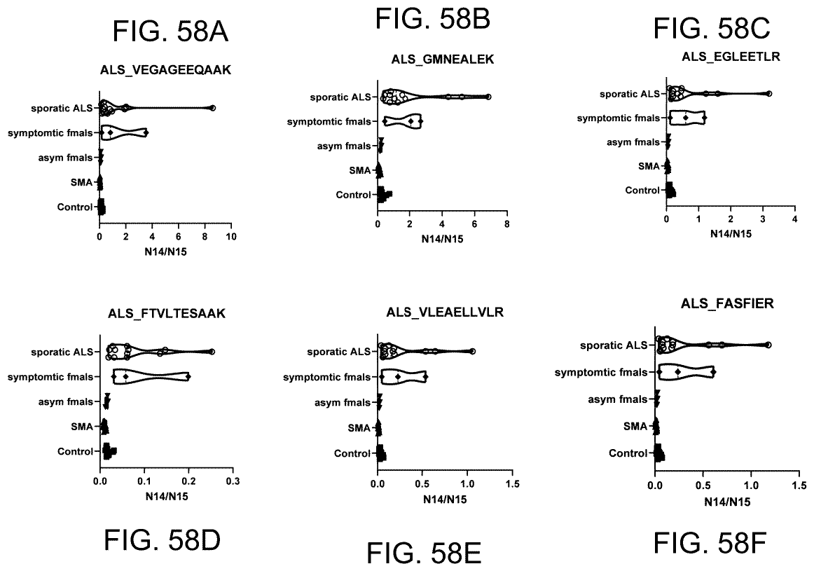

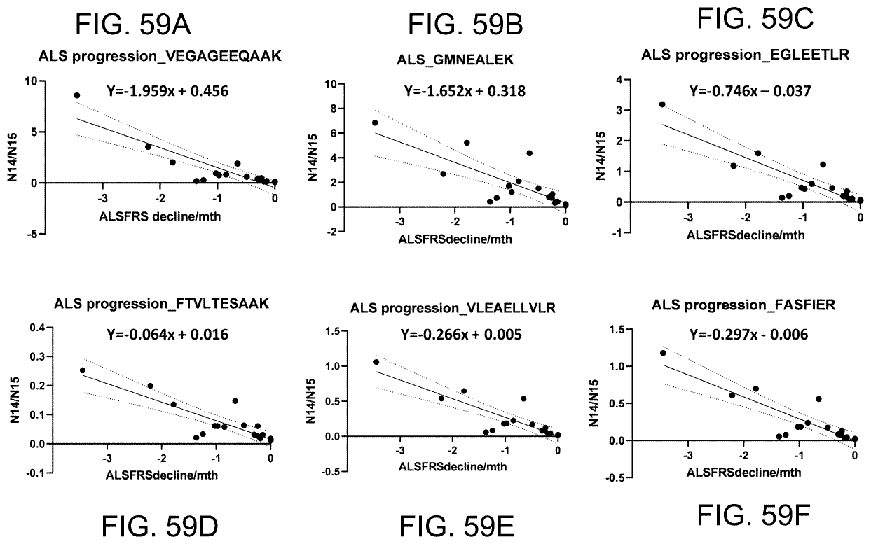

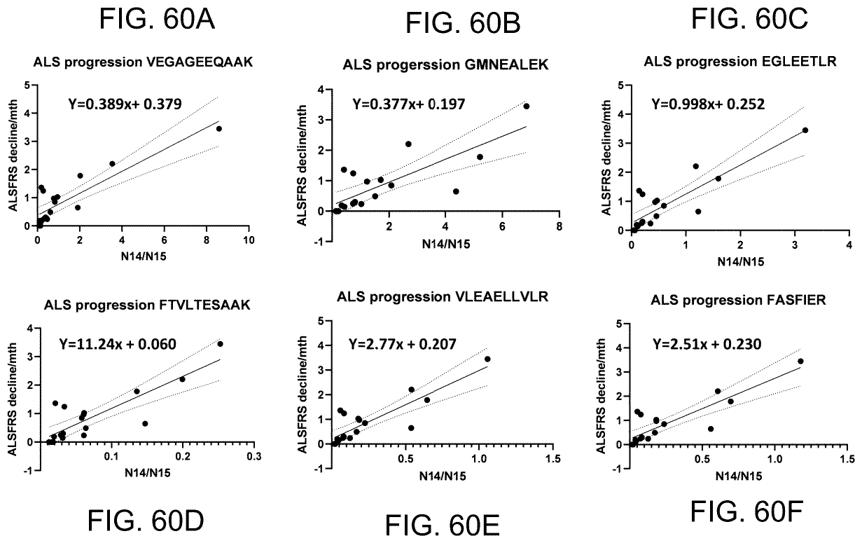

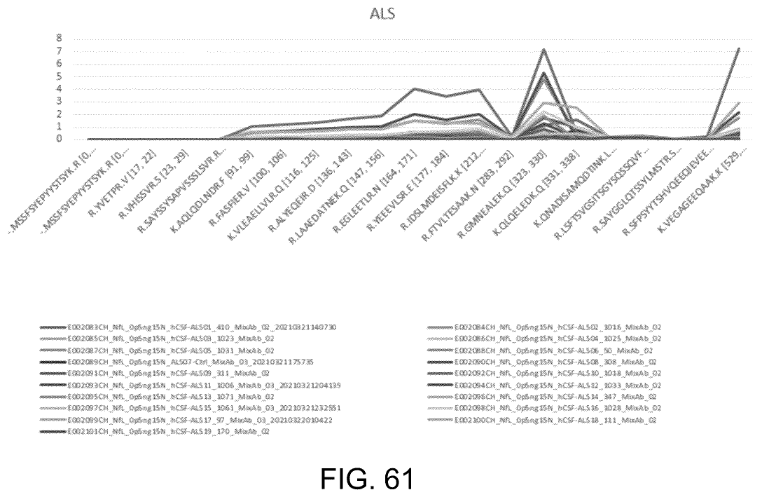

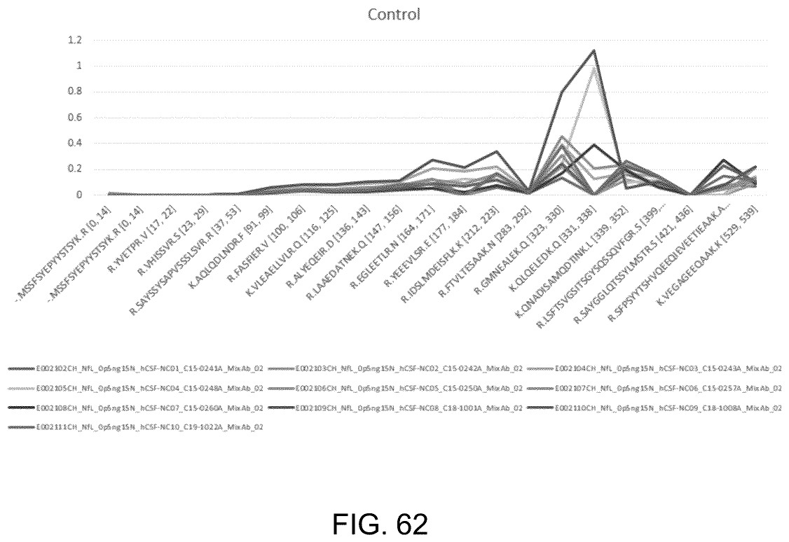

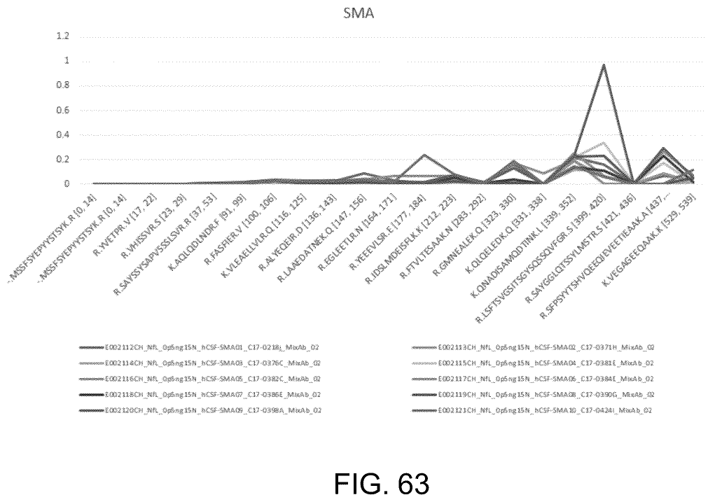

Sequential immunoprecipitation of Nfl from CSF with multiple antibodies was also performed. The CSF samples used were previously obtained from human subjects for assay development and stored at −80° C. Patient demographics for the subjects is not known (e.g., age, sex, clinical signs or symptoms of neuronal disease). Immunoprecipitation and LC-MS analysis was generally as described in Example 2, except the supernatant recovered after the first immunoprecipitation was subjected to a second round of immunoprecipitation with a second antibody, and then supernatant recovered after the second immunoprecipitation was subjected to a third round of immunoprecipitation with a third antibody. In one experiment, HJ30.2, HJ30.7, and then HJ30.11 were sequentially used for immunoprecipitation. In a second experiment, HJ30.11, HJ30.7, and HJ30.2 were sequentially used for immunoprecipitation. In these experiments, internal standard was only added to the initial CSF sample. Each experiment was performed in duplicate. A schematic of the experimental design is shown in FIG. 26. An alternative approach with internal standard also added to each post-IP supernatant prior to a subsequent round of enrichment is shown in FIG. 29.

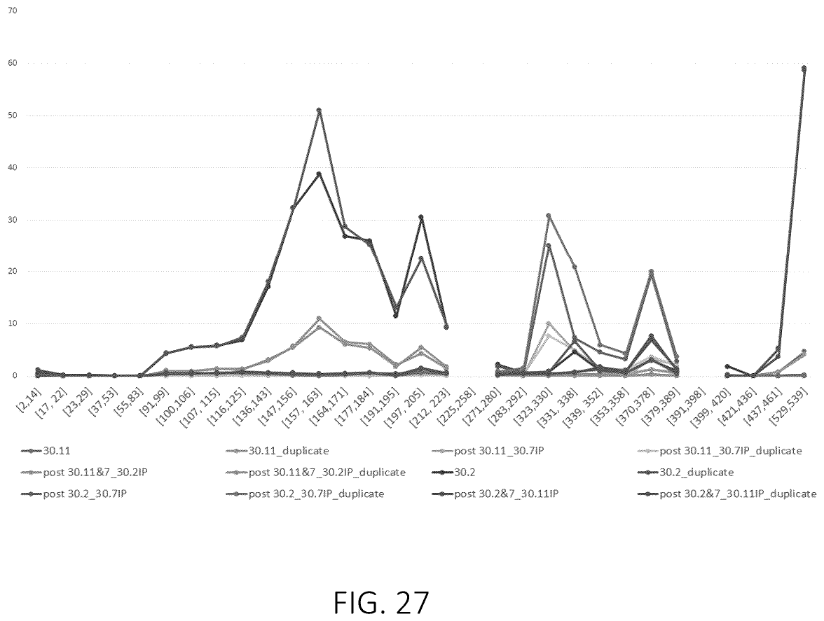

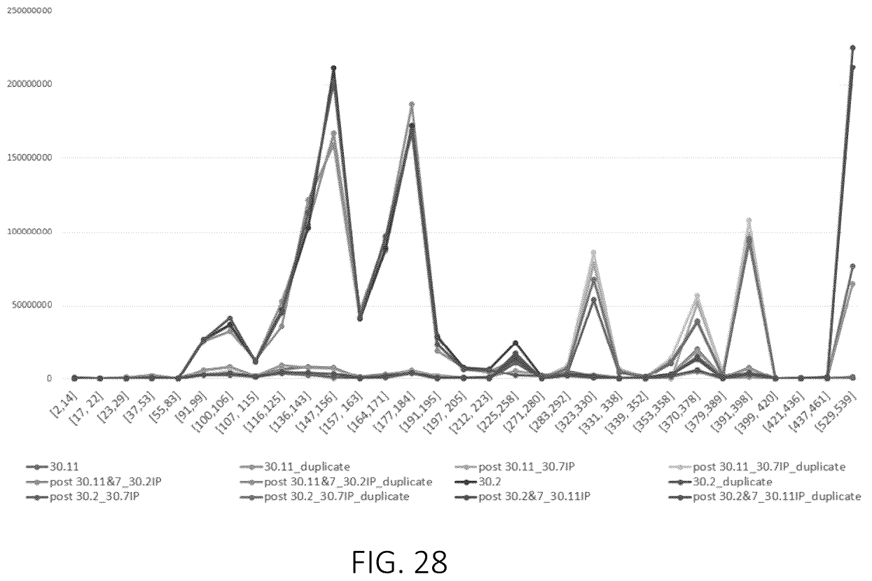

The data from both experiments are shown together in FIGS. 27-28. In FIG. 27, the y-axis is 14N/15N ratio and each point on the x-axis is a tryptic peptide of Nfl. In FIG. 28, the y-axis is 14N signal and each point on the x-axis is a tryptic peptide of Nfl. Each colored line is a different sample, as identified in the key and FIG. 26. When a reliable MS signal was not detected for a given tryptic peptide of the ISTD, no data was reported. Nfl isoforms comprising the C-terminus were more abundant following sequential enrichment using HJ30.11 at the end of the process as compared to the beginning of the process (compare “30.11” or “30.11_duplicate” to “post 30.2&7_30.11IP” or “post 30.2&7_30.11IP_duplicate” in FIG. 28). Detection of these C-terminal isoforms was negligible in the other samples analyzed. These data show that the sequential use of different epitope binding-agents enriches for different populations of Nfl isoforms.

### Example 5

In this example, Nfl isoforms from CSF samples obtained from human subjects with neuronal damage were compared to Nfl isoforms from CSF samples obtained from control human subjects. Immunoprecipitation and LC-MS analysis was generally as described in Example 2, except the supernatant recovered after a first immunoprecipitation with HJ30.13 was subjected to a second round of immunoprecipitation with HJ30.4, and then supernatant recovered after the second immunoprecipitation was subjected to a third round of immunoprecipitation with HJ30.11. Internal standard was also added to each post-IP supernatant prior to a subsequent round of enrichment. A schematic of the experimental design is shown in FIG. 29.

Data shown in FIG. 30-39 are from experiments using CSF samples obtained from human subjects with one or more biomarker for Alzheimer's disease (AD), or CSF samples obtained from control human subjects. More particularly, subjects identified as having AD were amyloid positive as evaluated by PIB-PET imaging or CSF Aβ42 levels (FIG. 30-33). All AD subjects in this cohort also had a Clinical Dementia Rating (CDR) score of ≥0.5. Control subjects had a CDR score of zero and were amyloid negative (FIG. 34-39). Each figure is an individual subject; in each figure, the blue line is Nfl isoforms immunoprecipitated by HJ30.13, the orange line is Nfl isoforms immunoprecipitated by HJ30.4, and the grey line is Nfl isoforms immunoprecipitated by HJ30.11, where the y-axis is 14N/15N ratio and each point on the x-axis is a tryptic peptide of Nfl. When a reliable MS signal was not detected for a given tryptic peptide of the ISTD, no data was reported. As summarized in FIG. 40, some parts of Nfl appear better able to differentiate AD subjects versus control subjects.

Data shown in FIG. 41 are from experiments using additional CSF samples obtained from human subjects with one or more biomarker for AD (i.e., at least amyloid positive as evaluated by PIB-PET imaging or CSF Aβ42 levels) grouped by CDR score (0, 0.5, 1, or 2), additional samples from control human subjects (amyloid negative and CDR=0), and samples obtained from human subjects who were amyloid negative but had very mild dementia (CDR 0.5) or mild dementia (CDR 1). An amyloid negative status with very mild to mild dementia may indicate other neurological disease processes (i.e., a neurological disease affecting cognition, other than AD). Due to a technical error, Nfl data from the first enrichment step were not obtainable. However, data from the second and third enrichment steps looked similar to the initial analysis of AD vs. control (amyloid negative, CDR 0). In particular, the data again show that Nfl isoforms comprising the C-terminal amino acids 530 to 540 of SEQ ID NO: 1 differentiate AD subjects from control subjects. In addition, the data from the amyloid negative subject with a mild dementia (CDR 1) suggests the Nfl isoforms detected could be useful as markers of neuronal damage due to other causes.

### Example 6

In this example, Nfl isoforms from blood samples obtained from human subjects were analyzed. Two different samples of pooled blood were used, each of which is comprised of blood previously obtained from multiple human subjects for assay development, pooled, and stored at −80° C. Immunoprecipitation and LC-MS analysis was generally as described in Example 5, except 0.5 mL of blood rather than CSF was used. As shown in FIG. 42-44, Nfl concentration in blood is lower than in CSF, but detectable. Furthermore, blood is also comprised of a plurality of Nfl isoforms, similar to CSF. Also similar to CSF, there are several truncated Nfl isoforms in blood that are comprised primarily of the C-terminus.

### Example 7

This example expands upon the previous examples by utilizing the developed antibodies which bind to various regions of NfL and characterizing NfL domains recovered by these antibodies using immunoprecipitation mass spectrometry (IP-MS) in brain tissue and CSF. The present example shows brain NfL is mainly constituted of full-length protein while CSF NfL consists of a mixture of different protein fragment species. the newly identified NfL fragment species in a discovery cohort of controls and AD were then tested, and further validated in a confirmation cohort.

Pooled CSF samples used for assay development were previously obtained from human subjects and stored at −80° C. At the time of initial collection, CSF was centrifuged at 1000×g for 10 minutes to remove cell debris and immediately frozen at −80° C. Brain samples included previously lysed samples stored at −80° C. for assay development. All AD and control CSF samples were collected during a previous study, aliquoted and stored at −80° C. The validation cohort included CSF samples from 30 symptomatic amyloid positive participants, 16 asymptomatic amyloid positive participants, 10 symptomatic amyloid negative participants, and 25 negative controls. Participant demographics are shown in Table 2.

TABLE 2
Demographics of Validation Cohort

| | Group | | | |
|---|---|---|---|---|
| | Amyloid+, | Amyloid+, | Amyloid−, | Amyloid−, |
| | CDR > 0 | CDR = 0 | CDR > 0 | CDR = 0 |
| Variable | (n = 30) | (n = 16) | (n = 10) | (n = 25) |
| Age | 74.17 ± 6.71 | 73.43 ± 6.88 | 74.30 ± 8.22 | 71.96 ± 5.51 |
| (mean ± sd) | | | | |
| Female | 9 (30.00%) | 9 (56.25%) | 2 (20.00%) | 14 (56.00%) |
| (n, %) | | | | |
| APOE4 | 19 (63.33%) | 9 (56.25%) | 2 (20.00%) | 2 (8.00%) |
| (n, %) | | | | |
| CDR Sum of | 3.42 ± 2.39 | 0.06 ± 0.17 | 2.15 ± 1.53 | 0 ± 0 |
| box | | | | |
| (mean ± sd) | | | | |
| MMSE | 25.43 ± 3.76 | 29.25 ± 0.68 | 27.80 ± 2.25 | 28.72 ± 1.54 |
| (mean ± sd) | | | | |

Both recombinant and native NfL were immunoprecipitated by adding 30 μL of a 30% (i.e., 3 mg/mL) slurry of an antibody-conjugated bead preparation and rotating the sample for 120 minutes at room temperature. The antibody-conjugated beads were magnetically separated, and the post-IP supernatant was removed. The beads were washed three times in 1 mL of 25 mM TEABC (per wash). The bound NfL was digested on-beads with 400 ng MS grade trypsin/Lys-C (Promega) for 16 hours at 37° C. Digests were loaded onto TopTip C18 (Glygen, TT2C18.96), desalted, and eluted. The eluants were dried in vacuo without heat and stored at −80° C. until analysis by liquid chromatography tandem mass spectrometry (LC-MS/MS) (see LC-MS/MS methods below.) 16 antibodies recovered full-length recombinant protein (FIG. 45). Based on peptide profiles from native NfL immunoprecipitated from pooled CSF, antibodies were determined to have epitopes against the amino-terminal portion of the rod domain, the carboxy-terminal portion of the rod domain, or the carboxy-terminus of NfL (FIG. 46). Antibodies with high recovery and NfL specificity were chosen for each of these NfL domains and used in qualitative and quantitative IP-MS assays. None of the custom antibodies recognized the N-terminus of NfL.

Three-step, sequential immunoprecipitation was used to characterize NfL in brain lysate and CSF. Antibodies targeting Coil 1A/1B of the rod domain (HJ30.13), Coil 2B of the rod domain (HJ30.4) and the tail region (HJ30.11) were used. Frozen brain lysates were thawed and a 450 μL aliquot of the thawed brain lysate was diluted 1:1000 with 1% HSA. Frozen CSF samples were thawed at room temperature, and 450 μL of the thawed CSF was transferred to a new 1.6 mL new tube for immunoprecipitation.

Both brain and CSF samples were immunoprecipitated as described above for native CSF using 30 μL of a 30% (i.e., 3 mg/mL) slurry of an antibody-conjugated bead preparation of HJ30.13 (Coil 1A/1B antibody). Washed beads were stored on ice until all samples were ready for on-bead digestion. In the second step, 20 μL of 0.5 ng/ml NFL ISTD in 50 mM TEABC was added, and NfL was immunoprecipitated a second time by adding 30 μL of a 30% (i.e., 3 mg/mL) slurry of an antibody-conjugated bead preparation of HJ30.4 (Coil 2B antibody). The remaining steps were identical to the first immunoprecipitation. 10 ng of ISTD in 50 mM TEABC was again added to the post-IP supernatant prior to the third sequential immunoprecipitation, which was performed with HJ30.11 (tail antibody). Bound NfL was digested on beads with 400 ng MS grade trypsin/Lys-C(Promega) for 16 hours at 37° C. and samples were extracted as described above.

To eliminate the need for sequential addition of ISTD, antibodies targeting Coil 1A/1B of the rod domain (HJ30.13), Coil 2B of the rod domain (HJ30.4), and the tail region (HJ30.11) were mixed 1:1:1 to generate an antibody slurry with a final concentration of 10% (i.e., 1 mg/mL) of each antibody. 25 μL of a master mix containing detergent (1% NP-40), chaotropic reagent (5 mM Guanidine), and protease inhibitors (Roche Complete Protease Inhibitor Cocktail) were added to 96 well plates. 5 μL of ISTD (0.1 ng/uL in 1% HSA; ISTD solvent and amount optimized for quantitative recovery and assay's dynamic range) were then added, followed by 450 μL of thawed CSF and 30 μL of the antibody slurry. Immunoprecipitation and on-bead digestion performed as described above.

Pooled CSF was screened to identify pools with low and high concentrations of NfL. The CSF pools with the lowest (NfL-L1) and highest (NfL-L2) NfL concentrations were selected and used to determine the assay's linear range. NfL-L2 CSF was serially diluted with NfL-L1 CSF to generate an 8 point curve with: 100%, 50%, 25%, 12.5%, 6.25%, 3.13%, and 1.56% of NfL-L2. NfL was IP′d as described above, in triplicate, for concentration. The N14/N15 ratios were determined for each of the 6 peptides in the quantitative method, and the average N14/N15 ratios of the replicates were plotted against % NfL-L2 and linear regression was performed. All 6 peptides showed good linearity across the tested NfL concentrations, with R2≥0.988 (FIG. 47). Average % CV for each peptide across the linear range was 8-12% (Table 3).

TABLE 2
Decimal % CV for IP-MS method across linear range

| % NfL-L2 | NfL101 | NfL117 | NfL165 | NfL284 | NfL324 | NfL530 |
|---|---|---|---|---|---|---|
| 0 | 0.13 | 12.69 | 18.72 | 16.00 | 8.59 | 13.37 |
| 1.56 | 0.10 | 0.10 | 0.11 | 0.05 | 0.11 | 0.08 |
| 3.125 | 0.21 | 0.15 | 0.22 | 0.15 | 0.05 | 0.07 |
| 6.25 | 0.11 | 0.10 | 0.08 | 0.02 | 0.10 | 0.18 |
| 12.5 | 0.06 | 0.08 | 0.08 | 0.06 | 0.05 | 0.06 |
| 25 | 0.04 | 0.02 | 0.06 | 0.02 | 0.03 | 0.05 |
| 50 | 0.11 | 0.05 | 0.13 | 0.07 | 0.08 | 0.01 |
| 100 | 0.16 | 0.15 | 0.06 | 0.12 | 0.16 | 0.21 |

Extracted digests were reconstituted with 25 μL of 0.1% Formic acid/0% ACN. A 4.5 uL aliquot of each digest was then injected into nano-Acquity LC for MS analysis. The nano-Acquity LC (Waters Corporation, Milford, MA, USA) was fitted with HSS T3 75 μm×100 μm, 1.8 μm column and a flow rate of 0.5 μL/min of a gradient of solution A and B was used to separate the peptides. Solution A was composed of 0.1% formic acid in MS grade water and solution B was composed of 0.1% formic acid in acetonitrile. Samples were analyzed in positive ion mode, with a spray voltage of 2,200V and ion transfer tube temperature of 275° C. Data were collected with parallel reaction monitoring (PRM) for endogenous (N14) and isotopically labeled (Lys, Arg: 13C 15N) peptides. Tryptic peptides specific to NfL were identified via Blast search, and those with good ionization were included in the qualitative PRM, designed to optimize sequence coverage. The quantitative method was optimized for assay precision, and multiplexing was reduced to analysis of 6 NfL peptides across various NfL domains and their corresponding ISTDs (Table 4).

TABLE 3
Quantitative MS Peptide List

| | | | | Fragment | |
|---|---|---|---|---|---|
| | | | | ion used | |
| | Amino | | Precursor | for | Fragment |
| Peptide sequence | acid # | Z | m/z | quantitation | m/z |
| FASFIER (light) | 100-106 | 2 | 435.2294 | y5+ | 651.3461 |
| (SEQ ID NO: 7) | | | | | |
| FASFIER (C1315N-ISTD) | 100-106 | 2 | 440.2336 | y5+ | 661.3543 |
| (SEQ ID NO: 7) | | | | | |
| VLEAELLVLR (light) | 116-125 | 2 | 577.8608 | y8+ | 942.5619 |
| (SEQ ID NO: 9) | | | | | |
| VLEAELLVLR (C1315N-ISTD) | 116-125 | 2 | 582.8649 | y8+ | 952.5701 |
| (SEQ ID NO: 9) | | | | | |
| EGLEETLR (light) | 164-171 | 2 | 473.7456 | y4+ | 518.2933 |
| (SEQ ID NO: 13) | | | | | |
| EGLEETLR (C1315N-ISTD) | 164-171 | 2 | 478.7498 | y4+ | 528.3016 |
| (SEQ ID NO: 13) | | | | | |
| FTVLTESAAK (light) | 283-292 | 2 | 533.7926 | y8+ | 818.4618 |
| (SEQ ID NO: 19) | | | | | |
| FTVLTESAAK (C1315N-ISTD) | 283-292 | 2 | 537.7997 | y8+ | 826.476 |
| (SEQ ID NO: 19) | | | | | |
| GMNEALEK (light) | 323-330 | 2 | 446.2157 | y6+ | 703.3621 |
| (SEQ ID NO: 20) | | | | | |
| GMNEALEK (C1315N-ISTD) | 323-330 | 2 | 450.2228 | y6+ | 711.3763 |
| (SEQ ID NO: 20) | | | | | |
| VEGAGEEQAAK (light) | 529-539 | 2 | 544.7646 | y9+ | 860.4108 |
| (SEQ ID NO: 29) | | | | | |
| VEGAGEEQAAK (C1315N-ISTD) | 529-539 | 2 | 548.7717 | y9+ | 868.425 |
| (SEQ ID NO: 29) | | | | | |

The validation cohort consisted of 81 CSF samples previously collected from individuals with AD dementia (amyloid positive, CDR 0-2), non-AD dementia (amyloid negative, CDR 0.5-1), and healthy controls (amyloid negative, CDR 0). Amyloid positivity was previously determined by Aβ42/Aβ 40 ratio (Patterson et al., 2015). For each CSF sample, six NfL peptides, corresponding to four different domains of NfL (Coil 1A, Coil 1B, Coil 2B, and Tail), were measured using the quantitative IP-MS method described above. NfL was also measured via commercial ELISA kit (UMAN Diagnostics) according to manufacturer's specifications. Briefly, for ELISA measurement, CSF samples were thawed on wet ice and vortexed. Samples were then diluted 2× with the provided sample diluent in a96 well pre-plate and mixed prior to transferring to the assay plate.

To determine the relationship of soluble NfL species to AD clinical, cognitive, imaging and biomarker measures, correlation analysis was performed between each NfL region (IP-MS) and previously obtained biomarker data. The following measures were evaluated: age, CDR-global and CDR Sum of Boxes (CDR-SB), Mini-Mental State Exam (MMSE), amyloid plaque imaging (PET PiB), CSF Aβ42/Aβ40, CSF total tau (t-tau), CSF ptau 181 and ptau181/tau181, CSF ptau205 and ptau205/tau205, and CSF ptau217 and ptau217/tau217.

Twenty-three monoclonal antibodies were generated against NfL and evaluated for their ability to immunoprecipitate full-length rec-NfL, NfL from brain lysate, and NfL from pooled CSF. Antibodies were characterized by the Nfl domain they targeted, their IP-efficiency, and their specificity. Representative antibodies for each NfL domain were selected and used for further assay development. Using antibodies targeting various NfL domains, we determined that multiple NfL species exist in CSF (FIG. 48). To better elucidate the Nfl species in CSF, pooled CSF samples were sequentially immunoprecipitated starting with an antibody targeting the Coil 1A/1B (approximately aa93-252, HJ30.13), followed by an antibody targeting the Coil 2B (approximately aa272-396, HJ30.4), and finally with an antibody targeting the C-terminus of the tail region (aa 520-550, HJ30.11). Based on these protein profiles identified a minimum of three major NfL fragment species in CSF, though it is likely that multiple variations of these species exist. These include two different N-terminal and C-terminal truncations containing rod domains enriched by HJ30.13 (aa92 through at least aa224, with possible variations extending through aa360) and HJ30.4 (aa324 through aa360), as well as a C-terminal fragment containing the tail of NfL (enriched by HJ30.11, containing aa530 through at least aa540). No N-terminal fragments were recovered and full length NfL was not present in quantifiable concentrations in CSF (FIG. 49A-49B).

In contrast to the highly fragmented protein in CSF, brain tissue homogenate contained mostly full-length NfL. To determine if any truncated isoforms were also present in brain, the same sequential immunoprecipitation on human brain tissue was performed. While most brain NfL appeared to be full-length, a C-terminal fragment of tail subdomain B containing at least amino acids 530-540 was also observed (FIG. 49A, 49C), similar to the fragment identified in CSF. A fragment containing aa165-224 appears to be enriched by HJ30.13. No additional NfL fragments were enriched in brain during the second IP (HJ30.4, Coil 2B of rod domain.)

The sequential IP-MS method initially tested on experimental, pooled CSF was repeated on CSF samples from a discovery cohort of Alzheimer's disease dementia (AD, N=4) and healthy controls (N=6). The peptides observed in both clinical groups were similar to those observed in the pooled CSF, but there were increased amounts of the three major NfL species in AD compared to controls (FIG. 50). Additionally, some peptides appeared to better than others at differentiating AD and control samples. The most prominent difference was observed for the NfL530 (tryptic peptide VEGAGEEQAAK (SEQ ID NO: 29), aa530-540) in the C-terminal tail and tryptic peptide GADEAALAR (SEQ ID NO: 16), within coil 1B of the rod domain.

In order to better compare CSF NfL species in AD, non-AD dementia, and healthy controls, we developed a quantitative NfL assay to reliably measure select regions across multiple NfL species. In order to improve precision, multiplexing in our quantitative method was reduced to measure 6 peptides across the various NfL domains and their corresponding internal standards. IP-MS quantitative method was then applied to measure specific CSF NfL species in a validation cohort of 81 AD and control samples (30 amyloid positive, CDR>0; 16 amyloid positive, CDR=0; 10 amyloid negative, CDR=0; 25 amyloid negative, CDR=0. Table 2). CSF NfL concentrations were also quantified using the gold standard Uman NfL immunoassay for comparison.

Consistent with sequential IP results of the discovery cohort (FIG. 51), increases in Nfl concentrations were confirmed in a confirmation cohort of symptomatic, amyloid positive individuals (N=30) compared to amyloid negative healthy controls (N=25) (FIG. 52). Interestingly, the difference between groups was larger for some regions than for others, with the biggest differences observed in Coil 2B of the rod domain (NfL324; GMNEALEK (SEQ ID NO: 20), aa324-331) and in the C-terminus of the tail (NfL530; VEGAGEEQAAK (SEQ ID NO: 29), aa530-540.) NfL324 was 1.5 fold increased in AD compared to control (P=0.008, FIG. 52F) and NfL530 was 1.7 fold increased (P=0.002, FIG. 52G).

NfL immunoassay was 1.4 fold increased in AD compared to controls). It had the strongest correlation with NfL324 peptide concentrations (R2=0.84) suggesting Uman immunoassay targets CSF NfL fragments containing the Coil 2B region. Importantly, correlations between the immunoassay and other investigated peptides were lower for NfL101, 117, 165 and 530 (R2 ranging from 0.34 to 0.49) and no correlation was found with NfL284 (FIG. 52).

As NfL is a marker of general neurodegeneration and not specific to AD, it was hypothesized that NfL would be increased regardless of the presence of amyloid plaques in those with clinical dementia and neurodegeneration. The correlation between CDR-SB (a clinical measure of dementia severity) and NfL species were evaluated for amyloid positive and amyloid negative samples (FIG. 53). While correlation was slightly higher for some NfL species in the amyloid positive group than the amyloid negative group (NfL101, NfL117, NfL 165 and NfL324) correlation was minimal or low for all NfL species in both groups. Correlation between NfL530 and CDR-SB was not significantly non-zero for either group.

Spearman correlation analysis was also performed between each of the 6 quantified NfL peptides and additional previously measured biomarkers and clinical measures including: general markers of clinical dementia (CDR-SB, MMSE), biomarkers of amyloid plaques (PET PIB, CSF Aβ42/Aβ40) and tau biomarkers (CSF total-tau, CSF phospho-tau immunoassay, and mass spectrometry measures of CSF ptau 181, 205 and 217 occupancy); FIG. 54, Table 5. The goal of this analysis was to form hypotheses about the biology of the different NfL species in general neurodegeneration compared to disease specific neurodegeneration. The strongest correlations were observed between peptides within Coil1A (NfL101 and NfL117, r=0.99) and Coil 1B of the rod domain (NfL 101 and NfL 165, r=0.98; NfL 117 and NfL 165 r=0.98). Peptides in coil 2B of the rod domain have similar, but slightly lower correlation with the coil 1A peptides (NfL 101 and NfL284, r=0.89; NfL 101 and NfL324, r=0.87; NfL 117 and NfL284, r=0.90; NfL117 and NfL324, r=0.88). The correlation between the C-terminal tail peptide and coil 1A is the lowest among the NfL peptides investigated (NfL 101 and NfL530, r=0.75; NfL 117 and NfL530, r=0.76). Interestingly, the most c-terminal peptides measured (NfL324 and NfL530) have the highest correlation between disease biomarkers and NfL. The moderate correlation between NfL324 or NfL530 and ptau 181, 205, or 217 ranges from r=0.45 to 0.49. The correlation between the same NfL peptides and tTau is r=0.42 to 0.43. Correlation with CSF Aβ42/Aβ40 and CSF NfL530 was lower at −0.37. (FIG. 53, Table 4).

TABLE 5
Spearman Correlation table

| var1 | var2 | Correlation | p. value | 95% CI |
|---|---|---|---|---|
| Age | CDR Global | 0.07 | 0.5066 | (−0.15, 0.29) |
| Age | CDR Sum of box | 0.09 | 0.4324 | (−0.13, 0.3) |
| Age | MMSE | −0.19 | 0.0976 | (−0.39, 0.03) |
| Age | Amyloid PET (PiB) | 0.47 | 5.00E−04 | (0.22, 0.66) |
| Age | CSF Aß42/Aß40 (MS) | −0.17 | 0.1386 | (−0.37, 0.05) |
| Age | CSF tTau (ELISA) | 0.19 | 0.1007 | (−0.04, 0.4) |
| Age | CSF pTau181 (ELISA) | 0.18 | 0.1128 | (−0.04, 0.4) |
| Age | CSF pTau181% (IPMS) | 0.07 | 0.5519 | (−0.16, 0.28) |
| Age | CSF pTau205% (IPMS) | 0.12 | 0.2829 | (−0.1, 0.33) |
| Age | CSF pTau217% (IPMS) | 0.22 | 0.0558 | (−0.01, 0.42) |
| Age | CSF pTau181, ng/mL (IPMS) | 0.09 | 0.4359 | (−0.13, 0.3) |
| Age | CSF pTau205, ng/mL (IPMS) | 0.13 | 0.2544 | (−0.09, 0.34) |
| Age | CSF pTau217, ng/mL (IPMS) | 0.2 | 0.0754 | (−0.02, 0.4) |
| Age | NfL ELISA | 0.36 | 0.0011 | (0.15, 0.54) |
| Age | NfL101 | 0.24 | 0.0293 | (0.03, 0.44) |
| Age | NfL117 | 0.23 | 0.0411 | (0.01, 0.42) |
| Age | NfL165 | 0.25 | 0.0224 | (0.04, 0.45) |
| Age | NfL284 | 0.19 | 0.0834 | (−0.03, 0.4) |
| Age | NfL324 | 0.29 | 0.0091 | (0.07, 0.48) |
| Age | NfL530 | 0.38 | 4.00E−04 | (0.18, 0.56) |
| CDR Global | CDR Sum of box | 0.96 | <0.0001 | (0.94, 0.97) |
| CDR Global | MMSE | −0.56 | <0.0001 | (−0.69, −0.39) |
| CDR Global | Amyloid PET (PiB) | 0.38 | 0.0064 | (0.11, 0.6) |
| CDR Global | CSF Aß42/Aß40 (MS) | −0.39 | 3.00E−04 | (−0.56, −0.19) |
| CDR Global | CSF tTau (ELISA) | 0.44 | 1.00E−04 | (0.23, 0.6) |
| CDR Global | CSF pTau181 (ELISA) | 0.39 | 5.00E−04 | (0.18, 0.57) |
| CDR Global | CSF pTau181% (IPMS) | 0.43 | 1.00E−04 | (0.23, 0.6) |
| CDR Global | CSF pTau205% (IPMS) | 0.55 | <0.0001 | (0.38, 0.69) |
| CDR Global | CSF pTau217% (IPMS) | 0.51 | <0.0001 | (0.33, 0.66) |
| CDR Global | CSF pTau181, ng/mL (IPMS) | 0.4 | 2.00E−04 | (0.2, 0.57) |
| CDR Global | CSF pTau205, ng/mL (IPMS) | 0.5 | <0.0001 | (0.32, 0.65) |
| CDR Global | CSF pTau217, ng/mL (IPMS) | 0.47 | <0.0001 | (0.28, 0.63) |
| CDR Global | NfL ELISA | 0.35 | 0.0018 | (0.14, 0.53) |
| CDR Global | NfL101 | 0.3 | 0.0057 | (0.09, 0.49) |
| CDR Global | NfL117 | 0.31 | 0.0049 | (0.1, 0.49) |
| CDR Global | NfL165 | 0.33 | 0.0026 | (0.12, 0.51) |
| CDR Global | NfL284 | 0.27 | 0.0142 | (0.06, 0.46) |
| CDR Global | NfL324 | 0.36 | 0.0012 | (0.15, 0.53) |
| CDR Global | NfL530 | 0.31 | 0.005 | (0.1, 0.49) |
| CDR Sum of box | MMSE | −0.55 | <0.0001 | (−0.68, −0.37) |
| CDR Sum of box | Amyloid PET (PiB) | 0.34 | 0.0147 | (0.07, 0.57) |
| CDR Sum of box | CSF Aß42/Aß40 (MS) | −0.46 | <0.0001 | (−0.61, −0.27) |
| CDR Sum of box | CSF tTau (ELISA) | 0.48 | <0.0001 | (0.29, 0.64) |
| CDR Sum of box | CSF pTau181 (ELISA) | 0.46 | <0.0001 | (0.26, 0.62) |
| CDR Sum of box | CSF pTau181% (IPMS) | 0.48 | <0.0001 | (0.29, 0.63) |
| CDR Sum of box | CSF pTau205% (IPMS) | 0.57 | <0.0001 | (0.4, 0.71) |
| CDR Sum of box | CSF pTau217% (IPMS) | 0.55 | <0.0001 | (0.38, 0.69) |
| CDR Sum of box | CSF pTau181, ng/mL (IPMS) | 0.43 | 1.00E−04 | (0.23, 0.59) |
| CDR Sum of box | CSF pTau205, ng/mL (IPMS) | 0.52 | <0.0001 | (0.35, 0.67) |
| CDR Sum of box | CSF pTau217, ng/mL (IPMS) | 0.5 | <0.0001 | (0.32, 0.65) |
| CDR Sum of box | NfL ELISA | 0.37 | 9.00E−04 | (0.16, 0.54) |
| CDR Sum of box | NfL101 | 0.34 | 0.002 | (0.13, 0.52) |
| CDR Sum of box | NfL117 | 0.34 | 0.0016 | (0.14, 0.52) |
| CDR Sum of box | NfL165 | 0.37 | 8.00E−04 | (0.16, 0.54) |
| CDR Sum of box | NfL284 | 0.3 | 0.0059 | (0.09, 0.49) |
| CDR Sum of box | NfL324 | 0.38 | 4.00E−04 | (0.18, 0.56) |
| CDR Sum of box | NfL530 | 0.33 | 0.0025 | (0.12, 0.51) |
| MMSE | Amyloid PET (PiB) | −0.43 | 0.0017 | (−0.64, −0.18) |
| MMSE | CSF Aß42/Aß40 (MS) | 0.22 | 0.0459 | (0, 0.42) |
| MMSE | CSF tTau (ELISA) | −0.31 | 0.0071 | (−0.5, −0.09) |
| MMSE | CSF pTau181 (ELISA) | −0.25 | 0.0282 | (−0.45, −0.03) |
| MMSE | CSF pTau181% (IPMS) | −0.38 | 5.00E−04 | (−0.56, −0.18) |
| MMSE | CSF pTau205% (IPMS) | −0.46 | <0.0001 | (−0.62, −0.27) |
| MMSE | CSF pTau217% (IPMS) | −0.46 | <0.0001 | (−0.62, −0.26) |
| MMSE | CSF pTau181, ng/mL (IPMS) | −0.28 | 0.0102 | (−0.47, −0.07) |
| MMSE | CSF pTau205, ng/mL (IPMS) | −0.38 | 5.00E−04 | (−0.55, −0.18) |
| MMSE | CSF pTau217, ng/mL (IPMS) | −0.37 | 6.00E−04 | (−0.55, −0.17) |
| MMSE | NfL ELISA | −0.24 | 0.0332 | (−0.44, −0.02) |
| MMSE | NfL101 | −0.1 | 0.3894 | (−0.31, 0.12) |
| MMSE | NfL117 | −0.1 | 0.3956 | (−0.31, 0.13) |
| MMSE | NfL165 | −0.14 | 0.2266 | (−0.34, 0.09) |
| MMSE | NfL284 | −0.03 | 0.8157 | (−0.24, 0.19) |
| MMSE | NfL324 | −0.14 | 0.231 | (−0.34, 0.09) |
| MMSE | NfL530 | −0.06 | 0.5854 | (−0.28, 0.16) |
| Amyloid PET (PiB) | CSF Aß42/Aß40 (MS) | −0.63 | <0.0001 | (−0.77, −0.42) |
| Amyloid PET (PiB) | CSF tTau (ELISA) | 0.41 | 0.0035 | (0.14, 0.62) |
| Amyloid PET (PiB) | CSF pTau181 (ELISA) | 0.38 | 0.0067 | (0.11, 0.6) |
| Amyloid PET (PiB) | CSF pTau181% (IPMS) | 0.61 | <0.0001 | (0.39, 0.76) |
| Amyloid PET (PiB) | CSF pTau205% (IPMS) | 0.61 | <0.0001 | (0.4, 0.76) |
| Amyloid PET (PiB) | CSF pTau217% (IPMS) | 0.77 | <0.0001 | (0.63, 0.87) |
| Amyloid PET (PiB) | CSF pTau181, ng/mL (IPMS) | 0.41 | 0.0028 | (0.15, 0.62) |
| Amyloid PET (PiB) | CSF pTau205, ng/mL (IPMS) | 0.54 | <0.0001 | (0.31, 0.71) |
| Amyloid PET (PiB) | CSF pTau217, ng/mL (IPMS) | 0.72 | <0.0001 | (0.56, 0.83) |
| Amyloid PET (PiB) | NfL ELISA | 0.31 | 0.0298 | (0.03, 0.54) |
| Amyloid PET (PiB) | NfL101 | 0.05 | 0.7195 | (−0.23, 0.33) |
| Amyloid PET (PiB) | NfL117 | 0.05 | 0.7115 | (−0.23, 0.33) |
| Amyloid PET (PiB) | NfL165 | 0.08 | 0.5918 | (−0.21, 0.35) |
| Amyloid PET (PiB) | NfL284 | 0.02 | 0.9001 | (−0.26, 0.3) |
| Amyloid PET (PiB) | NfL324 | 0.22 | 0.1266 | (−0.06, 0.47) |
| Amyloid PET (PiB) | NfL530 | 0.23 | 0.1017 | (−0.05, 0.48) |
| CSF Aß42/Aß40 (MS) | CSF tTau (ELISA) | −0.67 | <0.0001 | (−0.78, −0.53) |
| CSF Aß42/Aß40 (MS) | CSF pTau181 (ELISA) | −0.6 | <0.0001 | (−0.73, −0.43) |
| CSF Aß42/Aß40 (MS) | CSF pTau181% (IPMS) | −0.77 | <0.0001 | (−0.84, −0.66) |
| CSF Aß42/Aß40 (MS) | CSF pTau205% (IPMS) | −0.56 | <0.0001 | (−0.69, −0.38) |
| CSF Aß42/Aß40 (MS) | CSF pTau217% (IPMS) | −0.81 | <0.0001 | (−0.88, −0.72) |
| CSF Aß42/Aß40 (MS) | CSF pTau181, ng/mL (IPMS) | −0.59 | <0.0001 | (−0.71, −0.42) |
| CSF Aß42/Aß40 (MS) | CSF pTau205, ng/mL (IPMS) | −0.6 | <0.0001 | (−0.72, −0.44) |
| CSF Aß42/Aß40 (MS) | CSF pTau217, ng/mL (IPMS) | −0.76 | <0.0001 | (−0.84, −0.65) |
| CSF Aß42/Aß40 (MS) | NfL ELISA | −0.31 | 0.0054 | (−0.5, −0.1) |
| CSF Aß42/Aß40 (MS) | NfL101 | −0.22 | 0.0466 | (−0.42, 0) |
| CSF Aß42/Aß40 (MS) | NfL117 | −0.22 | 0.0435 | (−0.42, −0.01) |
| CSF Aß42/Aß40 (MS) | NfL165 | −0.24 | 0.0345 | (−0.43, −0.02) |
| CSF Aß42/Aß40 (MS) | NfL284 | −0.16 | 0.1418 | (−0.37, 0.06) |
| CSF Aß42/Aß40 (MS) | NfL324 | −0.3 | 0.0065 | (−0.49, −0.09) |
| CSF Aß42/Aß40 (MS) | NfL530 | −0.37 | 6.00E−04 | (−0.55, −0.17) |
| CSF tTau (ELISA) | CSF pTau181 (ELISA) | 0.82 | <0.0001 | (0.72, 0.88) |
| CSF tTau (ELISA) | CSF pTau181% (IPMS) | 0.68 | <0.0001 | (0.53, 0.79) |
| CSF tTau (ELISA) | CSF pTau205% (IPMS) | 0.57 | <0.0001 | (0.39, 0.71) |
| CSF tTau (ELISA) | CSF pTau217% (IPMS) | 0.7 | <0.0001 | (0.56, 0.8) |
| CSF tTau (ELISA) | CSF pTau181, ng/mL (IPMS) | 0.77 | <0.0001 | (0.66, 0.85) |
| CSF tTau (ELISA) | CSF pTau205, ng/mL (IPMS) | 0.75 | <0.0001 | (0.63, 0.84) |
| CSF tTau (ELISA) | CSF pTau217, ng/mL (IPMS) | 0.75 | <0.0001 | (0.64, 0.84) |
| CSF tTau (ELISA) | NfL ELISA | 0.48 | <0.0001 | (0.28, 0.64) |
| CSF tTau (ELISA) | NfL101 | 0.3 | 0.008 | (0.08, 0.5) |
| CSF tTau (ELISA) | NfL117 | 0.31 | 0.0072 | (0.09, 0.5) |
| CSF tTau (ELISA) | NfL165 | 0.32 | 0.0045 | (0.11, 0.51) |
| CSF tTau (ELISA) | NfL284 | 0.19 | 0.0995 | (−0.04, 0.4) |
| CSF tTau (ELISA) | NfL324 | 0.43 | 1.00E−04 | (0.22, 0.6) |
| CSF tTau (ELISA) | NfL530 | 0.42 | 2.00E−04 | (0.21, 0.59) |
| CSF pTau181 (ELISA) | CSF pTau181% (IPMS) | 0.58 | <0.0001 | (0.4, 0.72) |
| CSF pTau181 (ELISA) | CSF pTau205% (IPMS) | 0.42 | 2.00E−04 | (0.21, 0.59) |
| CSF pTau181 (ELISA) | CSF pTau217% (IPMS) | 0.56 | <0.0001 | (0.37, 0.7) |
| CSF pTau181 (ELISA) | CSF pTau181, ng/mL (IPMS) | 0.68 | <0.0001 | (0.54, 0.79) |
| CSF pTau181 (ELISA) | CSF pTau205, ng/mL (IPMS) | 0.62 | <0.0001 | (0.46, 0.74) |
| CSF pTau181 (ELISA) | CSF pTau217, ng/mL (IPMS) | 0.63 | <0.0001 | (0.47, 0.75) |
| CSF pTau181 (ELISA) | NfL ELISA | 0.45 | 1.00E−04 | (0.25, 0.62) |
| CSF pTau181 (ELISA) | NfL101 | 0.34 | 0.0025 | (0.13, 0.53) |
| CSF pTau181 (ELISA) | NfL117 | 0.34 | 0.0026 | (0.13, 0.53) |
| CSF pTau181 (ELISA) | NfL165 | 0.34 | 0.0025 | (0.13, 0.53) |
| CSF pTau181 (ELISA) | NfL284 | 0.25 | 0.0339 | (0.02, 0.45) |
| CSF pTau181 (ELISA) | NfL324 | 0.4 | 4.00E−04 | (0.19, 0.58) |
| CSF pTau181 (ELISA) | NfL530 | 0.4 | 4.00E−04 | (0.19, 0.57) |
| CSF pTau181% (IPMS) | CSF pTau205% (IPMS) | 0.71 | <0.0001 | (0.57, 0.8) |
| CSF pTau181% (IPMS) | CSF pTau217% (IPMS) | 0.91 | <0.0001 | (0.87, 0.94) |
| CSF pTau181% (IPMS) | CSF pTau181, ng/mL (IPMS) | 0.8 | <0.0001 | (0.7, 0.87) |
| CSF pTau181% (IPMS) | CSF pTau205, ng/mL (IPMS) | 0.79 | <0.0001 | (0.69, 0.86) |
| CSF pTau181% (IPMS) | CSF pTau217, ng/mL (IPMS) | 0.88 | <0.0001 | (0.82, 0.92) |
| CSF pTau181% (IPMS) | NfL ELISA | 0.29 | 0.0106 | (0.07, 0.48) |
| CSF pTau181% (IPMS) | NfL101 | 0.16 | 0.1579 | (−0.06, 0.37) |
| CSF pTau181% (IPMS) | NfL117 | 0.17 | 0.1345 | (−0.05, 0.38) |
| CSF pTau181% (IPMS) | NfL165 | 0.17 | 0.1274 | (−0.05, 0.38) |
| CSF pTau181% (IPMS) | NfL284 | 0.09 | 0.4451 | (−0.14, 0.3) |
| CSF pTau181% (IPMS) | NfL324 | 0.25 | 0.0276 | (0.03, 0.45) |
| CSF pTau181% (IPMS) | NfL530 | 0.33 | 0.0029 | (0.12, 0.51) |
| CSF pTau205% (IPMS) | CSF pTau217% (IPMS) | 0.78 | <0.0001 | (0.68, 0.86) |
| CSF pTau205% (IPMS) | CSF pTau181, ng/mL (IPMS) | 0.55 | <0.0001 | (0.38, 0.69) |
| CSF pTau205% (IPMS) | CSF pTau205, ng/mL (IPMS) | 0.85 | <0.0001 | (0.78, 0.9) |
| CSF pTau205% (IPMS) | CSF pTau217, ng/mL (IPMS) | 0.7 | <0.0001 | (0.57, 0.8) |
| CSF pTau205% (IPMS) | NfL ELISA | 0.37 | 9.00E−04 | (0.16, 0.55) |
| CSF pTau205% (IPMS) | NfL101 | 0.22 | 0.0498 | (0, 0.42) |
| CSF pTau205% (IPMS) | NfL117 | 0.23 | 0.0402 | (0.01, 0.43) |
| CSF pTau205% (IPMS) | NfL165 | 0.26 | 0.0223 | (0.04, 0.45) |
| CSF pTau205% (IPMS) | NfL284 | 0.15 | 0.1792 | (−0.07, 0.36) |
| CSF pTau205% (IPMS) | NfL324 | 0.33 | 0.0033 | (0.11, 0.51) |
| CSF pTau205% (IPMS) | NfL530 | 0.34 | 0.0024 | (0.12, 0.52) |
| CSF pTau217% (IPMS) | CSF pTau181, ng/mL (IPMS) | 0.75 | <0.0001 | (0.63, 0.83) |
| CSF pTau217% (IPMS) | CSF pTau205, ng/mL (IPMS) | 0.82 | <0.0001 | (0.73, 0.88) |
| CSF pTau217% (IPMS) | CSF pTau217, ng/mL (IPMS) | 0.94 | <0.0001 | (0.91, 0.96) |
| CSF pTau217% (IPMS) | NfL ELISA | 0.41 | 2.00E−04 | (0.2, 0.58) |
| CSF pTau217% (IPMS) | NfL101 | 0.26 | 0.0197 | (0.04, 0.46) |
| CSF pTau217% (IPMS) | NfL117 | 0.27 | 0.0144 | (0.06, 0.47) |
| CSF pTau217% (IPMS) | NfL165 | 0.29 | 0.0104 | (0.07, 0.48) |
| CSF pTau217% (IPMS) | NfL284 | 0.18 | 0.1131 | (−0.04, 0.39) |
| CSF pTau217% (IPMS) | NfL324 | 0.38 | 6.00E−04 | (0.17, 0.55) |
| CSF pTau217% (IPMS) | NfL530 | 0.44 | <0.0001 | (0.25, 0.61) |
| CSF pTau181, ng/mL (IPMS) | CSF pTau205, ng/mL (IPMS) | 0.89 | <0.0001 | (0.83, 0.92) |
| CSF pTau181, ng/mL (IPMS) | CSF pTau217, ng/mL (IPMS) | 0.91 | <0.0001 | (0.86, 0.94) |
| CSF pTau181, ng/mL (IPMS) | NfL ELISA | 0.53 | <0.0001 | (0.35, 0.67) |
| CSF pTau181, ng/mL (IPMS) | NfL101 | 0.29 | 0.0076 | (0.08, 0.48) |
| CSF pTau181, ng/mL (IPMS) | NfL117 | 0.3 | 0.0063 | (0.09, 0.49) |
| CSF pTau181, ng/mL (IPMS) | NfL165 | 0.32 | 0.0036 | (0.11, 0.5) |
| CSF pTau181, ng/mL (IPMS) | NfL284 | 0.16 | 0.1446 | (−0.06, 0.37) |
| CSF pTau181, ng/mL (IPMS) | NfL324 | 0.45 | <0.0001 | (0.26, 0.61) |
| CSF pTau181, ng/mL (IPMS) | NfL530 | 0.48 | <0.0001 | (0.3, 0.63) |
| CSF pTau205, ng/mL (IPMS) | CSF pTau217, ng/mL (IPMS) | 0.9 | <0.0001 | (0.85, 0.93) |
| CSF pTau205, ng/mL (IPMS) | NfL ELISA | 0.53 | <0.0001 | (0.36, 0.68) |
| CSF pTau205, ng/mL (IPMS) | NfL101 | 0.32 | 0.004 | (0.1, 0.5) |
| CSF pTau205, ng/mL (IPMS) | NfL117 | 0.32 | 0.0033 | (0.11, 0.51) |
| CSF pTau205, ng/mL (IPMS) | NfL165 | 0.35 | 0.0016 | (0.14, 0.52) |
| CSF pTau205, ng/mL (IPMS) | NfL284 | 0.19 | 0.0907 | (−0.03, 0.39) |
| CSF pTau205, ng/mL (IPMS) | NfL324 | 0.47 | <0.0001 | (0.28, 0.63) |
| CSF pTau205, ng/mL (IPMS) | NfL530 | 0.47 | <0.0001 | (0.28, 0.62) |
| CSF pTau217, ng/mL (IPMS) | NfL ELISA | 0.51 | <0.0001 | (0.33, 0.66) |
| CSF pTau217, ng/mL (IPMS) | NfL101 | 0.3 | 0.0061 | (0.09, 0.49) |
| CSF pTau217, ng/mL (IPMS) | NfL117 | 0.31 | 0.0046 | (0.1, 0.5) |
| CSF pTau217, ng/mL (IPMS) | NfL165 | 0.32 | 0.0031 | (0.11, 0.51) |
| CSF pTau217, ng/mL (IPMS) | NfL284 | 0.18 | 0.1179 | (−0.04, 0.38) |
| CSF pTau217, ng/mL (IPMS) | NfL324 | 0.47 | <0.0001 | (0.27, 0.62) |
| CSF pTau217, ng/mL (IPMS) | NfL530 | 0.49 | <0.0001 | (0.31, 0.64) |
| NfL ELISA | NfL101 | 0.67 | <0.0001 | (0.53, 0.78) |
| NfL ELISA | NfL117 | 0.7 | <0.0001 | (0.56, 0.8) |
| NfL ELISA | NfL165 | 0.72 | <0.0001 | (0.6, 0.81) |
| NfL ELISA | NfL284 | 0.56 | <0.0001 | (0.38, 0.69) |
| NfL ELISA | NfL324 | 0.88 | <0.0001 | (0.81, 0.92) |
| NfL ELISA | NfL530 | 0.72 | <0.0001 | (0.59, 0.81) |
| NfL101 | NfL117 | 0.99 | <0.0001 | (0.99, 1) |
| NfL101 | NfL165 | 0.98 | <0.0001 | (0.97, 0.99) |
| NfL101 | NfL284 | 0.89 | <0.0001 | (0.84, 0.93) |
| NfL101 | NfL324 | 0.87 | <0.0001 | (0.8, 0.91) |
| NfL101 | NfL530 | 0.75 | <0.0001 | (0.64, 0.83) |
| NfL117 | NfL165 | 0.98 | <0.0001 | (0.97, 0.99) |
| NfL117 | NfL284 | 0.9 | <0.0001 | (0.84, 0.93) |
| NfL117 | NfL324 | 0.88 | <0.0001 | (0.82, 0.92) |
| NfL117 | NL530 | 0.76 | <0.0001 | (0.65, 0.84) |
| NfL165 | NfL284 | 0.86 | <0.0001 | (0.79, 0.91) |
| NfL165 | NfL324 | 0.88 | <0.0001 | (0.82, 0.92) |
| NfL165 | NfL530 | 0.81 | <0.0001 | (0.71, 0.87) |
| NfL284 | NfL324 | 0.71 | <0.0001 | (0.58, 0.8) |
| NfL284 | NfL530 | 0.67 | <0.0001 | (0.53, 0.77) |
| NfL324 | NfL530 | 0.75 | <0.0001 | (0.63, 0.83) |

The present example establishes there are at least 3 major NfL truncated species in CSF, and these are increased to varying degrees in AD. Further, brain NfL is full length, with a newly identified c-terminal fragment. The major CSF NfL species have different relationships with each other and other AD measures. This would indicate NfL truncated species could be differentially secreted in regard of NfL biology and neurodegeneration and some of them might be more relevant as biomarkers than others. NfL peptides level from NfL Coil 1 domain were correlated to each other and behaved slightly differently from peptides measured from Coil 2 and C-ter regions. A significant increase of NfL peptides 324 and 530 was found in symptomatic AD CSF supporting these domains might be more relevant as biomarkers. Higher correlation observed between NfL324 and NfL immunoassay, combined with the similar fold increase between AD and controls for NfL325 (1.5x) and the ELISA assay (1.4x) would suggest antibodies used by this Uman proprietary assay were likely selected to target these best performing NfL isoforms. There were modest correlations with CDR, age, and phosphorylated and total tau, while measures of amyloid PET and MMSE had low correlations.

### Example 8

In this example, Nfl isoforms from CSF samples obtained from control, ALS and SMA human subjects were analyzed. CSF samples from patients with sporadic amyotrophic lateral sclerosis (ALS, n=12), familial ALS (n=6), spinal muscular atrophy (SMA,n=10), and normal neurologic controls were analyzed for NfL species differences utilizing the same methods described. Briefly, 0.5 ng of 15N13C internal standard NfL was added to 0.5 ml CSF before immunoprecipitation with a 1:1:1 mixture of antibodies HJ30.13, HJ30.4 and HJ30.11. The processed samples were analyzed by LC-MS/MS to quantify the amount of NfL species at each region shown in FIGS. 57-63. These findings revealed increases in sporadic and familial ALS symptomatic cases more than asymptomatic cases, SMA, or controls. The amount of increase in NfL species was highly correlated with the rate of decline as measured by the ALS FRS scale as measured in decline per month. The ALS profile of NfL species showed increased amounts from amino acid position 37 through 352, and then another increase from 437 to 539 (the c-terminal peptide). However, the profile was different for controls with less pronounced c-terminal peptide amounts and other regions. Finally, the SMA profile showed very little mid-domain region, while the region from 339-461 was relatively increased. This indicates there may be specific profiles for each disease state that can be quantified and used for differential diagnosis.

1. A method for detecting neurofilament light chain (Nfl) in a biological sample, the method comprising
(a) providing a biological sample selected from a blood sample or a CSF sample;
(b) enriching for one to a plurality of Nfl isoforms in the biological sample; and
(c) detecting one to a plurality of Nfl isoforms enriched in step (b).

2. The method of claim 1,
wherein step (b) comprises
(i) contacting the biological sample with a first epitope-binding agent that specifically binds a first population of Nfl isoforms, and isolating the first population of Nfl isoforms thereby producing a sample depleted of the first population of Nfl isoforms;
(ii) contacting the sample depleted of the first population of Nfl isoforms with a second epitope-binding agent that specifically binds a second population of Nfl isoforms, and isolating the second population of Nfl isoforms thereby producing a sample depleted of the first and the second populations of Nfl isoforms; and
(iii) contacting the sample depleted of the first and second populations of Nfl isoforms with a third epitope-binding agent that specifically binds a third population of Nfl isoforms, and isolating the third population of Nfl isoforms;
wherein step (c) comprises detecting one to a plurality of Nfl isoforms in the first population of Nfl isoforms, one to a plurality of Nfl isoforms in the second population of Nfl isoforms, one to a plurality of Nfl isoforms in the third population of Nfl isoforms, or any combination thereof.

3. The method of claim 2, wherein
the first epitope-binding agent specifically binds to a first epitope within amino acids 90 to 300 of SEQ ID NO 1;
the second epitope-binding agent specifically binds to a second epitope within amino acids 200 to 450 of SEQ ID NO: 1 that is downstream of the first epitope; and
the third epitope-binding agent specifically binds to a third epitope within amino acids 397 to 543 of SEQ ID NO: 1 that is downstream of the second epitope.

4. (canceled)

5. (canceled)

6. The method of claim 1,
wherein step (b) comprises
(i) contacting the biological sample with a first epitope-binding agent that specifically binds a first population of Nfl isoforms, and isolating the first population of Nfl isoforms thereby producing a sample depleted of the first population of Nfl isoforms;
(ii) contacting the sample depleted of the first population of Nfl isoforms with a second epitope-binding agent that specifically binds a second population of Nfl isoforms, and isolating the second population of Nfl isoforms;
wherein step (c) comprises detecting one to a plurality of Nfl isoforms in the first population of Nfl isoforms, one to a plurality of Nfl isoforms in the second population of Nfl isoforms, or any combination thereof.

7. The method of claim 6, wherein
the first epitope-binding agent specifically binds to a first epitope within amino acids 90 to 450 of SEQ ID NO: 1;
the second epitope-binding agent specifically binds to an epitope within amino acids 396 to 543 of SEQ ID NO: 1.

8. (canceled)

9. (canceled)

10. (canceled)

11. (canceled)

12. The method of claim 1,
wherein step (b) comprises contacting the biological sample with an epitope-binding agent that specifically binds a first population of Nfl isoforms, and isolating the first population of Nfl isoforms; wherein the epitope-binding agent is selected from the group consisting of:
(i) an epitope-binding agent that specifically binds to an epitope within amino acids 90 to 250 of SEQ ID NO: 1 or amino acids 125 to 250 or SEQ ID NO: 1;
(ii) an epitope-binding agent that specifically binds to an epitope within amino acids 116 to 184 of SEQ ID NO: 1;
(iii) an epitope-binding agent that specifically binds to an epitope within amino acids 250 to 400 of SEQ ID NO: 1;
(iv) an epitope-binding agent that specifically binds to an epitope within amino acids 283 to 338 of SEQ ID NO: 1;
(v) an epitope-binding agent that specifically binds to an epitope within amino acids 400 to 543 of SEQ ID NO: 1;
(vi) an epitope-binding agent that specifically binds to an epitope within amino acids 437 to 543 of SEQ ID NO: 1;
(vii) HJ30.1 or an antigen-binding fragment thereof, HJ30.2 or an antigen-binding fragment thereof, HJ30.13 or an antigen-binding fragment thereof, or an epitope-binding agent that competitively inhibits HJ30.1, HJ30.2 or HJ30.13 binding to full-length, recombinant Nfl
(viii) HJ30.4 or an antigen-binding fragment thereof, HJ30.7 or an antigen-binding fragment thereof, or an epitope-binding agent that competitively inhibits HJ30.4 or HJ30.7 binding to full-length, recombinant Nfl; and
(ix) HJ30.11, an antigen-binding fragment thereof, or an epitope-binding agent that competitively inhibits HJ30.11 binding to full-length, recombinant Nfl.

13. (canceled)

14. (canceled)

15. (canceled)

16. (canceled)

17. (canceled)

18. (canceled)

19. (canceled)

20. The method of claim 1, wherein step (b) enriches for Nfl isoforms comprising an N-terminal truncation, wherein the N-terminal truncation for each Nfl isoform comprises an amino acid sequence independently selected from the group consisting of:
(a) amino acids 1 to 90 of SEQ ID NO: 1;
(b) amino acids 1 to 92 of SEQ ID NO: 1;
(c) amino acids 1 to 124 of SEQ ID NO: 1;
(d) amino acids 1 to 135 of SEQ ID NO: 1;
(e) amino acids 1 to 137 of SEQ ID NO: 1;
(f) amino acids 1 to 146 of SEQ ID NO: 1;
(g) amino acids 1 to 162 of SEQ ID NO: 1;
(h) amino acids 1 to 222 of SEQ ID NO: 1;
(i) amino acids 1 to 223 of SEQ ID NO: 1;
(j) amino acids 1 to 234 of SEQ ID NO: 1;
(k) amino acids 1 to 252 of SEQ ID NO: 1;
(l) amino acids 1 to 271 of SEQ ID NO: 1;
(m) amino acids 1 to 281 of SEQ ID NO: 1;
(n) amino acids 1 to 323 of SEQ ID NO: 1;
(o) amino acids 1 to 339 of SEQ ID NO: 1;
(p) amino acids 1 to 359 of SEQ ID NO: 1;
(q) amino acids 1 to 396 of SEQ ID NO: 1;
(r) amino acids 1 to 399 of SEQ ID NO: 1;
(s) amino acids 1 to 420 of SEQ ID NO: 1;
(t) amino acids 1 to 436 of SEQ ID NO: 1; or
(u) amino acids 1 to 528 of SEQ ID NO: 1.

21. (canceled)

22. (canceled)

23. (canceled)

24. The method of claim 1, wherein the method further comprises in step (c):
cleaving the enriched Nfl isoforms with a protease and then optionally desalting the resultant cleavage product by solid phase extraction to obtain a sample comprising proteolytic peptides of Nfl; and
performing liquid chromatography-mass spectrometry (LC/MS) of the sample comprising proteolytic peptides of Nfl to detect and optionally quantify the amount of at least one proteolytic peptide of Nfl.

25. (canceled)

26. (canceled)

27. (canceled)

28. (canceled)

29. (canceled)

30. (canceled)

31. A method of detecting a biomarker in a sample obtained from a subject, the method comprising detecting neurofilament light chain according to the method of claim 1, wherein the biological sample is a sample obtained from a subject having or at risk of having neuronal damage and the biomarker is the one to the plurality of Nfl isoforms detected in step (c).

32. (canceled)

33. (canceled)

34. (canceled)

35. The method of claim 31 further comprising determining if the amount of the quantified biomarker is reduced in comparison to its level in control subjects who are cognitively normal, in control subjects who are cognitively normal and amyloid negative, or in control subjects who are cognitively normal and negative for pathological levels of tau deposits in the brain as evaluated by PET.

36. (canceled)

37. The method of claim 31, the method further comprising treating the subject.

38. A method of measuring a treatment response in a subject having or at risk of having neuronal damage, the method comprising
(a) quantifying, in a first biological sample obtained from the subject, a biomarker according to claim 31;
(b) administering a treatment to the subject; and
(c) quantifying, in a second biological sample obtained from the subject after the treatment, the biomarker quantified in step (a);
wherein the first biological sample and the second biological sample are both a blood sample or both a CSF sample; and
wherein no change or a decrease in the amount of the biomarker in the second sample, as compared to the first sample, indicates a positive treatment response, or wherein the amount of the biomarker increases in the second sample as compared to the first sample but the change is less than a change that occurs in a control group of subjects that have neuronal injury but were not administered treatment.

39. A method of monitoring a subject having or at risk of having neuronal damage, the method comprising quantifying a biomarker according to claim 31, in a first biological sample obtained from the subject and a second biological sample obtained from the subject, wherein the first biological sample and the second biological sample are both a blood sample or both a CSF sample, and wherein the second biological sample was obtained after the first biological sample;
wherein an increase in the amount of the biomarker in the second sample as compared to the first sample indicates an increase in neuronal damage.

40. (canceled)

41. (canceled)

42. An isolated anti-neurofilament light chain (Nfl) epitope-binding agent selected from
(a) HJ30.1, a humanized version of HJ30.1, or an epitope-binding agent that competitively inhibits HJ30.1 binding to full-length recombinant Nfl;
(b) HJ30.2, a humanized version of HJ30.2, or an epitope-binding agent that competitively inhibits HJ30.2 binding to full-length recombinant Nfl;
(c) HJ30.4, a humanized version of HJ30.4, or an epitope-binding agent that competitively inhibits HJ30.4 binding to full-length recombinant Nfl;
(d) HJ30.7, a humanized version of HJ30.7, or an epitope-binding agent that competitively inhibits HJ30.7 binding to full-length recombinant Nfl
(e) HJ30.11, a humanized version of HJ30.11, or an epitope-binding agent that competitively inhibits HJ30.11 binding to full-length recombinant Nfl; or
(f) HJ30.13, a humanized version of HJ30.13, or an epitope-binding agent that competitively inhibits HJ30.13 binding to full-length recombinant Nfl.

43. (canceled)

44. (canceled)

45. (canceled)

46. (canceled)

47. (canceled)

48. An isolated antibody produced by the hybridoma deposited with the American Type Culture Collection having
(a) the ATCC Designation PTA-126966;
(b) the ATCC Designation PTA-126967;
(c) the ATCC Designation PTA-126968;
(d) the ATCC Designation PTA-126969;
(e) the ATCC Designation PTA-126970; or
(f) the ATCC Designation PTA-126971.

49. (canceled)

50. (canceled)

51. (canceled)

52. (canceled)

53. (canceled)

54. An isolated anti-neurofilament light chain (Nfl) comprising
(a) a light chain variable region of HJ30.1 (ATCC #PTA-126966), and a heavy chain variable region of HJ30.1 (ATCC #PTA-126966);
(b) a light chain variable region of HJ30.2 (ATCC #PTA-126967), and a heavy chain variable region of HJ30.2 (ATCC #PTA-126967);
(c) a light chain variable region of HJ30.4 (ATCC #PTA-126968), and a heavy chain variable region of HJ30.4 (ATCC #PTA-126968);
(d) a light chain variable region of HJ30.7 (ATCC #PTA-126969), and a heavy chain variable region of HJ30.7 (ATCC #PTA-126969);
(e) a light chain variable region of HJ30.11 (ATCC #PTA-126970), and a heavy chain variable region of HJ30.11 (ATCC #PTA-126970); or
(f) a light chain variable region of HJ30.13 (ATCC #PTA-126971), and a heavy chain variable region of HJ30.13 (ATCC #PTA-126971).

55. (canceled)

56. (canceled)

57. (canceled)

58. (canceled)

59. (canceled)

60. An isolated anti-neurofilament light chain (Nfl) comprising
(a) a light chain variable region with 90, 91, 92, 93, 94, 95, 96, 97, 98, 99, or 100% identity to the VL of HJ30.1 (ATCC #PTA-126966), and a heavy chain variable region with 90, 91, 92, 93, 94, 95, 96, 97, 98, 99 or 100% identity to the VH of HJ30.1 (ATCC #PTA-126966);
(b) a light chain variable region with 90, 91, 92, 93, 94, 95, 96, 97, 98, 99, or 100% identity to the VL of HJ30.2 (ATCC #PTA-126967), and a heavy chain variable region with 90, 91, 92, 93, 94, 95, 96, 97, 98, 99 or 100% identity to the VH of HJ30.2 (ATCC #PTA-126967);
(c) a light chain variable region with 90, 91, 92, 93, 94, 95, 96, 97, 98, 99, or 100% identity to the VL of HJ30.4 (ATCC #PTA-126968), and a heavy chain variable region with 90, 91, 92, 93, 94, 95, 96, 97, 98, 99 or 100% identity to the VH of HJ30.4 (ATCC #PTA-126968);
(d) a light chain variable region with 90, 91, 92, 93, 94, 95, 96, 97, 98, 99, or 100% identity to the VL of HJ30.7 (ATCC #PTA-126969), and a heavy chain variable region with 90, 91, 92, 93, 94, 95, 96, 97, 98, 99 or 100% identity to the VH of HJ30.7 (ATCC #PTA-126969);
(e) a light chain variable region with 90, 91, 92, 93, 94, 95, 96, 97, 98, 99, or 100% identity to the VL of HJ30.11 (ATCC #PTA-126970), and a heavy chain variable region with 90, 91, 92, 93, 94, 95, 96, 97, 98, 99 or 100% identity to the VH of HJ30.11 (ATCC #PTA-126970); or
f) a light chain variable region with 90, 91, 92, 93, 94, 95, 96, 97, 98, 99, or 100% identity to the VL of HJ30.13 (ATCC #PTA-126971), and a heavy chain variable region with 90, 91, 92, 93, 94, 95, 96, 97, 98, 99 or 100% identity to the VH of HJ30.13 (ATCC #PTA-126971).

61. (canceled)

62. (canceled)

63. (canceled)

64. (canceled)

65. (canceled)

66. A method of detecting and/or quantifying Nfl in a biological sample, the method comprising contacting the biological sample with the Use of an epitope-binding agent of claim 42.

67. (canceled)

68. (canceled)

69. A kit, comprising
(a) one or more antibodies selected from
(i) HJ30.1, a humanized version of HJ30.1, or an epitope-binding agent that competitively inhibits HJ30.1 binding to full-length recombinant Nfl;
(ii) HJ30.2, a humanized version of HJ30.2, or an epitope-binding agent that competitively inhibits HJ30.2 binding to full-length recombinant Nfl;
(iii) HJ30.4, a humanized version of HJ30.4, or an epitope-binding agent that competitively inhibits HJ30.4 binding to full-length recombinant Nfl;
(iv) HJ30.7, a humanized version of HJ30.7, or an epitope-binding agent that competitively inhibits HJ30.7 binding to full-length recombinant Nfl
(v) HJ30.11, a humanized version of HJ30.11, or an epitope-binding agent that competitively inhibits HJ30.11 binding to full-length recombinant Nfl; or
(vi) HJ30.13, a humanized version of HJ30.13, or an epitope-binding agent that competitively inhibits HJ30.13 binding to full-length recombinant Nfl; and
(b) instructions for detecting neurofilament light chain (Nfl) in a biological sample according to claim 1.

70. A kit, comprising
(a) one or more antibodies selected from
(i) HJ30.1, a humanized version of HJ30.1, or an epitope-binding agent that competitively inhibits HJ30.1 binding to full-length recombinant Nfl;
(ii) HJ30.2, a humanized version of HJ30.2, or an epitope-binding agent that competitively inhibits HJ30.2 binding to full-length recombinant Nfl;
(iii) HJ30.4, a humanized version of HJ30.4, or an epitope-binding agent that competitively inhibits HJ30.4 binding to full-length recombinant Nfl;
(iv) HJ30.7, a humanized version of HJ30.7, or an epitope-binding agent that competitively inhibits HJ30.7 binding to full-length recombinant Nfl
(v) HJ30.11, a humanized version of HJ30.11, or an epitope-binding agent that competitively inhibits HJ30.11 binding to full-length recombinant Nfl; or
(vi) HJ30.13, a humanized version of HJ30.13, or an epitope-binding agent that competitively inhibits HJ30.13 binding to full-length recombinant Nfl; and
(b) instructions for measuring a treatment response in a subject having or at risk of having neuronal damage according to claim 38.
